(19) United States
(12) Patent Application Publication
(10) Pub. No.: US 2024/0200019 A1
(43) Pub. Date: Jun. 20, 2024

(54) GENETICALLY MODIFIED YEAST CELLS AND METHODS OF USE FOR INCREASED LIPID YIELD

(71) Applicant: Ginkgo Bioworks, Inc., Boston, MA (US)

(72) Inventors: Annapurna Kamineni, Arlington, MA (US); Arthur J. Shaw, Belmont, MA (US); Vasiliki Tsakraklides, Lexington, MA (US)

(21) Appl. No.: 18/288,954

(22) PCT Filed: Apr. 28, 2022
(86) PCT No.: PCT/US22/26807

§ 371 Date: Oct. 30, 2023

Related U.S. Application Data
(60) Provisional application No. 63/181,895, filed on Apr. 29, 2021.

(51) Int. Cl. C12N 1/16; C12N 9/10; C12N 9/88; C12N 15/81; C12P 5/00; C12P 7/6454; C12P 7/6463; C12R 1/645

(52) U.S. Cl. CPC C12N 1/165; C12N 9/1029; C12N 9/88; C12N 15/815; C12P 5/007; C12P 7/6454; C12P 7/6463; C07K 2319/00; C12R 2001/645; C12Y 203/01008; C12Y 401/02009

(57) ABSTRACT

Aspects of the disclosure are directed to genetically modified yeast cells and methods for use. Certain aspects are directed to recombinant yeast cells comprising exogenous nucleic acid sequences encoding phosphotransacetylase and/or phosphoketolase proteins, including a phosphoketolase protein from Clostridium acetobutylicum. Also disclosed are methods for generating recombinant yeast cells and methods of use of such cells for production of one or more products, including lipids, oils, fatty acids, and triacylglycerides.

This application claims priority to U.S. Provisional Patent Application Ser. No. 63/181,895, filed Apr. 29, 2021, which is incorporated by reference herein in its entirety.

### SEQUENCE LISTING

The instant application contains a Sequence Listing which has been submitted in ASCII format and is hereby incorporated by reference in its entirety. Said ASCII copy, created on Apr. 25, 2022, is named GIBWP0008WO_ST25.txt and is 27,414 bytes in size.

### BACKGROUND

### I. Field of the Invention

Aspects of this invention relate to at least the fields of microbiology, genetics, biotechnology, and biochemistry.

### II. Background

Lipids are important precursors in food, cosmetics, biodiesel and biochemical industries [1]. The ever-increasing demands of these industries are largely fulfilled by plant oil feedstocks [1,2]. Plantations for oil are dependent on climatic changes, geopolitics and require large arable lands. Oil plantations are continually displacing large areas of tropical forests worldwide, severely affecting their regional biodiversity [2,3]. These environmental and sustainability concerns have fueled research into microbial oil as an alternative source of lipid production [4,5].

De novo fatty acid synthesis is illustrated in FIGS. 1 and FIGS. 2A-2B. To make one molecule of triolein (C₅₇H₁₀₄O₆) using this biochemical pathway, 27 acetyl-CoA and at least 48 NADPH molecules are needed (FIG. 2A). To produce the required intermediates, the native lipid pathway utilizes 18 glucose molecules (FIGS. 2A and 2B).

Another strategy to improve glucose-to-lipid production involves phosphoketolase (Xpk, EC 4.1.2.9, EC 4.1.2.22), which converts fructose 6-phosphate (F6P) and/or the PPP intermediate xylulose 5-phosphate (X5P) to acetyl phosphate (AcP) and glyceraldehyde 3-phosphate (Ga3P), and phosphotransacetylase (Pta, EC 2.3.1.8), which catalyzes the reversible conversion of AcP to acetyl-CoA. The combined activities of Xpk and Pta produce cytosolic acetyl-CoA from the PPP instead of glycolysis. Overall, the Xpk/Pta pathway requires 2.3 fewer moles of glucose to make one mole of triolein compared to the native pathway.

In implementations of the Xpk/Pta pathway in E. coli, 11 gene overexpressions, 9 gene deletions, and over 50 genomic mutations accumulated through evolution were engineered to successfully rescue growth in a glycolytic mutant through the Xpk/Pta pathway [31]. Thus, lipid production by microbial cells can be a challenge.

There exists a need for microbial cells such as yeast cells with improved lipid production.

### SUMMARY

The present disclosure fulfils certain needs by providing, inter alia, recombinant yeast cells having improved lipid production, including recombinant Yarrowia lipolytica cells, which express functional, biologically active phosphoketolase and phosphotransacetylase proteins. Aspects of the disclosure are directed to recombinant yeast cells expressing a phosphotransacetylase protein (e.g., from Thermoanaerobacterium saccharolyticum or Bacillus subtilis) and a phosphoketolase protein (e.g., from Clostridium acetobutylicum). Also disclosed are codon optimized sequences encoding for a phosphoketolase protein from Clostridium acetobutylicum having surprising activity in Yarrowia lipolytica. The disclosed cells in some cases do not express, or have reduced expression of, an endogenous functional phosphofructokinase protein. Methods for making and using such recombinant yeast cells, as well as nucleic acids, vectors, and reagents for use in such methods, are also disclosed.

Embodiments of the disclosure include recombinant cells, nucleic acids, vectors, methods for expressing a nucleic acid sequence, methods for generating a recombinant cell, methods for culturing a recombinant cell, methods for expressing an exogenous gene, methods for deleting an endogenous gene, methods for reducing expression of an endogenous gene, methods for modifying expression of an endogenous gene, methods for collecting a product from a recombinant cell, methods for increasing lipid production, methods for modifying the lipid composition of a cell, and methods for using a recombinant cell. Embodiments include nucleic acid molecules comprising one or more sequences (e.g., promoter sequences, coding sequences, etc.). Embodiments include nucleic acid molecules comprising nucleic acid sequences encoding a phosphoketolase protein (e.g., a phosphoketolase protein from Clostridium acetobutylicum) and/or a phosphotransacetylase protein. Embodiments also include recombinant, transformed, or modified cells, vectors, and/or expression cassettes comprising such nucleic acid molecules.

Nucleic acids of the present disclosure can include at least 1, 2, 3, 4, or more of the following components: a promoter, a terminator, a coding sequence, an antibiotic resistance gene, a nucleic acid sequence encoding for a phosphoketolase protein, and a nucleic acid sequence encoding for a phosphotransacetylase protein. One or more of these components may be excluded from nucleic acids of the disclosure.

Recombinant cells of the present disclosure can include at least one or more of the following components: an exogenous nucleic acid sequence encoding a phosphoketolase protein, an exogenous nucleic acid sequence encoding a phosphotransacetylase protein, a phosphoketolase protein, a phosphotransacetylase protein, a nucleic acid encoding a fusion protein, a fusion protein, and a deletion in a phosphofructokinase gene. Recombinant cells of the disclosure may include 1, 2, 3, 4, 5, 6, 7, 8, 9, 10, or more copies of a sequence encoding a phosphoketolase protein. Recombinant cells of the disclosure may include 1, 2, 3, 4, 5, 6, 7, 8, 9, 10, or more copies of a sequence encoding a phosphotransacetylase protein. Recombinant cells of the disclosure may express a phosphoketolase protein and a phosphotransacetylase protein as separate proteins. Recombinant cells of the disclosure may express a phosphoketolase protein and a phosphotransacetylase protein as a fusion protein, which may comprise the phosphoketolase protein and the phosphotransacetylase protein attached via a linker. Any one or more of the preceding components may be excluded from recombinant cells in particular embodiments.

Methods of the present disclosure can include at least 1, 2, 3, 4, or more of the following steps: transforming a cell, culturing a cell, eliminating expression of a functional native protein in a cell, expressing an exogenous nucleic acid sequence in a cell, measuring lipid production in a cell, and collecting a product from a cell. Any one or more of the preceding steps may be excluded from the disclosed methods.

Disclosed herein, in some embodiments, is a recombinant yeast cell comprising (a) a first exogenous nucleic acid sequence encoding a phosphoketolase protein comprising a sequence having at least 90% sequence identity with SEQ ID NO:28; and (b) a second exogenous nucleic acid sequence encoding a phosphotransacetylase protein. Also disclosed is a method for generating the recombinant yeast cell comprising transforming a yeast cell with the first exogenous nucleic acid sequence and the second exogenous nucleic acid sequence. In some embodiments, the method further comprises culturing the recombinant yeast cell under conditions sufficient to express the first exogenous nucleic acid sequence and the second exogenous nucleic acid sequence. In some embodiments, the method comprises culturing the recombinant yeast cell for at least or at most 2, 3, 4, 5, 6, 7, 8, 9, 10, 11, 12, 13, 14, 15, 16, 17, 18, 19, or 20 passages, or any range derivable therein. In some embodiments, the method comprises culturing the recombinant yeast cell for at least 10 passages. Also disclosed herein, in some embodiments, is a method for collecting an acetyl-CoA derived product from a yeast cell, the method comprising (a) culturing a recombinant yeast cell comprising (i) a first exogenous nucleic acid sequence encoding a phosphoketolase protein comprising a sequence having at least 90% sequence identity with SEQ ID NO:28; and (ii) a second exogenous nucleic acid sequence encoding a phosphotransacetylase protein under conditions sufficient to express the first exogenous nucleic acid sequence and the second exogenous nucleic acid sequence; and (b) collecting the product from the yeast cell, wherein the product is an oil, a lipid, a fatty acid, a fatty alcohol, or a triacylglyceride. In some embodiments, the yeast cell is cultured for at least or at most 2, 3, 4, 5, 6, 7, 8, 9, 10, 11, 12, 13, 14, 15, 16, 17, 18, 19, or 20 passages, or any range derivable therein. In some embodiments, the yeast cell is cultured for at least 10 passages. In some embodiments, the acetyl-CoA derived product is an oil. In some embodiments, the acetyl-CoA derived product is a lipid. In some embodiments, the acetyl-CoA derived product is a fatty acid. In some embodiments, the acetyl-CoA derived product is a fatty alcohol. In some embodiments, the acetyl-CoA derived product is a triacylglyceride. In some embodiments, the acetyl-CoA derived product is triolein.

In some embodiments, the phosphoketolase protein comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or more sequence identity with SEQ ID NO:28, or any range or value derivable therein. In some embodiments, the phosphoketolase protein comprises a sequence having at least 95% sequence identity with SEQ ID NO:28. In some embodiments, the phosphoketolase protein comprises a sequence having at least 98% sequence identity with SEQ ID NO:28. In some embodiments, the phosphoketolase protein comprises a sequence having at least 99% sequence identity with SEQ ID NO:28. In some embodiments, the phosphoketolase protein comprises SEQ ID NO:28. In some embodiments, the phosphoketolase protein is a phosphoketolase protein from Clostridium acetobutylicum.

In some embodiments, the first exogenous nucleic acid sequence and the second exogenous nucleic acid sequence are on a single expression cassette. In some embodiments, the first exogenous nucleic acid sequence and the second exogenous nucleic acid sequence are on different expression cassettes. In some embodiments, the first exogenous nucleic acid sequence comprises a sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or more sequence identity with SEQ ID NO:26, or any range or value derivable therein. In some embodiments, the first exogenous nucleic acid sequence comprises SEQ ID NO:26. In some embodiments, the first exogenous nucleic acid sequence comprises two copies of SEQ ID NO:26. In some embodiments, the first exogenous nucleic acid sequence comprises three copies of SEQ ID NO:26. the first exogenous nucleic acid sequence comprises 1, 2, 3, 4, 5, 6, 7, 8, 9, 10, or more copies of SEQ ID NO:26. In some embodiments, the first exogenous nucleic acid sequence comprises a sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or more sequence identity with SEQ ID NO:27 or any range or value derivable therein. In some embodiments, the first exogenous nucleic acid sequence comprises SEQ ID NO:27. In some embodiments, the first exogenous nucleic acid sequence comprises two copies of SEQ ID NO:27. In some embodiments, the first exogenous nucleic acid sequence comprises three copies of SEQ ID NO:27. In some embodiments, the first exogenous nucleic acid sequence comprises 1, 2, 3, 4, 5, 6, 7, 8, 9, 10, or more copies of SEQ ID NO:27.

In some embodiments, the phosphotransacetylase protein comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or more sequence identity with SEQ ID NO:29. In some embodiments, the first exogenous nucleic acid sequence comprises a sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or more sequence identity with SEQ ID NO:23 or any range or value derivable therein. In some embodiments, the second exogenous nucleic acid sequence comprises SEQ ID NO:23. In some embodiments, the second exogenous nucleic acid sequence comprises two copies of SEQ ID NO:23. In some embodiments, the phosphotransacetylase protein is a phosphotransacetylase protein from Bacillus subtilis.

In some embodiments, the phosphotransacetylase protein comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or more sequence identity with SEQ ID NO:29. In some embodiments, the first exogenous nucleic acid sequence comprises a sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity with SEQ ID NO:24 or any range or value derivable therein. In some embodiments, the second exogenous nucleic acid sequence comprises SEQ ID NO:24. In some embodiments, the second exogenous nucleic acid sequence comprises two copies of SEQ ID NO:24. In some embodiments, the first exogenous nucleic acid sequence comprises a sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity with SEQ ID NO:25 or any range or value derivable therein. In some embodiments, the second exogenous nucleic acid sequence comprises SEQ ID NO:25. In some embodiments, the second exogenous nucleic acid sequence comprises two copies of SEQ ID NO:25. In some embodiments, the second exogenous nucleic acid sequence comprises three copies of SEQ ID NO:25. In some embodiments, the second exogenous nucleic acid sequence comprises 1, 2, 3, 4, 5, 6, 7, 8, 9, 10, or more copies of SEQ ID NO:25. In some embodiments, the phosphotransacetylase protein is a phosphotransacetylase protein from Thermoanaerobacterium saccharolyticum.

In some embodiments, the recombinant yeast cell has a reduced expression of an endogenous functional phosphofructokinase protein compared to a wild-type yeast cell. In some embodiments, the recombinant yeast cell has at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% reduced expression of an endogenous functional phosphofructokinase protein compared to a wild-type yeast cell, or any range or value derivable therein. In some embodiments, the recombinant yeast cell does not express an endogenous functional phosphofructokinase protein. In some embodiments, the recombinant yeast cell expresses a native functional aldehyde dehydrogenase protein. In some embodiments, the recombinant yeast cell expresses a native functional glycerol-3-phosphate phosphatase protein. In some embodiments, the recombinant yeast cell is Arxula adeninivorans, Saccharomyces cerevisiae, or Yarrowia lipolytica. In some embodiments, the recombinant yeast cell is Yarrowia lipolytica.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention. As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that embodiments described herein in the context of the term “comprising” may also be implemented in the context of the term “consisting of” or “consisting essentially of.”

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary, Detailed Description of the Embodiments, Claims, and Brief Description of the Drawings.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows a schematic of the lipid synthesis pathway for triolein production in Y. lipolytica. The native glucose to lipid synthesis pathway is shown in solid lines, the pentose phosphate pathway is shown in dotted lines, the heterologous Xpk/Pta pathway is shown in dashed lines, and the PFK1 deletion is shown with an “X”. List of enzymes: ACL—ATP:citrate lyase, CIT—Citrate Synthase, ELO—Elongase, FAS—Fatty acid synthase, FBP—Fructose 1,6-bisphosphatase, OLE—Δ9 fatty acid desaturase, PDH—Pyruvate dehydrogenase, PFK—Phosphofructokinase, PGI—Phosphoglucoisomerase, PTA—Phosphotransacetylase, XPK—Phosphoketolase. Abbreviations: DHAP—Dihydroxyacetone phosphate, Ga3P—Glyceraldehyde 3-phosphate, Ma-CoA—Malonyl-CoA.

FIGS. 2A-2C show schematics of various biochemical pathways in Y. lipolytica. FIG. 2A shows a schematic of the pathway of triolein synthesis from acetyl-CoA in Y. lipolytica. A total of 27 Acetyl-CoA, 48 NADPH and 1 Glycerol-3-P are used to make 1 Triolein. FIG. 2B shows a schematic of acetyl-CoA production in Y. lipolytica using the native pathway. Glycolysis, partial TCA cycle reactions and ATP:Citrate Lyase (ACL) are involved in the production of acetyl-CoA. NADPH requirement is fulfilled by the pentose phosphate pathway. FIG. 2C shows acetyl-CoA production in Y. lipolytica using the Xpk/Pta/Δpfk1 pathway. The Xpk/Pta pathway ties acetyl-CoA production to NADPH production, reducing the dependency on pentose phosphate pathway for NADPH production. PFK1 deletion partially disables glycolysis forcing carbon flux through the heterologous pathway. Glyceraldehyde 3-phosphate (Ga3P) made by the Xpk/Pta pathway can still be metabolized via glycolysis to produce acetyl-CoA and glycerol 3-phosphate. This route utilizes 15.667 molecules of glucose to make 1 molecule of triolein resulting in a lipid yield of 0.31 g/g. Ga3P can also be recycled through the Xpk/Pta pathway via the fructose 1,6-bisphosphatase step but a complete recycle of all Ga3P would result in a redox imbalance (not shown). The Ga3P flux could potentially be split between the two routes (recycle through Xpk/Pta and glycolysis). An appropriate flux distribution could have a balanced redox and a positive impact on lipid yield.

FIG. 3 shows heterologous Pta activity in Y. lipolytica. PTA genes from seven different source organisms codon optimized to S. cerevisiae (GeneArt) were expressed in YB-392 under the control of the Y. lipolytica EXPI promoter using linear integrating expression cassettes. Cell-free extracts (CFE) from four transformants per test gene were analyzed for Pta activity using a DTNB assay. Data is presented as fold change of the measured specific activity over the averaged specific activity of the parent strain YB-392.

FIG. 4 shows heterologous Xpk activity in Y. lipolytica. XPK genes from four source organisms codon optimized to Y. lipolytica (ATGme [57]) were expressed in YB-392 under the control of the Y. lipolytica TEF1 promoter on replicating plasmids. Cell-free extracts from 4 transformants per test gene were analyzed by ferric hydroxamate assay to measure Xpk activity on ribose 5-phosphate (R5P, black bars) and fructose 6-phosphate (F6P, grey bars). Absorbance was measured at 540 nm and normalized to total protein in the crude cell-free extract after a reaction time of 30 min. Data is presented as fold change of the normalized absorbance over the averaged normalized absorbance of the parent strain YB-392 (dashed line).

FIGS. 5A-5C show Y. lipolytica growth on glucose. Strains YB-392 (wild-type) and NS1047 (Δpfk1) were streaked on Yeast Nitrogen Base (YNB) media containing 2% glucose for 4 days (FIG. 5A), or YPD for 2 days (FIG. 5B) as labeled in FIG. 5C.

FIGS. 6A and 6B shows results from engineering the Xpk/Pta pathway in a Δpfk1 Y. lipolytica strain. FIG. 6A shows Pta and Xpk activity. Pta activity in all strains shown was measured using the DTNB assay (black bars). Xpk activity in the control strain YB-392 and all the strains that obtained a copy of CaXPK through transformation (NS1281, NS1292, NS1322, NS1457 and NS1475) was measured using the ferric hydroxamate assay with ribose 5-phosphate as the substrate (grey bars). NS1047, NS1341, NS1352 and NS1420 were excluded from this assay. FIG. 6B shows growth and lipid accumulation assays. OD₆₀₀ was measured after 2 days of growth in lipid production media (black bars). Lipid accumulation was measured as fluorescence/OD after overnight growth in glycerol followed by seven days of culture in modified Verduyn media (grey bars). Modified Verduyn media contained glucose as the only carbon source and no nitrogen to induce lipid production.

FIGS. 7A-7B. FIG. 7A shows distribution plot of rare codons (≤1%) among two groups of genes, the highest expressers and moderate-low expressers. To understand codon usage among the highest expressed genes in Y. lipolytica, the published dataset generated by Ochoa-Estopier et al. (J Biotechnol. 2014 Jan. 20; 170:35-41) was analyzed. In that experiment, Y. lipolytica W29 was cultivated under chemostat and decelerostat (Dstat) conditions. The sum of the transcript levels measured across all the timepoints during the experiment was used to rank ˜6500 genes. Two groups of genes were compared, 20 of the highest expressed genes (ranks 1-20) with 20 genes of moderate-low expression (ranked ˜3000). The number of codons present were recorded at a frequency≤ 1% (rare codons) for each of these genes using ATGme. The distribution plot of the rare codons across these two groups of genes is shown. FIG. 7B shows results from testing of differently codon optimized versions of CaXPK in Y. lipolytica. Genes were either integrated into the chromosome or expressed on a replicating plasmid.

FIGS. 8A-8D shows characterization of Xpk/Pta/Δpfk1 strain NS1475 and YB-392 in 1-L glucose batch fermentation. FIG. 8A shows time-course profiles of glucose consumption, lipid free dry cell weight (LFDCW) and lipid titers of YB-392 and NS1475 over a five-day fermentation. FIGS. 8B-8D show lipid content (FIG. 8B), total lipid yield (FIG. 8C), and cell-specific lipid productivity (day 2-day 5) (FIG. 8D). Data are mean±standard deviation for two replicate runs.

FIGS. 9A-9E show results from the rebuilding of Xpk/Pta/Δpfk1 in Y. lipolytica with TsPTA(v2) and CaXPK(v2), as described in Example 4. FIG. 9A shows a strain construction flowchart. FIG. 9B shows time-course profiles of glucose consumption, lipid free dry cell weight (LFDCW), and lipid titers of Xpk/Pta/Δpfk1 strains NS1656 and NS1657 and strain YB-392 in 1-L glucose batch fermentations over a five-day period. FIG. 9C shows total lipid yield. FIG. 9D shows cell-specific lipid productivity (day 2-day 5). FIG. 9E shows lipid content. Data are mean±standard deviation for two replicate runs.

FIGS. 10A-10C show heterologous Xpk activity in Y. lipolytica. FIG. 10A shows activity of Xpk from various source organisms. XPK genes from the shown source organisms codon optimized to S. cerevisiae (GeneArt) were expressed in YB-392 under the control of the Arxula adeninivorans TEF1 promoter using linear integrating cassettes. Cell-free extracts from 4 transformants per test gene were analyzed by ferric hydroxamate assay to measure Xpk activity on ribose 5-phosphate (R5P, black bars). Absorbance was measured at 540 nm and normalized to total protein in the crude cell-free extract after a reaction time of 30 min. Data is presented as fold change of the normalized absorbance over the averaged normalized absorbance of the parent strain YB-392. FIG. 10B shows activity of Xpk from various source organisms. XPK genes from the shown source organisms codon optimized to S. cerevisiae (GeneArt) were expressed in YB-392 under the control of the Y. lipolytica TEF1 promoter using linear integrating cassettes. Cell-free extracts from 4 transformants per test gene were analyzed by ferric hydroxamate assay to measure Xpk activity on ribose 5-phosphate (R5P, black bars). Absorbance was measured at 540 nm and normalized to total protein in the crude cell-free extract after a reaction time of 30 min. Data is presented as fold change of the normalized absorbance over the averaged normalized absorbance of the parent strain YB-392. FIG. 10C shows activity of Xpk from various source organisms. XPK genes from the shown source organisms were codon optimized to S. cerevisiae (GeneArt) were expressed in YB-392 under the control of the Y. lipolytica TEF1 promoter on replicating plasmids. Cell-free extracts from 3-4 transformants per test gene were analyzed by ferric hydroxamate assay to measure Xpk activity on ribose 5-phosphate (R5P, black bars) and fructose 6-phosphate (F6P, grey bars). Absorbance was measured at 540 nm and normalized to total protein in the crude cell-free extract after a reaction time of 30 min. Data is presented as fold change of the normalized absorbance over the averaged normalized absorbance of the parent strain YB-392.

FIG. 11 shows heterologous Xpk activity in A. adeninivorans. XPK genes from the shown source organisms codon optimized to S. cerevisiae (GeneArt) were expressed in A. adeninivorans under the control of the Arxula adeninivorans TEF1 promoter using linear integrating cassettes. Cell-free extracts from 4 transformants per test gene were analyzed by ferric hydroxamate assay to measure Xpk activity on ribose 5-phosphate (R5P, black bars). Absorbance was measured at 540 nm and normalized to total protein in the crude cell-free extract after a reaction time of 30 min. Data is presented as fold change of the normalized absorbance over the averaged normalized absorbance of the parent strain.

### DETAILED DESCRIPTION OF THE INVENTION

The present disclosure is based, at least in part, on effective engineering of the Xpk/Pta pathway in a glycolysis-deficient Δpfk Y. lipolytica strain using exogenous nucleic acid sequences encoding phosphoketolase (Xpk; e.g., Xpk from Clostridium acetobutylicum) and phosphotransacetylase (Pta, e.g., Pta from Bacillus subtilis and/or Thermoanaerobacterium saccharolyticum) proteins. Aspects of the disclosure are directed to recombinant yeast cells expressing functional Xpk and Pta. Such cells may have increased lipid yields compared with wild-type yeast cells. In some cases, the disclosed recombinant yeast cells also have reduced expression of an endogenous phosphofructokinase (pfk) protein. Methods for producing and collecting lipid products from such cells are also disclosed.

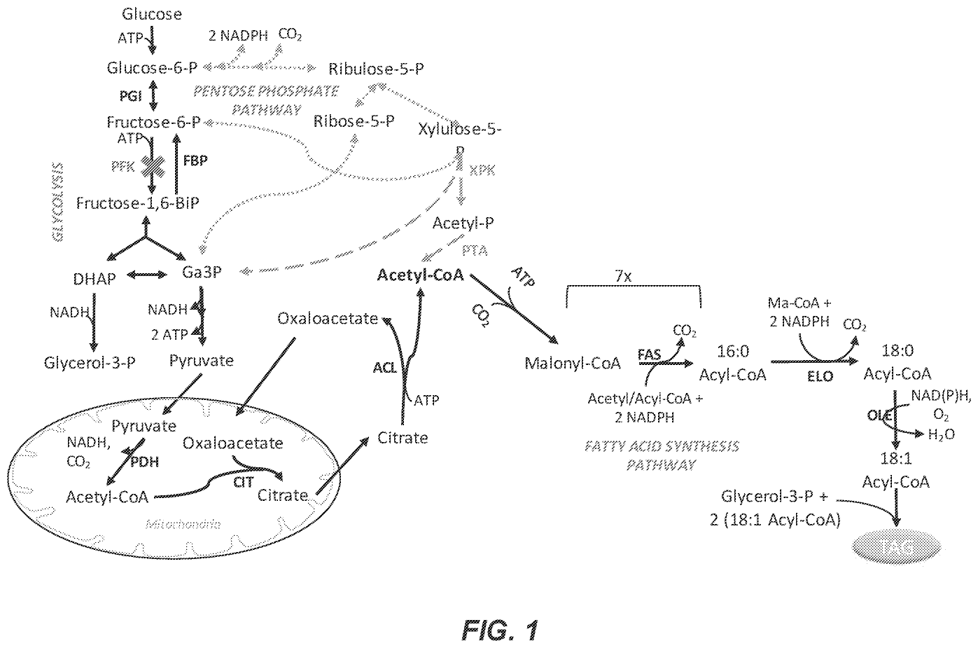

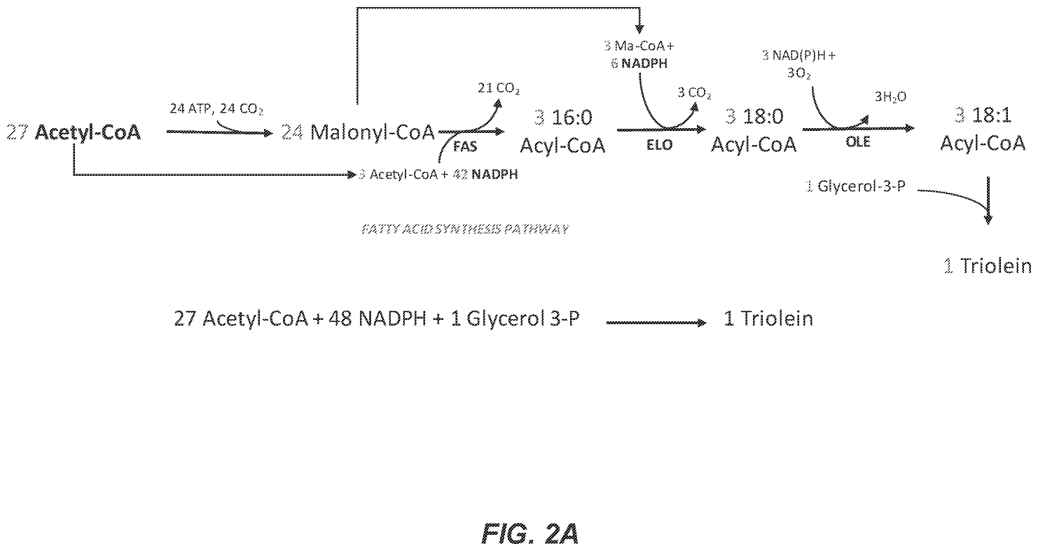

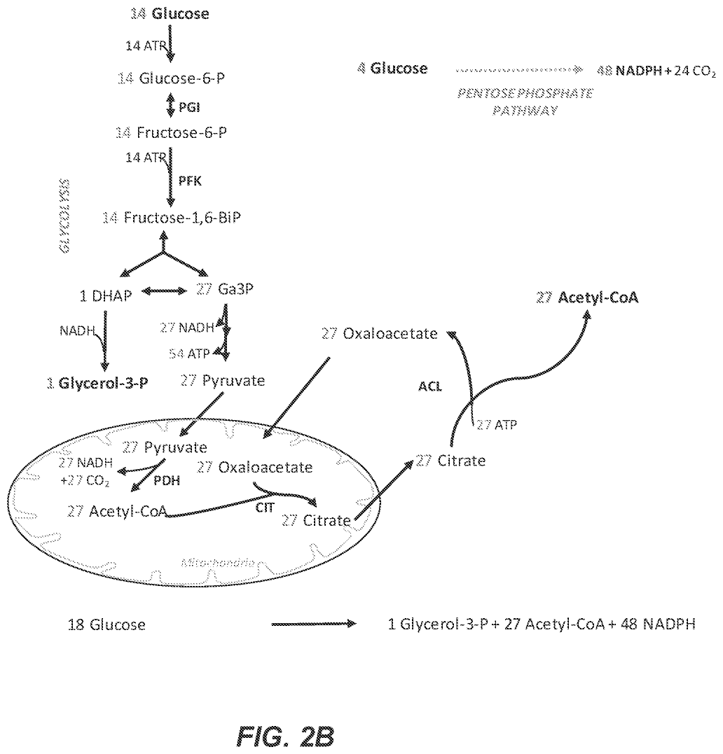

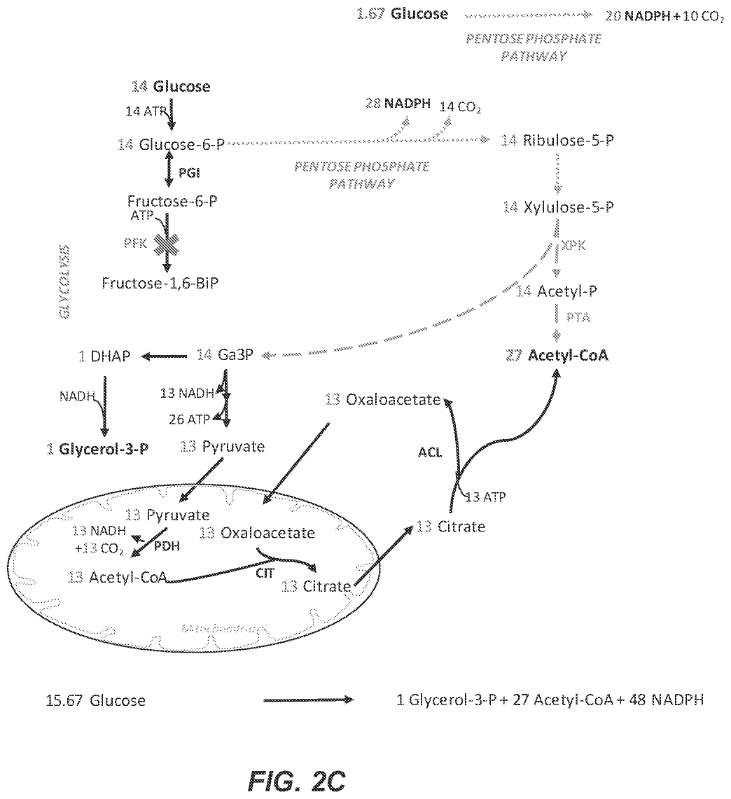

Phosphofructokinase (Pfk, EC 2.7.1.11) catalyzes the irreversible production of fructose 1,6-bisphosphate from fructose 6-phosphate (F6P). Carbon flux from glucose to lipids can still move through glycolysis in an otherwise unmodified Xpk/Pta strain whereas deleting PFK is expected to reroute glucose flux through the pentose phosphate pathway (PPP) [36,37] and in turn through the Xpk/Pta pathway (FIG. 1). Increased flux through the PPP could result in excess NADPH with negative consequences for growth [38,39], unless sufficient NADPH oxidizing reactions are present to restore the redox balance. It was hypothesized that introduction of the Xpk/Pta pathway into a pfk deficient Y. lipolytica strain would correct the NADPH imbalance by providing a route towards the NADPH-oxidizing lipid synthesis pathway (FIG. 1). The Xpk/Pta/Δpfk1 yeast cells described herein overcome the growth and lipid production deficits of the parent Δpfk1 strain. They also exhibit improved lipid yield and cell-specific lipid productivity over the wild-type.

### I. Definitions

The term “biologically-active portion” refers to an amino acid sequence that is less than a full-length amino acid sequence, but exhibits at least one activity of the full length sequence. For example, a biologically-active portion of a phosphoketolase may refer to one or more domains of a phosphoketolase having biological activity for converting xylulose-5-phosphate to glyceraldehyde-3-phosphate. Biologically-active portions of a protein include peptides or polypeptides comprising amino acid sequences sufficiently identical to or derived from the amino acid sequence of the protein, e.g., the amino acid sequence set forth in SEQ ID NOs: 28, 29, 30, or 31, which include fewer amino acids than the full length protein, and exhibit at least one activity (e.g., enzymatic activity, functional activity, etc.) of the protein. Similarly, biologically-active portions of a protein include peptides or polypeptides comprising amino acid sequences sufficiently identical to or derived from the amino acid sequence of the protein, e.g., an amino acid sequence set forth in SEQ ID NO:28, 29, 30, or 31, which include fewer amino acids than the full length protein, and exhibit at least one activity of the protein. A biologically-active portion of a protein may comprise, for example, at least 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700 or more amino acids, or any range or value derivable therein. Typically, biologically-active portions comprise a domain or motif having a catalytic activity, such as catalytic activity for producing a molecule in a fatty acid biosynthesis pathway, or having a transporter activity, such as for mitochondrial transport. A biologically-active portion of a protein includes portions of the protein that have the same activity as the full-length peptide and every portion that has more activity than background. For example, a biologically-active portion of an enzyme may have 0.1%, 0.5%, 1%, 2%, 3%, 4%, 5%, 10%, 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, 99.5%, 99.6%, 99.7%, 99.8%, 99.9%, 100%, 100.1%, 100.2%, 100.3%, 100.4%, 100.5%, 100.6%, 100.7%, 100.8%, 100.9%, 101%, 105%, 110%, 115%, 120%, 125%, 130%, 135%, 140%, 145%, 150%, 160%, 170%, 180%, 190%, 200%, 220%, 240%, 260%, 280%, 300%, 320%, 340%, 360%, 380%, 400% or higher activity relative to the full-length enzyme, or any range or value derivable therein. A biologically-active portion of a protein may include portions of a protein that lack a domain that targets the protein to a cellular compartment.

The term “exogenous” refers to anything that is introduced into a cell or has been introduced into a cell. An “exogenous nucleic acid” is a nucleic acid that entered a cell through the cell membrane. An “exogenous nucleic acid sequence” is a nucleic acid sequence of an exogenous nucleic acid. An exogenous nucleic acid may contain a nucleotide sequence that exists in the native genome of a cell and/or nucleotide sequences that did not previously exist in the cell's genome. Exogenous nucleic acids include exogenous genes. An “exogenous gene” is a nucleic acid that codes for the expression of an RNA and/or protein that has been introduced into a cell (e.g., by transformation/transfection), and is also referred to as a “transgene.” A cell comprising an exogenous nucleic acid may be referred to as a recombinant cell, into which additional exogenous gene(s) may be introduced. The exogenous gene may be from the same or different species relative to the cell being transformed. Thus, an exogenous gene can include a native gene that occupies a different location in the genome of the cell or is under different control, relative to the endogenous copy of the gene. An exogenous gene may be present in more than one copy in the cell. An exogenous gene may be maintained in a cell as an insertion into the genome (nuclear or plastid) or as an episomal molecule.

“In operable linkage” (or “operably linked”) refers to a functional linkage between two nucleic acid sequences, such a control sequence (typically a promoter) and the linked sequence (typically a sequence that encodes a protein, also called a coding sequence). A promoter is in operable linkage with a gene if it can mediate transcription of the gene.

The term “native” refers to the composition of a cell or parent cell prior to a transformation event. A “native gene” (also “endogenous gene”) refers to a nucleotide sequence that encodes a protein that has not been introduced into a cell by a transformation event. A “native protein” (also “endogenous protein”) refers to an amino acid sequence that is encoded by a native gene.

“Recombinant” refers to a cell, nucleic acid, protein, or vector, which has been modified due to introduction of an exogenous nucleic acid or alteration of a native nucleic acid. Resulting cells, nucleic acids, proteins or vectors are considered recombinant, as are progeny, offspring, duplications or replications of these are also considered recombinant. Thus, e.g., recombinant cells can express genes that are not found within the native (non-recombinant) form of the cell or express native genes differently than those genes are expressed by a non-recombinant cell Recombinant cells can, without limitation, include recombinant nucleic acids that encode for a gene product or for suppression elements such as mutations, knockouts, antisense, interfering RNA (RNAi), or dsRNA that reduce the levels of active gene product in a cell. A “recombinant nucleic acid” is derived from nucleic acid originally formed in vitro, in general, by the manipulation of nucleic acid, e.g., using polymerases, ligases, exonucleases, and endonucleases, or otherwise is in a form not normally found in nature. Once a recombinant nucleic acid is made and introduced into a host cell or organism, it may replicate using the in vivo cellular machinery of the host cell; however, such nucleic acids, once produced recombinantly, although subsequently replicated intracellularly, are still considered recombinant for purposes of this disclosure. Additionally, a recombinant nucleic acid refers to nucleotide sequences that comprise an endogenous nucleotide sequence and an exogenous nucleotide sequence; thus, an endogenous gene that has undergone recombination with an exogenous promoter is a recombinant nucleic acid. A “recombinant protein” is a protein made using recombinant techniques, i.e., through the expression of a recombinant nucleic acid.

“Transformation” refers to the transfer of a nucleic acid into a host organism or the genome of a host organism. Host organisms (and their progeny) containing the transformed nucleic acid fragments are referred to as “recombinant”, “transgenic” or “transformed” organisms. Thus, isolated polynucleotides of the present disclosure can be incorporated into recombinant constructs, typically DNA constructs, capable of introduction into and replication in a host cell. Such a construct can be a vector that includes a replication system and sequences that are capable of transcription and translation of a polypeptide-encoding sequence in a given host cell. Typically, expression vectors include, for example, one or more cloned genes under the transcriptional control of 5′ and 3′ regulatory sequences and a selectable marker. Such vectors also can contain a promoter regulatory region (e.g., a regulatory region controlling inducible or constitutive, environmentally- or developmentally-regulated, or location-specific expression), a transcription initiation start site, a ribosome binding site, a transcription termination site, and/or a polyadenylation signal. Alternatively, a cell may be transformed with a single genetic element, such as a promoter, which may result in genetically stable inheritance upon integrating into the host organism's genome, such as by homologous recombination.

The term “transformed cell” refers to a cell that has undergone a transformation. Thus, a transformed cell comprises the parent's genome and an inheritable genetic modification. Embodiments include progeny and offspring of such transformed cells.

The term “vector” refers to the means by which a nucleic acid can be propagated and/or transferred between organisms, cells, or cellular components. Vectors include plasmids, linear DNA fragments, viruses, bacteriophage, pro-viruses, phagemids, transposons, and artificial chromosomes, and the like, that may or may not be able to replicate autonomously or integrate into a chromosome of a host cell.

### II. Genetic Engineering

Vectors for transforming microorganisms (e.g., yeast cells) in accordance with the present disclosure can be prepared by known techniques familiar to those skilled in the art in view of the disclosure herein. A vector typically contains one or more genes, in which each gene codes for the expression of a desired product (the gene product) and is operably linked to one or more control sequences that regulate gene expression or target the gene product to a particular location in the recombinant cell.

Exogenous nucleic acid sequences, including, for example, nucleic acid sequences encoding a phosphoketolase protein and nucleic acid sequences encoding phosphotransacetylase protein, may be introduced into many different host cells. Nucleic acid sequences configured to facilitate a genetic mutation in a gene (e.g., a knockout mutation of a phosphofructokinase gene) may also be introduced into various host cells, as described further herein. Suitable host cells are microbial hosts that can be found broadly within the fungal families. Examples of suitable host strains include but are not limited to fungal or yeast species, such as Arxula, Aspegillus, Aurantiochytrium, Candida, Claviceps, Cryptococcus, Cunninghamella, Hansenula, Kluyveromyces, Leucosporidiella, Lipomyces, Mortierella, Ogataea, Pichia, Prototheca, Rhizopus, Rhodosporidium, Rhodotorula, Saccharomyces, Schizosaccharomyces, Tremella, Trichosporon, and Yarrowia. In some embodiments, a host cell of the present disclosure is Yarrowia lipolytica.

Microbial expression systems and expression vectors are well known to those skilled in the art. Any such expression vector could be used to introduce the instant genes and nucleic acid sequences into an organism. The nucleic acid sequences may be introduced into appropriate microorganisms via transformation techniques. For example, a nucleic acid sequence can be cloned in a suitable plasmid, and a parent cell can be transformed with the resulting plasmid. The plasmid is not particularly limited so long as it renders a desired nucleic acid sequence inheritable to the microorganism's progeny.

Vectors or cassettes useful for the transformation of suitable host cells are recognized in the art. Typically the vector or cassette contains a gene, sequences directing transcription and translation of a relevant gene including the promoter, a selectable marker, and sequences allowing autonomous replication or chromosomal integration. Suitable vectors comprise a region 5′ of the gene harboring the promoter and other transcriptional initiation controls and a region 3′ of the DNA fragment which controls transcriptional termination.

Promoters, cDNAs, and 3′UTRs, as well as other elements of the vectors, can be generated through cloning techniques using fragments isolated from native sources (Green & Sambrook, Molecular Cloning: A Laboratory Manual, (4th ed., 2012); U.S. Pat. No. 4,683,202; incorporated by reference). Alternatively, elements can be generated synthetically using known methods (Gene 164:49-53 (1995)).

### A. Vectors and Vector Components

Vectors for transforming microorganisms (e.g., yeast cells) in accordance with the present disclosure can be prepared by known techniques familiar to those skilled in the art in view of the disclosure herein. A vector typically contains one or more genes, in which each gene codes for the expression of a desired product (the gene product) and is operably linked to one or more control sequences that regulate gene expression or target the gene product to a particular location in the recombinant cell.

### 1. Control Sequences

Control sequences are nucleic acid sequences that regulate the expression of a coding sequence or direct a gene product to a particular location in or outside a cell. Control sequences that regulate expression include, for example, promoters that regulate transcription of a coding sequence and terminators that terminate transcription of a coding sequence. Another control sequence is a 3′ untranslated sequence located at the end of a coding sequence that encodes a polyadenylation signal. Control sequences that direct gene products to particular locations include those that encode signal peptides, which direct the protein to which they are attached to a particular location inside or outside the cell.

Thus, an exemplary vector design for expression of a gene in a cell (e.g., yeast cell) contains a coding sequence for a desired gene product (for example, a selectable marker, or an enzyme such as phosphoketoloase or phosphate acetytransferase) in operable linkage with a promoter active in the cell (e.g., in yeast). Alternatively, if the vector does not contain a promoter in operable linkage with the coding sequence of interest, the coding sequence can be transformed into the cells such that it becomes operably linked to an endogenous promoter at the point of vector integration.

The promoter used to express a gene can be the promoter naturally linked to that gene or a different promoter.

A promoter can generally be characterized as constitutive or inducible. Constitutive promoters are generally active or function to drive expression at all times (or at certain times in the cell life cycle) at the same level. Inducible promoters, conversely, are active (or rendered inactive) or are significantly up- or down-regulated only in response to a stimulus. Both types of promoters find application in the disclosed methods. Inducible promoters useful in the present disclosure include those that mediate transcription of an operably linked gene in response to a stimulus, such as an exogenously provided small molecule, temperature (heat or cold), lack of nitrogen in culture media, etc. Suitable promoters can activate transcription of an essentially silent gene or upregulate transcription of an operably linked gene that is transcribed at a low level.

Inclusion of termination region control sequence is optional. The termination region may be native to the transcriptional initiation region (the promoter), may be native to the DNA sequence of interest, or may be obtainable from another source (See, e.g., Chen & Orozco, Nucleic Acids Research 16:8411 (1988)).

### B. Genes and Codon Optimization

Typically, a gene includes a promoter, a coding sequence, and termination control sequences. When assembled by recombinant DNA technology, a gene may be termed an expression cassette and may be flanked by restriction sites for convenient insertion into a vector that is used to introduce the recombinant gene into a host cell. The expression cassette can be flanked by DNA sequences from the genome or other nucleic acid target to facilitate stable integration of the expression cassette into the genome by homologous recombination. Alternatively, the vector and its expression cassette may remain unintegrated (e.g., an episome), in which case, the vector typically includes an origin of replication, which is capable of providing for replication of the vector DNA.

A common gene present on a vector is a gene that codes for a protein, the expression of which allows the recombinant cell containing the protein to be differentiated from cells that do not express the protein. Such a gene, and its corresponding gene product, is called a selectable marker or selection marker. Any of a wide variety of selectable markers can be employed in a transgene construct useful for transforming the organisms of the invention.

For optimal expression of a recombinant protein, it may be beneficial to employ coding sequences that produce mRNA with codons optimally used by the host cell to be transformed. Thus, proper expression of transgenes can require that the codon usage of the transgene matches the specific codon bias of the organism in which the transgene is being expressed. The precise mechanisms underlying this effect are many, but include the proper balancing of available aminoacylated tRNA pools with proteins being synthesized in the cell, coupled with more efficient translation of the transgenic messenger RNA (mRNA) when this need is met. When codon usage in the transgene is not optimized, available tRNA pools may not be sufficient to allow for efficient translation of the transgenic mRNA resulting in ribosomal stalling and termination and possible instability of the transgenic mRNA.

An XPK or PTA coding sequence of the present disclosure can be codon optimized for a particular host cell by replacing one or more rare codons with one or more codons more frequently found in the host cell. A rare codon in a host cell describes a codon that is found in less than 1%, less than 2%, less than 5%, less than 10%, or less than 20% of coding sequences in the host cell. Non-limiting examples of rare codons for Yarrowia lipolytica are TTA, ATA, and AGG. These can be replaced, for example, with CTG, ATC, and CGA respectively. Non-limiting examples of rare codons for Saccharomyces cerevisiae are CGC, CGA, and TCG. These can be replaced with, for example, AGA, AGA, and TCT respectively. Non-limiting examples of rare codons for Arxula adeninivorans are ATA, CTA, and AGT. These can be replaced with, for example, ATT, CTG, and TCT respectively. Rare codons can be identified using methods known to those of skill in the art, for example as discussed in the Examples.

Aspects of the disclosure comprise transformation of a microorganism with a nucleic acid sequence comprising a gene that encodes a protein. The gene may be native to the cell or from a different species. The gene may be derived from a different species yet modified (e.g., codon optimized) for optimal expression in the microorganism. In certain embodiments, the gene is inheritable to the progeny of a transformed cell. In some embodiments, the gene is inheritable because it resides on a plasmid. In certain embodiments, the gene is inheritable because it is integrated into the genome of the transformed cell.

Further aspects of the disclosure may comprise transformation of a microorganism with a nucleic acid sequence configured to generate a mutation in a gene of the microorganism. For example, aspects of the disclosure may comprise transformation of the microorganism with a nucleic acid sequence comprising sequences upstream and downstream of a gene (e.g., a phosphofructokinase gene), thereby facilitating reduced expression or deletion of the gene via homologous recombination. Various methods for generating mutations (including deletions or knockout mutations, as well as mutations which reduce expression of a gene) in genes of a microorganism are recognized in the art and envisioned herein. A microorganism having a deletion or knockout mutation of a gene does not product a functional copy of the protein. For example, a recombinant yeast cell of the disclosure may comprise a deletion of an endogenous phosphofructokinase gene, such that the recombinant yeast cell does not express an endogenous phosphofructokinase protein. A microorganism having a reduced expression of a gene or protein produces a functional copy of the protein, but at a reduced amount compared with a wild-type (i.e., a non-recombinant or non-genetically modified) microorganism of the same species. Methods for reducing expression of a protein are recognized in the art and include, for example, replacement of an endogenous promoter and/or modification of one or more regulatory elements.

### C. Transformation

Cells can be transformed by any suitable technique including, e.g., biolistics, electroporation, glass bead transformation, and silicon carbide whisker transformation. Any convenient technique for introducing a transgene into a microorganism can be employed in the present invention. Transformation can be achieved by, for example, the method of D. M. Morrison (Methods in Enzymology 68:326 (1979)), the method by increasing permeability of recipient cells for DNA with calcium chloride (Mandel & Higa, J. Molecular Biology, 53:159 (1970)), or the like.

Examples of expression of transgenes in oleaginous yeast (e.g., Yarrowia lipolytica) can be found in the literature (Bordes et al., J. Microbiological Methods, 70:493 (2007); Chen et al., Applied Microbiology & Biotechnology 48:232 (1997)).

Vectors for transformation of microorganisms in accordance with the present invention can be prepared by known techniques familiar to those skilled in the art. In one embodiment, an exemplary vector design for expression of a gene in a microorganism contains a gene encoding an enzyme in operable linkage with a promoter active in the microorganism. Alternatively, if the vector does not contain a promoter in operable linkage with the gene of interest, the gene can be transformed into the cells such that it becomes operably linked to a native promoter at the point of vector integration. The vector can also contain a second gene that encodes a protein. Optionally, one or both gene(s) is/are followed by a 3′ untranslated sequence containing a polyadenylation signal. Expression cassettes encoding the two genes can be physically linked in the vector or on separate vectors. Co-transformation of microbes can also be used, in which distinct vector molecules are simultaneously used to transform cells (Protist 155:381-93 (2004)). The transformed cells can be optionally selected based upon the ability to grow in the presence of the antibiotic or other selectable marker under conditions in which cells lacking the resistance cassette would not grow.

### D. Genetically Engineered Cells

Aspects of the disclosure comprise genetically engineered cells and methods for making and using such cells. In some embodiments, disclosed are recombinant cells comprising one or more exogenous nucleic acid sequences. Also disclosed are methods for generating such recombinant cells comprising introducing the one or more exogenous nucleic acid sequences into a host cell. Further described are methods for collecting one or more products (e.g., a lipid, an oil, etc.) from such recombinant cells comprising culturing the cells and collecting the product. In some embodiments, a recombinant cell of the disclosure is a bacterial cell (e.g. E. coli), a fungal cell, a yeast cell, or a plant cell.

In some embodiments, a recombinant cell of the disclosure is a recombinant yeast

cell. The yeast cell may be selected from the group consisting of Arxula, Aspegillus, Aurantiochytrium, Candida, Claviceps, Cryptococcus, Cunninghamella, Geotrichum, Hansenula, Kluyveromyces, Kodamaea, Leucosporidiella, Lipomyces, Mortierella, Ogataea, Pichia, Prototheca, Rhizopus, Rhodosporidium, Rhodotorula, Saccharomyces, Schizosaccharomyces, Tremella, Trichosporon, Wickerhamomyces, and Yarrowia.

In some embodiments, the yeast cell is selected from the group of consisting of Arxula adeninivorans, Aspergillus niger, Aspergillus orzyae, Aspergillus terreus, Aurantiochytrium limacinum, Candida utilis, Claviceps purpurea, Cryptococcus albidus, Cryptococcus curvatus, Cryptococcus ramirezgomezianus, Cryptococcus terreus, Cryptococcus wieringae, Cunninghamella echinulata, Cunninghamella japonica, Geotrichum fermentans, Hansenula polymorpha, Kluyveromyces lactis, Kluyveromyces marxianus, Kodamaea ohmeri, Leucosporidiella creatinivora, Lipomyces lipofer, Lipomyces starkeyi, Lipomyces tetrasporus, Mortierella isabellina, Mortierella alpina, Ogataea polymorpha, Pichia ciferrii, Pichia guilliermondii, Pichia pastoris, Pichia stipites, Prototheca zopfii, Rhizopus arrhizus, Rhodosporidium babjevae, Rhodosporidium toruloides, Rhodosporidium paludigenum, Rhodotorula glutinis, Rhodotorula mucilaginosa, Saccharomyces cerevisiae, Schizosaccharomyces pombe, Tremella enchepala, Trichosporon cutaneum, Trichosporon fermentans, Wickerhamomyces ciferrii, and Yarrowia lipolytica.

In certain embodiments, the yeast cell is Saccharomyces cerevisiae, Yarrowia lipolytica, or Arxula adeninivorans. In some embodiments, the yeast cell is Saccharomyces cerevisiae. In some embodiments, the yeast cell is Arxula adeninivorans. In some embodiments, the yeast cell is Yarrowia lipolytica.

A recombinant cell of the present disclosure can include one or more modifications to the native lipid biosynthetic pathway enzymes or coding sequences for increased lipid production. The cell can include one or more copies of an exogenous nucleic acid sequence encoding a phosphoketolase protein and one or more copies of an exogenous nucleic acid sequence encoding a phosphotransacetylase protein in combination with one more native lipid pathway modifications for increased lipid production. Such lipid pathway modifications include, for example, (1) up-regulation of DGA1, DGA2, ACC1, or OLE1 genes, or any combination thereof; (2) down-regulation of TGL3, TGL4, or POX1-6 genes, or any combination thereof; (3) one or more substitutions or deletions in the coding or noncoding sequences of genes involved in the lipid biosynthetic pathway.

In some embodiments, disclosed is a recombinant yeast cell comprising an exogenous nucleic acid sequence encoding a phosphoketolase protein. In some embodiments, the phosphoketolase protein is a phosphoketolase protein from a bacterium of the genus Clostridium. In some embodiments, the phosphoketolase protein is a phosphoketolase protein from Clostridium carboxidivorans or Clostridium viride. In some embodiments, the phosphoketolase protein is a phosphoketolase protein from Clostridium acetobutylicum. In some embodiments, the recombinant yeast cell comprises an exogenous nucleic acid sequence encoding a phosphotransacetylase protein. In some embodiments, the phosphotransacetylase protein is a phosphotransacetylase protein from a bacterium of the genus Bacillus. In some embodiments, the phosphotransacetylase protein is a phosphotransacetylase protein from Bacillus subtilis. In some embodiments, the phosphotransacetylase protein is a phosphotransacetylase protein from a bacterium of the genus Thermoanaerobacterium. In some embodiments, the phosphotransacetylase protein is a phosphotransacetylase protein from Thermoanaerobacterium saccharolyticum. In some embodiments, the recombinant yeast cell has reduced expression of an endogenous phosphofructokinase protein compared to a wild-type yeast cell. The recombinant yeast cell may have, have at least, or have at most about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% reduced expression of an endogenous phosphofructokinase protein compared to a wild-type yeast cell, or any range or value derivable therein. In some embodiments, the recombinant yeast cell does not express an endogenous phosphofructokinase protein. In some embodiments, the recombinant yeast cell expresses an endogenous phosphofructokinase gene.

### III. Nucleic Acids and Proteins

Aspects of the present disclosure are directed to nucleic acid sequences, including gene sequences, encoding for one or more proteins and recombinant cells comprising such sequences. Recombinant cells of the disclosure may comprise at least 1, 2, 3, or more nucleic acid sequences described herein. In some embodiments, a recombinant cell comprises a first exogenous nucleic acid sequence encoding a phosphoketolase protein and a second exogenous nucleic acid sequence encoding a phosphotransacetylase protein.

In some embodiments, a nucleic acid sequence of the disclosure is a sequence encoding a phosphoketolase (“Xpk” or “XPK”) protein. Three versions of phosphoketolase exist, those acting on the five carbon xylulose-5-phosphate (X-5-P), EC 4.1.2.9, those acting on the six carbon fructose-6-phosphate (F-6-P), EC 4.1.2.22, and those with bifunctional activity on both substrates. The 6-carbon phosphoketolase together with a transketolase (EC 2.2.1.1), which is present in all microorganisms, catalyze reactions with the same net conversion of xylulose-5-phosphate to acetyl phosphate (Ac-P) and glyceraldehyde-3-phosphate (Ga-3-P) as the 5-carbon phosphoketolase. Transketolase coverts xylulose-5-phosphate (X-5-P) and erythrose-4-phosphate (E-4-P) to fructose-6-phosphate (F-6-P) and glyceraldehyde-3-phosphate (Ga-3-P).

EC 4.1.2.9 X-5-P+Pi->Ac-P+Ga-3-P+H2O i)

EC 4.1.2.22 F-6-P+Pi->Ac-P+E-4-P+H2O ii)

EC 2.2.1.1 X-5-P+E-4-P->F-6-P+Ga-3-P iii)

Net: X-5-P+Pi->Ac-P+Ga-3-P+H2O

The phosphoketolase protein may be classified by Enzyme Commission number EC 4.1.2.9. The phosphoketolase protein may be classified by Enzyme Commission number EC 4.1.2.22.

In some embodiments, the phosphoketolase protein is a phosphoketolase protein from a bacterium of the genus Clostridium. In some embodiments, the phosphoketolase protein is a phosphoketolase protein from Clostridium carboxidivorans or Clostridium viride. In some embodiments, the phosphoketolase protein is a phosphoketolase protein from Clostridium acetobutylicum. A phosphoketolase protein from Clostridium acetobutylicum describes a protein from Clostridium acetobutylicum having phosphoketolase activity. In some embodiments, a phosphoketolase protein of the present disclosure comprises an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or more sequence identity with SEQ ID NO:28, or any range or value derivable therein. The phosphoketolase protein may be substantially identical to SEQ ID NO:28, and retain the functional activity of the protein of SEQ ID NO:28, yet differ in amino acid sequence, e.g., due to either natural allelic variation or mutagenesis. In some embodiments, the phosphoketolase protein comprises SEQ ID NO:28.

In some embodiments, the nucleic acid sequence is a natural gene sequence encoding a Clostridium acetobutylicum phosphoketolase protein. In some embodiments, the nucleic acid sequence is a codon optimized sequence encoding a Clostridium acetobutylicum phosphoketolase protein. In some embodiments, the nucleic acid sequence is at least, is, or is at most 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or more identical to SEQ ID NO:26, or any range or value derivable therein. In some embodiments, the nucleic acid sequence comprises SEQ ID NO:26. In some embodiments, the nucleic acid sequence is SEQ ID NO:26. In some embodiments, the nucleic acid sequence comprises 1, 2, 3, 4, 5, 6, 7, 8, 9, 10, or more copies of SEQ ID NO:26. In some embodiments, the nucleic acid sequence is at least, is, or is at most 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or more identical to SEQ ID NO:27, or any range or value derivable therein. In some embodiments, the nucleic acid sequence comprises SEQ ID NO:27. In some embodiments, the nucleic acid sequence is SEQ ID NO:27. In some embodiments, the nucleic acid sequence comprises 1, 2, 3, 4, 5, 6, 7, 8, 9, 10, or more copies of SEQ ID NO:27.

In some embodiments, a nucleic acid sequence of the disclosure is a sequence encoding a phosphotransacetylase (also known as “phosphate acetyltransferase”) protein (“Pta” or “PTA”). A phosphotransacetylase protein describes a protein capable of catalyzing the reversible conversion of AcP to acetyl-CoA and is classified by Enzyme Commission number EC 2.3.1.8.

In some embodiments, the phosphotransacetylase protein is a phosphotransacetylase protein from a bacterium of the genus Bacillus. In some embodiments, the phosphotransacetylase protein is a phosphotransacetylase protein from Bacillus subtilis. A phosphotransacetylase protein from Bacillus subtilis describes a protein from Bacillus subtilis having phosphotransacetylase activity. In some embodiments, a phosphotransacetylase protein of the present disclosure comprises an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or more sequence identity with SEQ ID NO:29, or any range or value derivable therein. The phosphotransacetylase protein may be substantially identical to SEQ ID NO:29, and retain the functional activity of the protein of SEQ ID NO:29, yet differ in amino acid sequence, e.g., due to either natural allelic variation or mutagenesis. The phosphotransacetylase protein may comprise SEQ ID NO:29.

In some embodiments, the phosphotransacetylase protein is a phosphotransacetylase protein from a bacterium of the genus Thermoanaerobacterium. In some embodiments, the phosphotransacetylase protein is a phosphotransacetylase protein from Thermoanaerobacterium saccharolyticum. A phosphotransacetylase protein from Thermoanaerobacterium saccharolyticum describes a protein from Thermoanaerobacterium saccharolyticum having phosphotransacetylase activity. In some embodiments, a phosphotransacetylase protein of the present disclosure comprises an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or more sequence identity with SEQ ID NO:30, or any range or value derivable therein. The phosphotransacetylase protein may be substantially identical to SEQ ID NO:30, and retain the functional activity of the protein of SEQ ID NO:30, yet differ in amino acid sequence, e.g., due to either natural allelic variation or mutagenesis. The phosphotransacetylase protein may comprise SEQ ID NO:30.

In some embodiments, a nucleic acid sequence of the disclosure is a sequence configured to facilitate deletion of at least a portion of an endogenous phosphofructokinase (pfk) gene in a cell. Methods for making and using nucleic acid sequences configured to facilitate deletion of at least a portion of a gene are recognized in the art and include, for example, the methods described in U.S. Pat. No. 10,760,105, incorporated by reference herein in its entirety. Such a deletion will result in a cell that does not express an endogenous phosphofructokinase protein. Also contemplated are methods for modifying the expression of a phosphofructokinase protein, for example, by facilitating reduced expression or activity of a phosphofructokinase protein during a lipid accumulation phase of Yarrowia lipolytica relative to a growth phase; such methods are described in, for example, U.S. Patent Publication No. 2021/0032604, incorporated herein by reference in its entirety. The phosphofructokinase protein may be classified by Enzyme Commission number EC 2.7.1.11. In some embodiments, the phosphofructokinase protein is Yarrowia lipolytica phosphofructokinase (encoded by the gene pfk1).

In some embodiments, the nucleic acid sequence comprises a portion of an endogenous phosphofructokinase gene. In some embodiments, the nucleic acid sequence comprises a sequence corresponding to a region upstream and/or downstream of an endogenous phosphofructokinase gene (e.g., a region comprising least 10, 20, 30, 40, 50, 60, 70, 80, 90, 100, 200, 300, 400, 500, 600, 700, 800, 900, 1000, or more nucleotides starting at least 0, 1, 2, 3, 4, 5, 10, 20, 30, 40, 50, 60, 70, 80, 90, 100, 150, 200, upstream or downstream from the endogenous phosphofructokinase gene). In some embodiments, the nucleic acid sequence comprises a sequence corresponding to nucleotides −636 to −187 upstream of Yarrowia lipolytica pfk1 (SEQ ID NO:7). In some embodiments, the nucleic acid sequence comprises a sequence corresponding to the 621 nucleotides immediately downstream of Yarrowia lipolytica pfk1 (SEQ ID NO:8).

Also contemplated herein are fusion proteins. A fusion protein describes a polypeptide comprising two or more proteins expressed together as a single polypeptide sequence. In some embodiments, a fusion protein comprises a phosphoketolase protein and a phosphotransacetylase protein. In some embodiments, a fusion protein comprises a phosphoketolase protein and a phosphotransacetylase protein attached via a linker (e.g., a peptide linker).

### IV. Products

In some aspects, the present disclosure relates to a method of producing a product, comprising providing a genetically modified cell (e.g., a recombinant yeast cell), and culturing the cell for a period of time on a substrate, thereby producing the product. In some embodiments, the method further comprises generating the genetically modified cell, for example by transforming a cell (e.g., a yeast cell) with one or more exogenous nucleic acids encoding one or more proteins (e.g., phosphoketolase and/or phosphotransacetylase).

The substrate may comprise depolymerized sugar beet pulp, glycerin, black liquor, corn, corn starch, corn dextrins, depolymerized cellulosic material, corn stover, sugar beet pulp, switchgrass, milk whey, molasses, potato, rice, sorghum, sugar cane, thick cane juice, sugar beet juice, and/or wheat. In certain embodiments, the transformed cells are grown in the presence of exogenous fatty acids, glucose, ethanol, xylose, sucrose, starch, starch dextrin, glycerol, cellulose, and/or acetic acid. These compounds may be added to the substrate during cultivation to increase lipid production. The exogenous fatty acids may include stearate, oleic acid, linoleic acid, γ-linolenic acid, dihomo-γ-linolenic acid, arachidonic acid, α-linolenic acid, stearidonic acid, eicosatetraenoic acid, eicosapenteaenoic acid, docosapentaenoic acid, eicosadienoic acid, and/or eicosatrienoic acid.

In certain embodiments, the present disclosure relates to a product produced by a recombinant (also “genetically modified”) yeast cell described herein. In some embodiments, the product is an acetyl-CoA derived product. As used herein, an “acetyl-CoA derived product,” describes any product generated by a cell using acetyl-CoA as a precursor. In certain embodiments, the product is an oil, lipid, fatty acid, fatty alcohol, triacylglyceride, terpene, isoprenoid, or farnesene. In some embodiments, the product is stearic acid, oleic acid, linoleic acid, capric acid, caprylic acid, caproic acid, lauric acid, myristic acid, palmitic acid, palmitoleic acid, or squalene. In certain embodiments, the product is a saturated fatty acid. Thus, the product may be caprylic acid, capric acid, lauric acid, myristic acid, palmitic acid, stearic acid, arachidic acid, behenic acid, lignoceric acid, or cerotic acid. In some embodiments, the product is an unsaturated fatty acid. Thus, the product may be myristoleic acid, palmitoleic acid, sapienic acid, oleic acid, elaidic acid, vaccenic acid, linoleic acid, linoelaidic acid, α-linolenic acid, arachidonic acid, eicosapenteaenoic acid, erucic acid, or docosahexaenoic acid.

In some embodiments, the product comprises an 18-carbon fatty acid. In some embodiments, the product comprises oleic acid, stearic acid, or linoleic acid. In some embodiments, the product is oleic acid. In some embodiments, the product is stearic acid. In some embodiments, the product is linoleic acid.

In some embodiments, the method comprises collecting the product. The method may comprise purifying the product, e.g., separating one or more lipid fractions from a culture of genetically modified cells from one or more aqueous fractions of the culture.

### EXAMPLES

The following examples are included to demonstrate certain embodiments of the invention. It should be appreciated by those of skill in the art that the techniques disclosed in the examples which follow represent techniques discovered by the inventor to function well in the practice of the invention, and thus can be considered to constitute certain modes for its practice. However, those of skill in the art should, in light of the present disclosure, appreciate that many changes can be made in the specific embodiments which are disclosed and still obtain a like or similar result without departing from the spirit and scope of the invention.

### Example 1—Identification of Functional Heterologous Phosphotransacetylase (PTA) and Phosphoketolase (XPK) in Y. lipolytica

Heterologous PTA and XPK genes from various species of bacteria, archaea, algae and fungi were tested for activity in Y. lipolytica.

Genes were individually expressed in wild-type strain YB-392 and cell-free extracts were assayed to measure phosphotransacetylase (Pta) and phosphoketolase (Xpk) activity. Lists of all the PTA and XPK genes tested are shown in Tables 1 and 2.

TABLE 1
List of PTA genes tested in Y. lipolytica

| | | Variants |
|---|---|---|
| Donor Organism | Accession # | tested |
| Methanosarcina thermophila | P38503 | Native |
| Methanosarcina barkeri | Q46BI0 | Native |
| Methanosarcina acetivorans | Q8TK18 | Native |
| Bacillus subtilis | P39646 | Native |
| | | CO to S. cerevisiae |
| | | CO to Y. lipolytica |
| Clostridium acetobutylicum | P71103 | Native |
| Thermoanaerobacterium | I3VW55 | Native |
| saccharolyticum | | CO to S. cerevisiae |
| | | CO to Y. lipolytica |
| Aphanomyces astaci | W4HBJ2 | CO to S. cerevisiae |
| Aphanomyces invadans | A0A024UQB7 | CO to S. cerevisiae |
| Auxenochlorella protothecoides | A0A087SL07 | CO to S. cerevisiae |
| Beauveria bassiana | A0A0A2W136 | CO to S. cerevisiae |
| Chlamydomonas reinhardtii | A8IQQ1 | CO to S. cerevisiae |
| Guillardia theta | L1JP48 | CO to S. cerevisiae |
| Helicosporidium sp. | A0A059LKK9 | CO to S. cerevisiae |
| Perkinsus marinus | C5LE15 | CO to S. cerevisiae |
| Phytophthora parasitica | W3A4X6 | CO to S. cerevisiae |
| Phytophthora ramorum | H3GP49 | CO to S. cerevisiae |
| Phytophthora sojae | G4ZCU5 | CO to S. cerevisiae |
| Pythium ultimum | K3X6T0 | CO to S. cerevisiae |
| Saprolegnia diclina | T0PRY0 | CO to S. cerevisiae |
| Selaginella moellendorffii | D8STZ3 | CO to S. cerevisiae |
| Volvox carteri | D8TQ82 | CO to S. cerevisiae |

CO—codon optimized

TABLE 2
List of XPK genes tested in Y. lipolytica

| Donor Organism | Accession | Variants tested |
|---|---|---|
| Rhodosporidium toruloides | M7WGA7 | Native |
| | | CO to S. cerevisiae |
| Aspergillus niger | A2QDB0 | Native |
| | | CO to S. cerevisiae |
| Penicillium chrysogenum | B6HBY3 | Native |
| Trichoderma reesei | A0A024S8D5 | Native |
| | | CO to S. cerevisiae |
| Lactobacillus curvatus | EHE86779 | Native |
| | | CO to S. cerevisiae |
| | | CO to Y. lipolytica |
| Aspergillus nidulans | C8V9F5 | Native |
| | | CO to S. cerevisiae |
| Bifidobacterium adolescentis | Q6R2R0 | CO to S. cerevisiae |
| Leuconostoc mesenteroides | A0A5M8XD05 | CO to S. cerevisiae |
| Lactococcus lactis | Q9CFH4 | CO to S. cerevisiae |
| Lactobacillus graminis | A0A5J6Z6A6 | CO to S. cerevisiae |
| Lactobacillus fuchuensis | A0A0R1RRQ4 | CO to S. cerevisiae |
| Lactobacillus concavus | A0A0R1VX71 | CO to S. cerevisiae |
| Lactobacillus dextrinicus | A0A0R2BTA3 | CO to S. cerevisiae |
| | | CO to Y. lipolytica |
| Lactobacillus ceti | A0A0R2KG92 | CO to S. cerevisiae |
| | | CO to Y. lipolytica |
| Bifidobacterium breve | D4BNU9 | CO to S. cerevisiae |
| Leuconostoc lactis | KQB80457 | CO to S. cerevisiae |
| Oenococcus oeni | A0NKP8 | CO to S. cerevisiae |
| Clostridium acetobutylicum | Q97JE3 | CO to S. cerevisiae |
| | | CO to Y. lipolytica |
| Clostridium carboxidivorans | C6PNC7 | CO to S. cerevisiae |
| Streptococcus pantholopis | A0A172Q961 | CO to S. cerevisiae |
| Streptococcus thermophilus | WP_049554686 | CO to S. cerevisiae |
| | | CO to Y. lipolytica |

CO—codon optimized

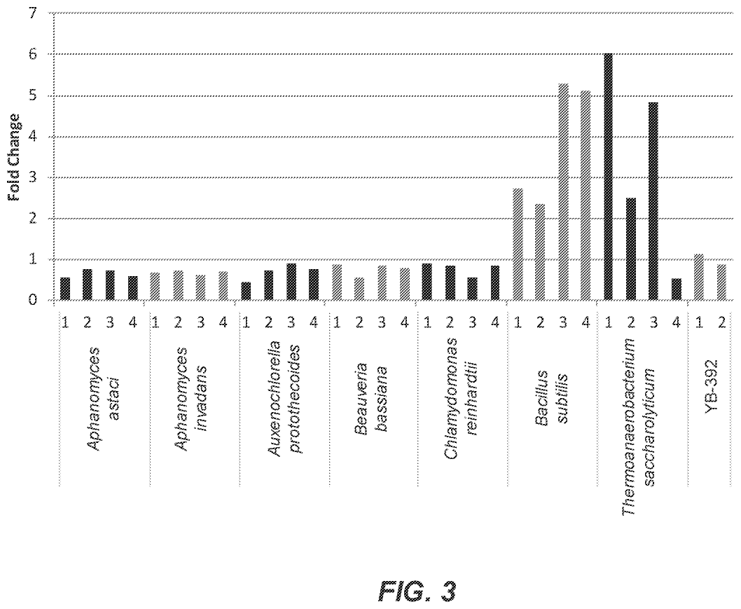

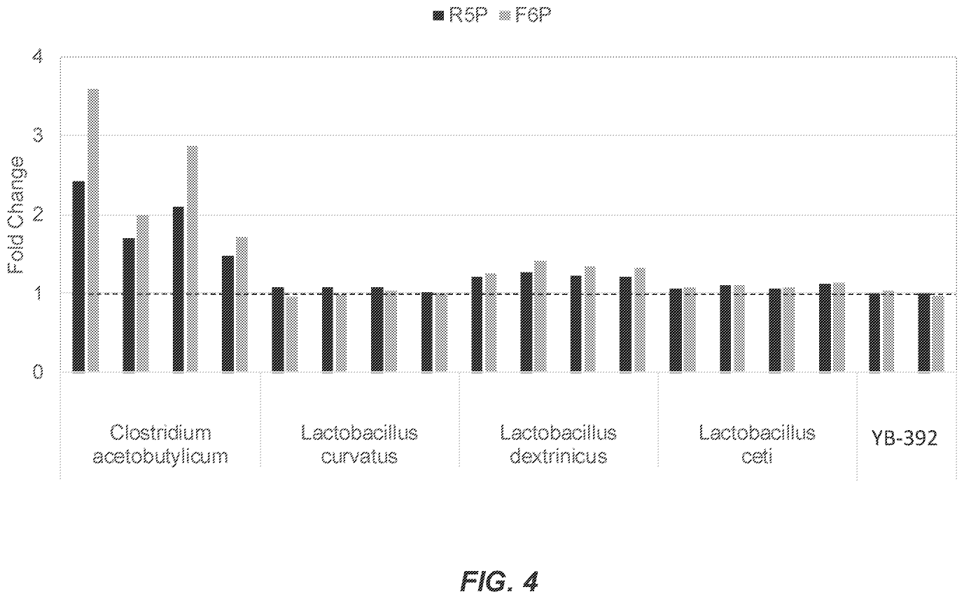

Leuconostoc mesenteroides Xpk and Clostridium kluyveri Pta were previously expressed in the Y. lipolytica PO1g lineage [28]. Xpk and Pta activity was not reported in that study and L. mesenteroides XPK did not exhibit activity in the wild-type strain YB-392 used here. The wild-type strain contains no endogenous Xpk or Pta and shows background levels of activity. Strains expressing PTA genes from Bacillus subtilis (BsPTA(v1)) and Thermoanaerobacterium saccharolyticum and an XPK gene from Clostridium acetobutylicum (CaXPK(v1)) exhibited the highest activity in these screens (FIGS. 3 and 4).

In the course of identifying active genes in Y. lipolytica, different methods of screening were successful for PTA and XPK. Linear integrating expression cassettes and codon optimization to S. cerevisiae resulted in successful PTA expression. This approach did not yield detectable Xpk activity in these studies, presumably due to low expression from the candidate genes. Codon optimization of XPK genes to Y. lipolytica and expression from replicating plasmids, a method of expression that yields uniform high expression, was required to identify one gene candidate with detectable enzymatic activity (FIG. 4).

### Methods

### Strains, Cultivation and Media

Wild-type, haploid Yarrowia lipolytica strain YB-392 was obtained from the ARS Culture Collection (NRRL). For routine growth and genetic transformation, strains were cultured in YPD (10 g/L yeast extract, 20 g/L bacto peptone, 20 g/L glucose), YPD/Et/Gly (YPD as described, plus 20 g/L ethanol and 30 g/L glycerol) and YPG (10 g/L yeast extract, 20 g/L bacto peptone, 20 g/L glycerol) media at 30° C. 20 g/L agar was added to prepare solid media. Selection was performed using 300 μg/mL hygromycin B (Corning), 500 μg/mL nourseothricin (Werner Bioreagents) or 30 μM 5-fluoro-2′-deoxyuridine (FUDR) (Fisher Scientific).

### Example 2—Construction and Characterization of the Y. lipolytica Δpfk1 Strain (NS1047)

To increase glucose flux through the Xpk/Pta pathway, the PFK1 gene was deleted to partially disable glycolysis (FIG. 1). Y. lipolytica contains only one PFK gene (YALI0D16357) and its deletion leads to loss of growth on minimal media with glucose as the only carbon source [41]. The PFK1 gene was deleted in YB-392 to create the Δpfk1 (pfk1 knockout) strain, designated NS1047.

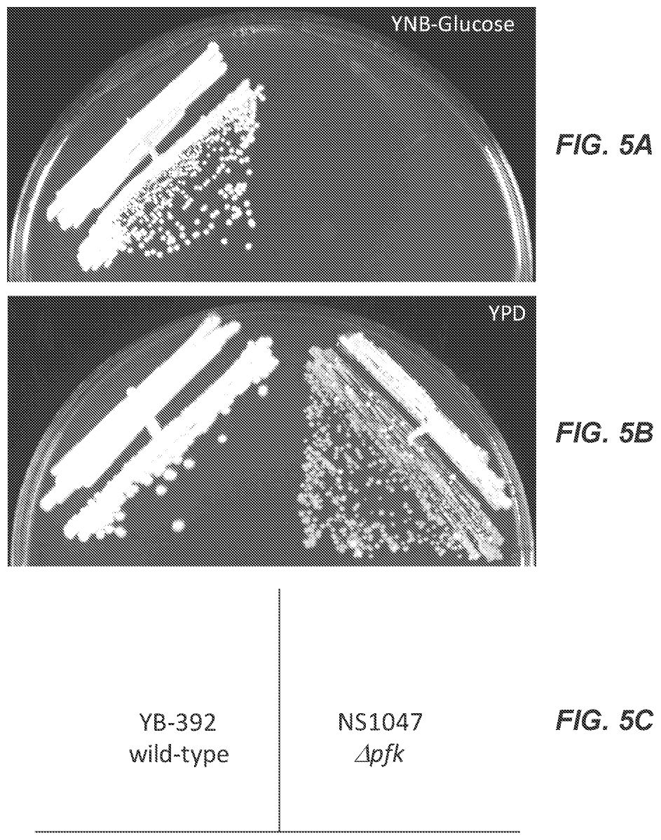

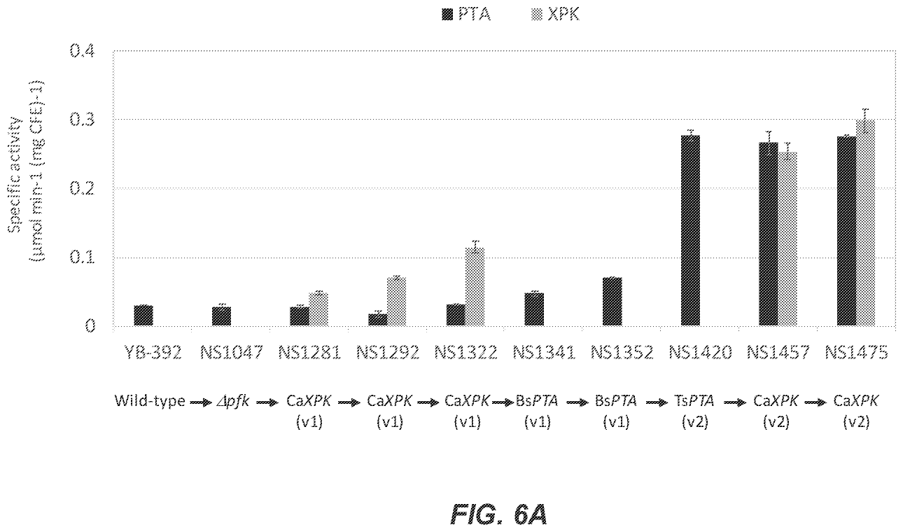

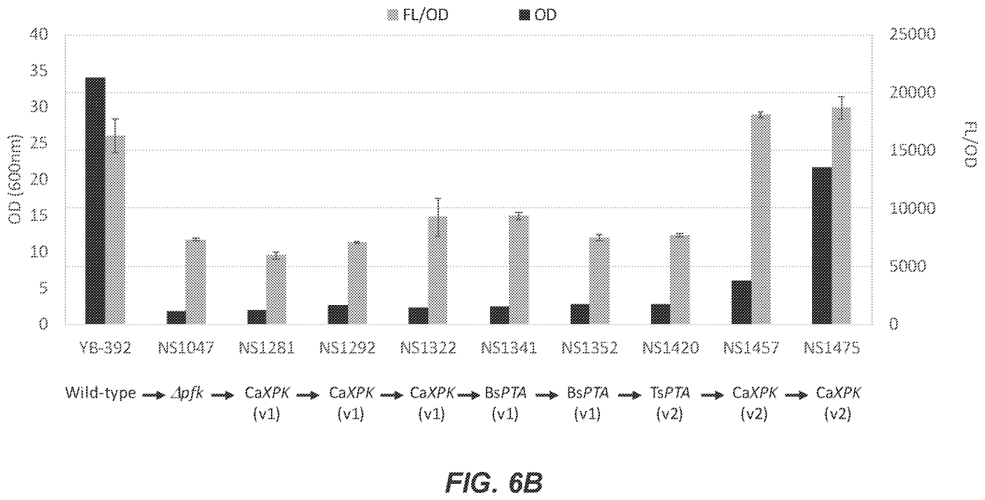

Disruption of glycolysis through PFK or PGI deletions can cause growth defects on glucose [36,38], presumably due to excess NADPH produced by increased glucose flux through the PPP [39,42]. In organisms that can metabolize cytosolic NADPH, glycolytic deficient strains retain or regain growth on glucose (e.g., through native cytosolic NADPH oxidase in Kluyveromyces lactis or by overexpression of a transhydrogenase enzyme in E. coli [42]). Y. lipolytica lacks cytosolic NADPH oxidase and homologues to PFK1 [41]. NS1047 was unable to grow in minimal glucose media (FIG. 5A) and lipid production media (FIG. 6B, black bar) with glucose as the primary carbon source. It was therefore hypothesized that Y. lipolytica cannot tolerate the excess NADPH produced when glucose is consumed in a PFK-deleted strain. Growth on YPD was not abolished but it was slower for NS1047 than YB-392 (FIG. 5B). The reduced severity of the growth defect on this rich medium could be attributed to the ability of Y. lipolytica to utilize amino acids for growth and the ability of a pfk1 deletion mutant to metabolize permissive carbon sources in the presence of glucose [41].

### Methods

### PFK1 Deletion

The Y. lipolytica PFK1 gene YALIOD16357 was deleted through targeted genomic integration using direct repeats and a combination of positive and negative selection for marker recycling. Using standard molecular biology techniques, a construct was designed comprising of the genetic parts listed in Table 3. A two-fragment deletion cassette was amplified by PCR using a combination of terminal and internal oligonucleotide primers such that the fragments overlapped in the nat marker reading frame, but neither fragment alone contained the entire functional nourseothricin-resistance gene. PCR products were transformed into hydroxyurea-treated cells as described in previously [49]. Transformation recovery was in YPD/Et/Gly to provide carbon sources in addition to glucose. Transformed cells were plated on YPD/Et/Gly containing 500 μg/mL nourseothricin. Successful cassette integration replaced the PFK1 locus by a double recombination event at the 47-bp upstream and 621-bp downstream regions. A longer downstream homology region was chosen to increase the likelihood of this recombination event as opposed to recombination between the homologous 450-bp regions in the integration cassette and upstream of PFK1. Nourseothricin-resistant colonies were screened by PCR for the presence of the expected targeted integration product and the absence of the PFK1 gene. The phenotype of resulting deletion strains was confirmed by plating on defined media with glucose as the only carbon source.

To eliminate the marker cassette, the deletion strains were grown on YPD/Et/Gly agar plates without selection for 1 day to allow for survival of cells that naturally excised the cassette by recombination of the 450-bp direct repeat formed between the endogenous PFK1 upstream region and the identical sequence introduced in the integration cassette. Subsequent plating of strains on YPD/Et/Gly agar containing 30 μM 5-fluoro-2′-deoxyuridine (FUDR) selected for the absence of the thymidine kinase gene. To identify marker-less PFK1 deletion strains, FUDR-resistant isolates were screened for reversion to nourseothricin sensitivity and loss of the marker cassette from the pfk1 locus was confirmed by PCR.

Oligonucleotide primer sequences are provided in the Table 4.

TABLE 3
PFK deletion cassette

| | | SEQ |
|---|---|---|
| | | ID |
| Region | Sequence | NO: |
| 47-bp sequence | CGCAAAACAGACGGACACTGAACCCCCCGCGCTTCAA | 1 |
| immediately | AACACCGACA | |
| upstream of PFK | | |
| Streptomyces | ATGACCACTCTGGATGACACCGCTTACCGATACCGAAC | 2 |
| noursei nat gene, | TTCCGTTCCTGGCGATGCCGAGGCTATTGAGGCTCTGG | |
| conferring | ATGGATCTTTCACCACTGACACCGTTTTCCGAGTGACC | |
| nourseothricin | GCTACTGGCGACGGCTTCACCCTGCGAGAGGTGCCTGT | |
| resistance codon | CGACCCTCCTCTCACCAAGGTTTTCCCTGACGATGAGT | |
| optimized for | CGGACGATGAGTCTGACGCTGGAGAGGACGGCGACCC | |
| expression in yeast | TGACTCTCGAACTTTCGTGGCTTACGGCGACGATGGAG | |
| | ACCTGGCCGGCTTTGTGGTCGTTTCTTACTCCGGATGG | |
| | AACCGACGACTGACCGTGGAGGACATCGAGGTCGCTC | |
| | CTGAGCACCGAGGTCATGGTGTCGGACGAGCTCTGATG | |
| | GGTCTCGCTACTGAGTTCGCTCGAGAGCGAGGTGCTGG | |
| | CCACCTGTGGCTCGAGGTCACCAACGTTAACGCCCCTG | |
| | CTATTCATGCCTACCGACGAATGGGTTTTACCCTGTGT | |
| | GGCCTCGATACTGCCCTGTACGACGGAACCGCTTCCGA | |
| | TGGAGAGCAGGCCCTCTACATGTCGATGCCCTGCCCTT | |
| | AA | |
| S. cerevisiae CYC1 | ACAGGCCCCTTTTCCTTTGTCGATATCATGTAATTAGTT | 3 |
| terminator | ATGTCACGCTTACATTCACGCCCTCCTCCCACATCCGCT | |
| | CTAACCGAAAAGGAAGGAGTTAGACAACCTGAAGTCT | |
| | AGGTCCCTATTTATTTTTTTTAATAGTTATGTTAGTATT | |
| | AAGAACGTTATTTATATTTCAAATTTTTCTTTTTTTTCT | |
| | GTACAAACGCGTGTACGCATGTAACATTATACTGAAAA | |
| | CCTTGCTTGAGAAGGTTTTGGGACGCTCGAAGGCTTTA | |
| | ATTTGC | |
| Y. lipolytica TEF1 | AGAGACCGGGTTGGCGGCGCATTTGTGTCCCAAAAAA | 4 |
| promoter | CAGCCCCAATTGCCCCAATTGACCCCAAATTGACCCAG | |
| | TAGCGGGCCCAACCCCGGCGAGAGCCCCCTTCTCCCCA | |
| | CATATCAAACCTCCCCCGGTTCCCACACTTGCCGTTAA | |
| | GGGCGTAGGGTACTGCAGTCTGGAATCTACGCTTGTTC | |
| | AGACTTTGTACTAGTTTCTTTGTCTGGCCATCCGGGTAA | |
| | CCCATGCCGGACGCAAAATAGACTACTGAAAATTTTTT | |
| | TGCTTTGTGGTTGGGACTTTAGCCAAGGGTATAAAAGA | |
| | CCACCGTCCCCGAATTACCTTTCCTCTTCTTTTCTCTCT | |
| | CTCCTTGTCAACTCACACCCGAAATCGTTAAGCATTTC | |
| | CTTCTGAGTATAAGAATCATTCAAA | |
| Herpes Simplex | ATGGCTTCTTACCCTTGCCACCAGCACGCTTCCGCTTTT | 5 |
| Virus thymidine | GACCAGGCCGCCCGATCCCGAGGACACTCCAACCGAC | |
| kinase gene | GAACCGCTCTGCGACCCCGACGACAGCAGGAGGCTAC | |
| hsvTDK codon- | CGAGGTTCGACTGGAGCAGAAGATGCCTACTCTGCTCC | |
| optimized for | GAGTGTACATCGACGGACCCCACGGTATGGGCAAGAC | |
| expression in Y. | CACTACCACTCAGCTGCTCGTCGCCCTGGGTTCGCGAG | |
| lipolytica | ATGACATTGTTTACGTGCCTGAGCCCATGACCTACTGG | |
| | CAGGTTCTCGGAGCTTCTGAGACTATCGCCAACATCTA | |
| | CACCACTCAGCATCGACTGGACCAGGGAGAGATCTCC | |
| | GCTGGAGATGCCGCTGTGGTCATGACCTCGGCCCAGAT | |
| | TACTATGGGCATGCCTTACGCTGTCACCGACGCTGTTC | |
| | TGGCTCCTCACATCGGTGGAGAGGCTGGATCTTCCCAT | |
| | GCTCCTCCTCCTGCTCTGACCCTCATCTTCGATCGACAC | |
| | CCTATTGCCGCTCTGCTCTGTTACCCCGCCGCTCGATAC | |
| | CTGATGGGCTCTATGACCCCTCAGGCCGTGCTGGCTTT | |
| | TGTCGCCCTCATCCCTCCCACCCTGCCTGGTACTAACAT | |
| | TGTGCTGGGTGCTCTCCCTGAGGACCGACATATCGATC | |
| | GACTCGCTAAGCGACAGCGACCTGGAGAGCGACTGGA | |
| | CCTCGCTATGCTGGCCGCTATTCGACGAGTGTACGGCC | |
| | TGCTCGCTAACACCGTCCGATACCTCCAGGGTGGTGGA | |
| | TCGTGGCGAGAGGACTGGGGACAGCTGTCTGGTACCG | |
| | CTGTGCCTCCTCAGGGTGCTGAGCCTCAGTCCAACGCT | |
| | GGACCTCGACCCCACATCGGTGACACCCTGTTCACTCT | |
| | CTTTCGAGCTCCTGAGCTGCTCGCTCCTAACGGCGACC | |
| | TGTACAACGTCTTCGCCTGGGCTCTGGATGTTCTCGCC | |
| | AAGCGACTCCGACCTATGCACGTCTTTATTCTGGACTA | |
| | CGATCAGTCGCCCGCTGGATGTCGAGATGCCCTGCTCC | |
| | AGCTCACCTCTGGCATGGTTCAGACTCATGTGACCACT | |
| | CCTGGATCCATCCCCACCATTTGCGATCTGGCTCGAAC | |
| | TTTCGCCCGAGAGATGGGAGAGGCCAACTAA | |
| Y. lipolytica TEF1 | GCTGCTTGTACCTAGTGCAACCCCAGTTTGTTAAAAAT | 6 |
| terminator | TAGTAGTCAAAAACTTCTGAGTTAGAAATTTGTGAGTG | |
| | TAGTGAGATTGTAGAGTATCATGTGTGTCCGTAAGTGA | |
| | AGTGTTATTGACTCTTAGTTAGTTTATCTAGTACTCGTT | |
| | TAGTTGACACTGATCTAGTATTTTACGAGGCGTATGAC | |
| | TTTAGCCAAGTGTTGTACTTAGTCTTCTCTCCAAACATG | |
| | AGAGGGCTCTGTCACTCAGTCGGCCTATGGGTGAGATG | |
| | GCTTGGTGAGATCTTTCGATAGTCTCGTCAAGATGGTA | |
| | GGATGATGGGGGAATACATTACTGCTCTCGTCAAGGA | |
| | AACCACAATCAGATCACACCATCCTCCATGGTATCCGA | |
| | TGACTCTCTTCTCCACAGT | |
| 450-bp sequence | CTTTTGATCCGATGGTTACTTTTTATGTTCTATTTTACA | 7 |
| corresponding to | TTAGCGTGGAAATAGACCATGCCATCTTTGGCACCCCG | |
| nucleotides −636 to | GAAAAACTTGATCCAATAGAGTTGTTGGGTGGAGCTA | |
| −187 upstream of | GTGACTGGCGGCAATTGGAGAGCTTCTAGAAGACGAA | |
| PFK | CCAGGAGCCCGATAGGAACTCCGTTGGCGTGAGTCGG | |
| | CCCCCCAGTAGCAATTCGAATCACGTGACGTGGAGTTT | |
| | TCCCTCGCCCGCGTTCCTGGATTGTCCCGGTGTGACGA | |
| | GGCCGACTGGATTTGATCACCCAACCCCACACGACGCA | |
| | TAATGTAAATGTATCATCATACAGTACATGCCCGAGTC | |
| | TAATGATTGGCTGGTTCACGGGGACCGGGAGCGTCCA | |
| | GTGGGCCGTATGGCGGTGGTTCAAACACCTCAGATCAG | |
| | CTCACTATCGGCTGAGACAATCCTTAACTGTTGGT | |
| 621-bp sequence | GGGGTTTGGTGTTGGAAATTAGGATATCTATTTGATTA | 8 |
| immediately | ATGTAGCTTGGTTTTGGACAAGAATGCTGATTGATACA | |
| downstream of | TCCGGTATCACTTGTATACAACGTAGGGGGCGGTGTAT | |
| PFK | GGGTGAAAATTTCGAAAGACTGAGAATCGAACTGGGA | |
| | GGTTTTTAAACTCCTGAAACTGTAGTTGGTGAACGAGC | |
| | CGAGGAGTATTTTAGACCCGAAACATTTGGTGCAACCA | |
| | GATACGCCAGGTAAACAATCTACACTACACTACATGG | |
| | GACGAGGGTTGAAAAGGTGGTGACAGGGATATTGCAG | |
| | ATGGGATTCTCGTGATGATGAGCCGTCAGCGTCTCGGC | |
| | TTGTCTTTGTGTTCAACGGCTTGTCTACGTCTGCTCGTA | |
| | TCCACCTGTCATGTTGGTTGTTCCATGCTTATAGGCCTG | |
| | TCTGTTGGGGGCAGTAAGGTTTTGAGAAGTGTGTAGTA | |
| | TGGGGATGTATTATCTTGGGGTTTTGGTTGAGATATTA | |
| | GTAATCTGAGAGTTTGAGGGTATTTCTGGCGGTTTGTG | |
| | TATATAAGTCCGGCTTTTTCCAAGTCGATATCTCCTTTT | |
| | GTTGTTGTTTTGGGGCCGCTTTTATTTCTTCCAGGGCTG | |
| | TAGGCGAAATTC | |

TABLE 4
Oligonucleotide primers used to amplify PFK deletion cassette in two
overlapping fragments

| | | | SEQ |
|---|---|---|---|
| Primer | Description | Sequence | ID NO: |
| NP2782 | 5′ fragment forward primer | CGCAAAACAGACGGACACTGAA | 9 |
| | | CCCCCCGCGCTTCAAAACACCGA | |
| | | CAATGACCACTCTGGATGACACC | |
| NP356 | 5′ fragment reverse primer | TCTCCATCGGAAGCGGTTCCGTC | 10 |
| NP355 | 3′ fragment forward primer | AACTTCCGTTCCTGGCGATGCCG | 11 |
| NP3531 | 3′ fragment reverse primer | GAATTTCGCCTACAGCCCTGG | 12 |

### Growth and Lipid Assays

To evaluate growth on glucose, strains were patched on YNB plates (6.7 g/L Yeast Nitrogen Base without amino acids, 20 g/L agar) or cultured in lipid production media (0.5 g/L urea, 1.5 g/L yeast extract, 0.85 g/L casamino acids, 1.7 g/L Yeast Nitrogen Base without amino acids and ammonium sulfate, 100 g/L glucose, and 5.11 g/L potassium hydrogen phthalate) [10]. Growth in lipid production media was tested by growing strains overnight in YPD, washing with sterile water, and inoculating into the lipid production media at a starting OD₆₀₀ (Optical Density measured at 600 nm) of 0.05. OD₆₀₀ measurements to monitor growth were taken after culturing for 2 days in shake flasks.

To measure lipid accumulation, strains were grown in glycerol (YPG) for 24 h and then switched to a modified Verduyn media (modified to contain no ammonium sulfate and 100 g/L glucose) to induce lipid production. The cells grown in YPG were pelleted, washed with water and resuspended in the modified Verduyn media [50] and cultured for seven days. These characterizations were carried out in 96-well, 48-well or 24-well deep well plates and 250-mL shake flasks. The lipid Bodipy assay described in previous work [10] was used with one modification: PBS was used instead of the master mix previously described. Lipid accumulation was measured as fluorescence units normalized to the OD₆₀₀ (FI/OD).

### Example 3—Engineering the Xpk/Pta Pathway into a Δpfk1 Strain

To assemble the Xpk/Pta/Δpfk1 pathway in Y. lipolytica, CaXPK(v1) and BsPTA(v1) were expressed in NS1047 (Δpfk1) (FIGS. 6A and 6B). With each transformation step, the best transformant was screened for using appropriate enzymatic assays and growth and lipid accumulation on glucose were measured (see Table 5). Noticeable Xpk and Pta activities were obtained in the course of constructing strain NS1352 (FIG. 6A). However, NS1352 containing three copies of CaXPK(v1) and two copies of BsPTA(v1), showed minimal improvement in growth or lipid accumulation on glucose when compared to NS1047 (FIG. 6B). One possibility for the lack of improvement was that Xpk and Pta activities were still too low for a functional pathway. As addition of multiple copies of CaXPK(v1) and BsPTA(v1) genes yielded only incremental improvements in enzymatic activities (FIG. 6A), the codon optimization strategies were revised to improve gene expression.

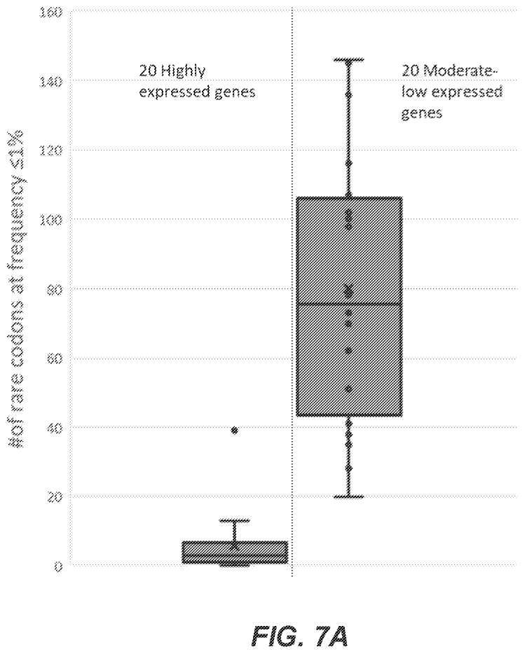

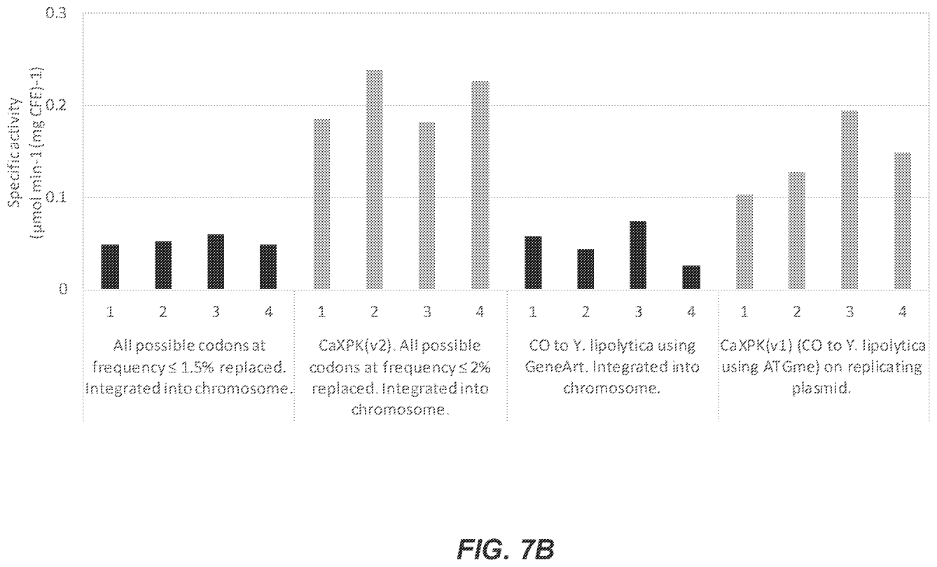

To further improve Xpk and Pta activity in Y. lipolytica, different codon optimization strategies were tested on the top performing genes BsPTA, TsPTA and CaXPK. TsPTA codon-optimized to Y. lipolytica (GeneArt) exhibited the highest activity and is referred to as TsPTA(v2) from hereon (See Table 9). Three additional versions of CaXPK were designed and tested (FIG. 7B). The highest Xpk activity was obtained when all codons present at a frequency≤2% were replaced with their higher frequency counterparts and this gene is referred to as CaXPK(v2) (see Table 9).

To determine whether TsPTA(v2) and CaXPK(v2) increase flux through the Xpk/Pta pathway, growth and lipid accumulation of NS1352 expressing these genes in the presence of glucose was evaluated. Addition of TsPTA(v2) quadrupled Pta activity (NS1420, FIG. 6A) but growth and lipid production on glucose remained unchanged (FIG. 6B), suggesting that Xpk activity could still be limiting. Consistent with this hypothesis, addition of CaXPK(v2) improved lipid accumulation to near YB-392 levels (NS1457, FIG. 6B, grey bars). Growth on glucose also improved but was still deficient compared to YB-392 (NS1457, FIG. 6B, black bars). Addition of a second copy of CaXPK(v2) almost quadrupled growth on glucose compared to its parent strain (NS1457 and NS1475, FIG. 6B, black bars). Lipid accumulation remained similar to the parent strain NS1457 and YB-392 (FIG. 6B, grey bars). The observation that lipid accumulation was restored with fewer copies of XPK than were required to restore wild-type growth to a Δpfk1 strain suggests that more Xpk activity is needed for growth than lipid production.

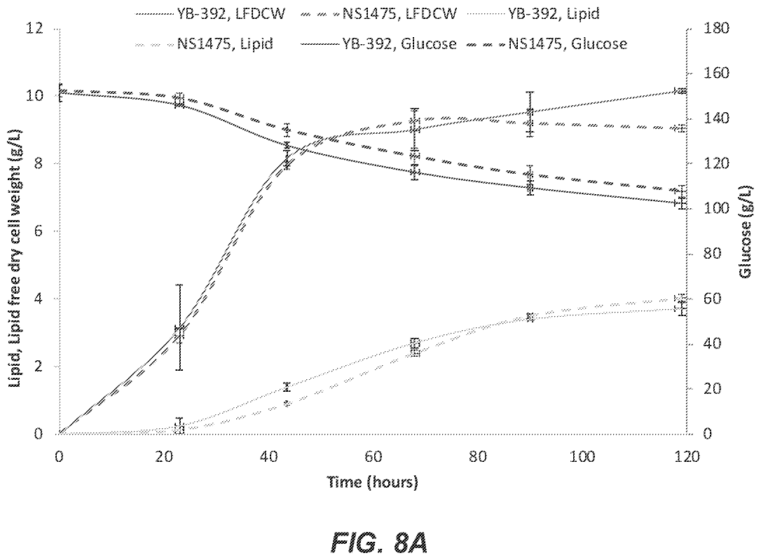

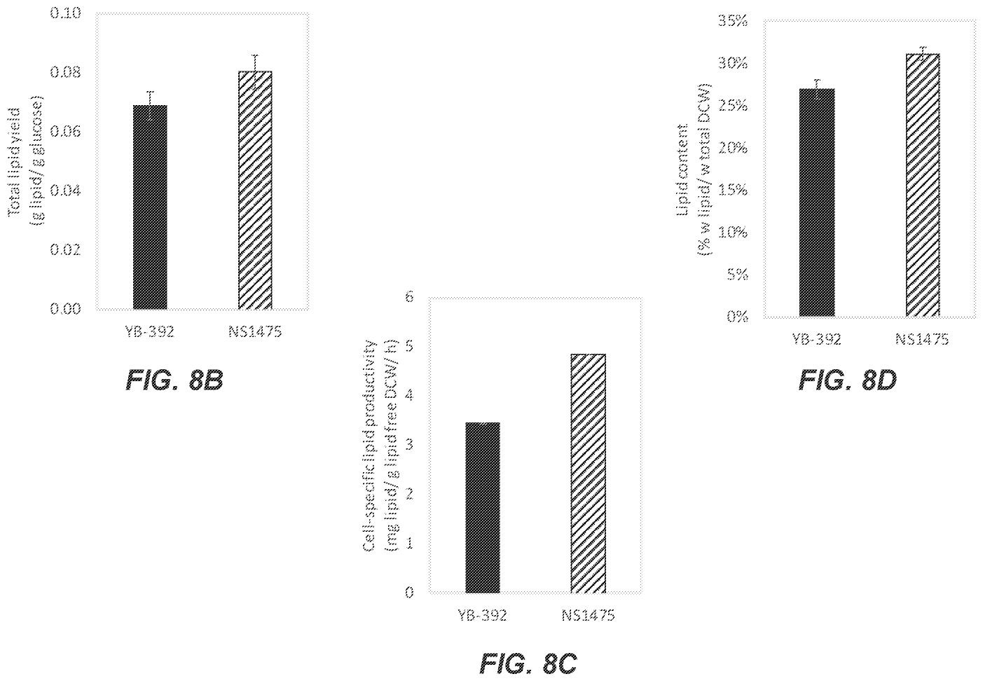

To further characterize the Xpk/Pta/Δpfk1 pathway strain NS1475, batch fermentations were carried out in 1-L bioreactors with YB-392 included as control. Two replicate fermentation experiments comparing YB-392 and NS1475 were conducted on two separate occasions to account for any culturing variations. NS1475 was comparable to the wild-type YB-392 in terms of growth and total lipid accumulated (FIG. 8A). NS1475 recorded an improved total lipid yield (+16%), cell-specific lipid productivity (+41%) and lipid content (+16%) over YB-392 (FIGS. 8B-8D). These results confirm that the Xpk/Pta pathway can rescue growth and lipid defects of a PFK deletion while also improving bioprocess parameters like yield and cell-specific productivity.

TABLE 5
Steps involved in the construction of strain NS1475

| | | # of | |
|---|---|---|---|
| | | isolates | |
| Strain | Genotype | screened | Screening assay |
| NS1047 | Δpfk1 | 7 | Colony PCR followed |
| | | | by growth on YNB plates |
| NS1281 | Δpfk1, CaXPK(v1) | 10 | Ferric hydroxamate assay |
| NS1292 | Δpfk1, 2xCaXPK(v1) | 10 | Ferric hydroxamate assay |
| NS1322 | Δpfk1, 3xCaXPK(v1) | 10 | Ferric hydroxamate assay |
| NS1341 | Δpfk1, 3xCaXPK(v1), | 10 | DTNB assay |
| | BsPTA(v1) | | |
| NS1352 | Δpfk1, 3xCaXPK(v1), | 8 | DTNB assay |
| | 2xBsPTA(v1) | | |
| NS1420 | Δpfk1, 3xCaXPK(v1), | 10 | DTNB assay |
| | 2xBsPTA(v1), | | |
| | TsPTA(v2) | | |
| NS1457 | Δpfk1, 3xCaXPK(v1), | 46 | Growth and lipid assay on |
| | 2xBsPTA(v1), | | lipid production media, |
| | TsPTA(v2), | | microscopy |
| | CaXPK(v2) | | |
| NS1475 | Δpfk1, 3xCaXPK(v1), | 90 | Growth and lipid assay on |
| | 2xBsPTA(v1), | | lipid production media, |
| | TsPTA(v2), | | microscopy |
| | 2xCaXPK(v2) | | |

TABLE 6
pta expression cassette used for finding functional pta in Y. lipolytica

| | | SEQ |
|---|---|---|
| | | ID |
| Region | Sequence | NO: |
| S. cerevisiae FBA1 | GATCCAACTGGCACCGCTGGCTTGAACAACAATACC | 13 |
| promoter | AGCCTTCCAACTTCTGTAAATAACGGCGGTACGCCA | |
| | GTGCCACCAGTACCGTTACCTTTCGGTATACCTCCTT | |
| | TCCCCATGTTTCCAATGCCCTTCATGCCTCCAACGGC | |
| | TACTATCACAAATCCTCATCAAGCTGACGCAAGCCC | |
| | TAAGAAATGAATAACAATACTGACAGTACTAAATAA | |
| | TTGCCTACTTGGCTTCACATACGTTGCATACGTCGAT | |
| | ATAGATAATAATGATAATGACAGCAGGATTATCGTA | |
| | ATACGTAATAGTTGAAAATCTCAAAAATGTGTGGGT | |
| | CATTACGTAAATAATGATAGGAATGGGATTCTTCTA | |
| | TTTTTCCTTTTTCCATTCTAGCAGCCGTCGGGAAAAC | |
| | GTGGCATCCTCTCTTTCGGGCTCAATTGGAGTCACGC | |
| | TGCCGTGAGCATCCTCTCTTTCCATATCTAACAACTG | |
| | AGCACGTAACCAATGGAAAAGCATGAGCTTAGCGTT | |
| | GCTCCAAAAAAGTATTGGATGGTTAATACCATTTGT | |
| | CTGTTCTCTTCTGACTTTGACTCCTCAAAAAAAAAAA | |
| | ATCTACAATCAACAGATCGCTTCAATTACGCCCTCA | |
| | CAAAAACTTTTTTCCTTCTTCTTCGCCCACGTTAAAT | |
| | TTTATCCCTCATGTTGTCTAACGGATTTCTGCACTTG | |
| | ATTTATTATAAAAAGACAAAGACATAATACTTCTCT | |
| | ATCAATTTCAGTTATTGTTCTTCCTTGCGTTATTCTTC | |
| | TGTTCTTCTTTTTCTTTTGTCATATATAACCATAACCA | |
| | AGTAATACATATTCAAA | |
| E. coli hph gene, | ATGAAGAAGCCCGAGCTGACCGCTACCTCTGTTGAG | 14 |
| conferring | AAGTTCCTGATTGAGAAGTTTGATTCCGTTTCCGACC | |
| hygromycin B | TGATGCAGCTGTCCGAGGGCGAGGAGTCTCGAGCCT | |
| resistance | TCTCCTTTGACGTGGGCGGACGAGGTTACGTTCTGC | |
| (GenBank: | GAGTGAACTCGTGTGCCGACGGCTTCTACAAGGATC | |
| AEJ60084.1) | GATACGTCTACCGACACTTTGCTTCTGCCGCTCTGCC | |
| codon-optimized | CATCCCTGAGGTTCTCGACATTGGCGAGTTCTCTGAG | |
| for expression in | TCCCTCACCTACTGCATCTCTCGACGAGCTCAGGGA | |
| Y. lipolytica | GTCACCCTGCAGGACCTCCCTGAGACTGAGCTGCCT | |
| (Genscript) | GCTGTCCTCCAGCCTGTTGCTGAGGCCATGGACGCT | |
| | ATCGCTGCTGCTGATCTGTCCCAGACCTCGGGTTTCG | |
| | GCCCCTTTGGACCTCAGGGAATTGGACAGTACACCA | |
| | CTTGGCGAGACTTCATCTGTGCTATTGCCGATCCTCA | |
| | CGTCTACCATTGGCAGACCGTTATGGACGATACTGT | |
| | GTCGGCTTCTGTCGCTCAGGCTCTGGACGAGCTGAT | |
| | GCTCTGGGCCGAGGATTGCCCCGAGGTTCGACACCT | |
| | GGTGCATGCTGACTTCGGTTCCAACAACGTTCTCACC | |
| | GACAACGGCCGAATCACTGCCGTGATTGACTGGTCC | |
| | GAGGCTATGTTTGGCGACTCGCAGTACGAGGTGGCC | |
| | AACATCTTCTTTTGGCGACCCTGGCTGGCTTGTATGG | |
| | AGCAGCAGACCCGATACTTCGAGCGACGACATCCTG | |
| | AGCTCGCTGGATCCCCTCGACTGCGAGCTTACATGC | |
| | TCCGAATTGGTCTGGACCAGCTCTACCAGTCGCTGG | |
| | TGGATGGCAACTTTGACGATGCTGCCTGGGCTCAGG | |
| | GACGATGTGACGCCATCGTGCGATCTGGCGCTGGAA | |
| | CCGTCGGACGAACTCAGATTGCCCGACGATCCGCTG | |
| | CTGTCTGGACCGACGGATGCGTGGAGGTCCTGGCTG | |
| | ATTCGGGTAACCGACGACCCTCTACTCGACCTCGAG | |
| | CTAAGGAGTAA | |
| S. cerevisiae FBA1 | GTTAATTCAAATTAATTGATATAGTTTTTTAATGAGT | 15 |
| terminator | ATTGAATCTGTTTAGAAATAATGGAATATTATTTTTA | |
| | TTTATTTATTTATATTATTGGTCGGCTCTTTTCTTCTG | |
| | AAGGTCAATGACAAAATGATATGAAGGAAATAATG | |
| | ATTTCTAAAATTTTACAACGTAAGATATTTTTACAAA | |
| | AGCCTAGCTCATCTTTTGTCA | |
| Y. lipolytica EXP1 | GAGTTTGGCGCCCGTTTTTTCGAGCCCCACACGTTTC | 16 |
| promoter | GGTGAGTATGAGCGGCGGCAGATTCGAGCGTTTCCG | |
| | GTTTCCGCGGCTGGACGAGAGCCCATGATGGGGGCT | |
| | CCCACCACCAGCAATCAGGGCCCTGATTACACACCC | |
| | ACCTGTAATGTCATGCTGTTCATCGTGGTTAATGCTG | |
| | CTGTGTGCTGTGTGTGTGTGTTGTTTGGCGCTCATTG | |
| | TTGCGTTATGCAGCGTACACCACAATATTGGAAGCT | |
| | TATTAGCCTTTCTATTTTTTCGTTTGCAAGGCTTAAC | |
| | AACATTGCTGTGGAGAGGGATGGGGATATGGAGGC | |
| | CGCTGGAGGGAGTCGGAGAGGCGTTTTGGAGCGGCT | |
| | TGGCCTGGCGCCCAGCTCGCGAAACGCACCTAGGAC | |
| | CCTTTGGCACGCCGAAATGTGCCACTTTTCAGTCTAG | |
| | TAACGCCTTACCTACGTCATTCCATGCATGCATGTTT | |
| | GCGCCTTTTTTCCCTTGCCCTTGATCGCCACACAGTA | |
| | CAGTGCACTGTACAGTGGAGGTTTTGGGGGGGTCTT | |
| | AGATGGGAGCTAAAAGCGGCCTAGCGGTACACTAGT | |
| | GGGATTGTATGGAGTGGCATGGAGCCTAGGTGGAGC | |
| | CTGACAGGACGCACGACCGGCTAGCCCGTGACAGAC | |
| | GATGGGTGGCTCCTGTTGTCCACCGCGTACAAATGT | |
| | TTGGGCCAAAGTCTTGTCAGCCTTGCTTGCGAACCTA | |
| | ATTCCCAATTTTGTCACTTCGCACCCCCATTGATCGA | |
| | GCCCTAACCCCTGCCCATCAGGCAATCCAATTAAGC | |
| | TCGCATTGTCTGCCTTGTTTAGTTTGGCTCCTGCCCG | |
| | TTTCGGCGTCCACTTGCACAAACACAAACAAGCATT | |
| | ATATATAAGGCTCGTCTCTCCCTCCCAACCACACTCA | |
| | CTTTTTTGCCCGTCTTCCCTTGCTAACACAAAAGTCA | |
| | AGAACACAAACAACCACCCCAACCCCCTTACACACA | |
| | AGACATATCTACAGCA | |
| pta genes | [gene_of_interest] | N/A |
| Y. lipolytica CYC1 | GCGTCTACAACTGGACCCTTAGCCTGTATATATCAAT | 17 |
| terminator | TGATTATTTAAAGATTTGGTCGGTAGGCGGTTCGTAT | |
| | TGTACAATGGGATCTGTTACTGAGGTGGATCTACCC | |
| | AACTTGCGAGATTCAATTGCGAGATTCAATCGCGAG | |
| | ATTCAATTGCGAGAATCAGTTGCGAGTTGTTCTAAC | |
| | ACTCAGCTTCTACGAGCGCTTGTATTAGGACGAGTG | |
| | ATACTCCGTGGGGCGACGGCTTCTCTTGCGTCTTCTG | |
| | TTGTATTCTTTCTTACACTATCGTCCATCTCCAACCA | |
| | CCTCGTAC | |

TABLE 7
Replicating plasmid containing the xpk expression cassette used for
finding functional xpk in Y. lipolytica

| | | SEQ |
|---|---|---|
| Region | Sequence | ID NO: |
| Autonomous | AGCTAGCTCGTCGTGTTCAGGAACTGTTCGATGGTTCG | 18 |
| replicating | GAGAGAGTCGCCGCCCAGAACATACGCGCACCGATGT | |
| sequence | CAGCAGACAGCCTTATTACAAGTACAGTATGTACATAC | |
| ARS68 | TACTGTATATTCAAGCAAGTATATCCGTAGGGTGCGGG | |
| | TGATTTGGATCTAAGGTTCGTACTCAACACTCACGAGC | |
| | AGCTTGCCTATGTTACATCCTTTTATCAGACATAACAT | |
| | AATTGGAGTTTACTTACACACGGGGTGTACCTGTATGA | |
| | GCACCACCTACAATTGTAGCACTGGTACTTGTACAAAG | |
| | AATTTATTCGTACGAATCACAGGAACGGCCGCCCTCAC | |
| | CGAACCAGCGAATACCTCAGCGGTCCCCTGCAGTGACT | |
| | CAACAAAGCGATATGAACATCTTGCGATGGTATCCTGC | |
| | TGATAGTTTTTACTGTACAAACACCTGTGTAGCTCCTTC | |
| | TAGCATTTTTAAGTTATTCACACCTCAAGGGGAGGGAT | |
| | AAATTAAATAAATTCCAAAAGCGAAGATCGAGAAACT | |
| | AAATTAAAATTCCAAAAACGAAGTTGGAACACAACCC | |
| | CCCGAAAAAAAACAACAAGCAAAAAACCCAACAAAAT | |
| | AAACAAAAACAAAATAAATATATAACAAAATAAATAT | |
| | ATAACTACCAGTATCTGACTAAAAGTTCAAATACTCGT | |
| | ACTTACAACAAATAGAAATGAGCCGGCCAAAATTCTG | |
| | CAGAAAAAATTTTCAAACAAGTACTTTCAAACAAGTAC | |
| | TGGTATAATTAAATTAAAAAACACATCAAAGTATCATA | |
| | ACGTTAGTTATTTTATTTTATTTAATAAAAGAAAACAA | |
| | CAAAATGGGCTCAAAACTTTCAACTTATACG | |
| Centromere | ATACATACCAAATAACAATTTAGTATTTATCTAAGTGC | 19 |
| CEN1-1 | TTTTCGTAGATAATGGAATACAAATGGATATCCAGAGT | |
| | ATACACATGGATAGTATACACTGACACGACAATTCTGT | |
| | ATCTCTTTATGTTAACTACTGTGAGGCGTTAAATAGAG | |
| | CTTGATATATAAAATGTTACATTTCACAGTCTGAACTTT | |
| | TGCAGATTACCTAATTTGGTAAGATATTAATTATGAAC | |
| | TGAAAGTTGATG | |
| Arxula | TGCGTCGGAACGGGATATGCATTCCCCTAGTTTCGCCG | 20 |
| adeninivorans | CAGTGCAGAATCAGGCGGTTTCTTTGCACCACACCACA | |
| ADHI promoter | TACGGAGGATGACGGGCATTATTGATGTTGAATAGTAA | |
| | CCTGATCGTGACTAGTATGACGGAACCCAACAGCAAC | |
| | AGCCGACCGTTTGTGAGCGTTTTTGCGGCCGGTCAGGC | |
| | GAGTTTTTCCGGCCTGCCAATGGTCCTTCCGTACCCTTT | |
| | ACCCTGTACGCTGTACCTGCCACGGATAGGCCGTGCTC | |
| | CACCTGCTCACTATGGTGGGTGCGGGGAAAACAACAG | |
| | GCAGGCTCAATTGCTCTGCAAATGGGTTGAGGGGGTG | |
| | ATTGATGTCACTGGTACACCAACAGGGGAATGCTCGGC | |
| | GTTGATTTTGGGCCACCTCTTTTGTTTGCCAGAGCTTGT | |
| | CTCTATTGTCAAATTTAACGGTCTGCAACTGTTGCCCA | |
| | AAATGGGACAATGATCCGATGCCTGCATAGACACCCT | |
| | GCTTGAGGGTGCGATCGCCCTAATACGAGGCAAACCA | |
| | AGTTTTCCAATTGACCTTCAATTGACGAGCGGTTGTTG | |
| | CGACAGGGGACTGGAGTGCTACCTGTTTAGAGTTCAAA | |
| | TCCGTCACCCAGCATTGAAAGTTTTTCCCCGCATTGGA | |
| | TGATTGCAATGCCGCTAACCCGCTCATCCGCCAAAGTT | |
| | CATAGTCCCACCCTGCCTCGACTTATCGGACCACATGG | |
| | GGCTCCCTTATGCGCGCGCATATGGCGCTTGATTGCTT | |
| | TTTGGTCAACGTTTGGGACAAATTTCCTTTGTTAAGGC | |
| | GGACCCGCCAGCAGATACGAAGGTATAAATAGGGCTC | |
| | ACTTTCACCATCTTGTCCATTCAATTGCAAGACTCAAA | |
| | AGTAATA | |
| Streptomyces | ATGACCACTCTGGATGACACCGCTTACCGATACCGAAC | 21 |
| noursei Nat1 | TTCCGTTCCTGGCGATGCCGAGGCTATTGAGGCTCTGG | |
| gene, conferring | ATGGATCTTTCACCACTGACACCGTTTTCCGAGTGACC | |
| Nourseothricin | GCTACTGGCGACGGCTTCACCCTGCGAGAGGTGCCTGT | |
| resistance | CGACCCTCCTCTCACCAAGGTTTTCCCTGACGATGAGT | |
| (GenBank: | CGGACGATGAGTCTGACGCTGGAGAGGACGGCGACCC | |
| CAA51674.1) | TGACTCTCGAACTTTCGTGGCTTACGGCGACGATGGAG | |
| codon- | ACCTGGCCGGCTTTGTGGTCGTTTCTTACTCCGGATGG | |
| optimized for | AACCGACGACTGACCGTGGAGGACATCGAGGTCGCTC | |
| expression in Y. | CTGAGCACCGAGGTCATGGTGTCGGACGAGCTCTGATG | |
| lipolytica | GGTCTCGCTACTGAGTTCGCTCGAGAGCGAGGTGCTGG | |
| (Genscript) | CCACCTGTGGCTCGAGGTCACCAACGTTAACGCCCCTG | |
| | CTATTCATGCCTACCGACGAATGGGTTTTACCCTGTGT | |
| | GGCCTCGATACTGCCCTGTACGACGGAACCGCTTCCGA | |
| | TGGAGAGCAGGCCCTCTACATGTCGATGCCCTGCCCTT | |
| | AA | |
| S. cerevisiae | ACAGGCCCCTTTTCCTTTGTCGATATCATGTAATTAGTT | 3 |
| CYC1 | ATGTCACGCTTACATTCACGCCCTCCTCCCACATCCGCT | |
| terminator | CTAACCGAAAAGGAAGGAGTTAGACAACCTGAAGTCT | |
| | AGGTCCCTATTTATTTTTTTTAATAGTTATGTTAGTATT | |
| | AAGAACGTTATTTATATTTCAAATTTTTCTTTTTTTTCT | |
| | GTACAAACGCGTGTACGCATGTAACATTATACTGAAAA | |
| | CCTTGCTTGAGAAGGTTTTGGGACGCTCGAAGGCTTTA | |
| | ATTTGC | |
| Y. lipolytica | AGAGACCGGGTTGGCGGCGCATTTGTGTCCCAAAAAA | 4 |
| TEF1 promoter | CAGCCCCAATTGCCCCAATTGACCCCAAATTGACCCAG | |
| | TAGCGGGCCCAACCCCGGCGAGAGCCCCCTTCTCCCCA | |
| | CATATCAAACCTCCCCCGGTTCCCACACTTGCCGTTAA | |
| | GGGCGTAGGGTACTGCAGTCTGGAATCTACGCTTGTTC | |
| | AGACTTTGTACTAGTTTCTTTGTCTGGCCATCCGGGTAA | |
| | CCCATGCCGGACGCAAAATAGACTACTGAAAATTTTTT | |
| | TGCTTTGTGGTTGGGACTTTAGCCAAGGGTATAAAAGA | |
| | CCACCGTCCCCGAATTACCTTTCCTCTTCTTTTCTCTCT | |
| | CTCCTTGTCAACTCACACCCGAAATCGTTAAGCATTTC | |
| | CTTCTGAGTATAAGAATCATTCAAA | |
| xpk gene of | [gene_of_interest] | N/A |
| interest | | |
| A. | GCGGTTTAGATTTTCCAATTGTAAATATATTACTGTACC | 22 |
| adeninivorans | ATTCTGTACTAAATAACGTGTTTTTTATACTACTTCCTA | |
| CYC1 | TCTATATTCTATATCGTTACTGGCATATATATATCGTTG | |
| terminator | CTGGAGGTCGAAGGTGAAATTTCACTTGCCTTTCTCTT | |
| | CCCCGAGCCGCACGCCGCCATATCGTTATCTTGATTGA | |
| | GCCCGACATCATGATCACTAACTGATTATGCTCCAGCA | |
| | GGAGCAGGTTGTAGAGCTCCAAGATTTGCGAGGAGGT | |
| | GGAGAGAATAGCACGGGACAGAGAGTACGATGG | |

TABLE 8
pta or xpk expression cassette used in the construction of strains NS1475
and NS1656-57

| | | SEQ |
|---|---|---|
| Region | Sequence | ID NO: |
| Y. lipolytica | AGAGACCGGGTTGGCGGCGCATTTGTGTCCCAAAAAAC | 4 |
| TEF1 promoter | AGCCCCAATTGCCCCAATTGACCCCAAATTGACCCAGT | |
| | AGCGGGCCCAACCCCGGCGAGAGCCCCCTTCTCCCCAC | |
| | ATATCAAACCTCCCCCGGTTCCCACACTTGCCGTTAAGG | |
| | GCGTAGGGTACTGCAGTCTGGAATCTACGCTTGTTCAG | |
| | ACTTTGTACTAGTTTCTTTGTCTGGCCATCCGGGTAACC | |
| | CATGCCGGACGCAAAATAGACTACTGAAAATTTTTTTG | |
| | CTTTGTGGTTGGGACTTTAGCCAAGGGTATAAAAGACC | |
| | ACCGTCCCCGAATTACCTTTCCTCTTCTTTTCTCTCTCTC | |
| | CTTGTCAACTCACACCCGAAATCGTTAAGCATTTCCTTC | |
| | TGAGTATAAGAATCATTCAAA | |
| Herpes | ATGGCTTCTTACCCTTGCCACCAGCACGCTTCCGCTTTT | 5 |
| Simplex Virus | GACCAGGCCGCCCGATCCCGAGGACACTCCAACCGACG | |
| thymidine | AACCGCTCTGCGACCCCGACGACAGCAGGAGGCTACCG | |
| kinase gene | AGGTTCGACTGGAGCAGAAGATGCCTACTCTGCTCCGA | |
| hsvTDK | GTGTACATCGACGGACCCCACGGTATGGGCAAGACCAC | |
| codon- | TACCACTCAGCTGCTCGTCGCCCTGGGTTCGCGAGATGA | |
| optimized for | CATTGTTTACGTGCCTGAGCCCATGACCTACTGGCAGGT | |
| expression in | TCTCGGAGCTTCTGAGACTATCGCCAACATCTACACCAC | |
| Y. lipolytica | TCAGCATCGACTGGACCAGGGAGAGATCTCCGCTGGAG | |
| | ATGCCGCTGTGGTCATGACCTCGGCCCAGATTACTATGG | |
| | GCATGCCTTACGCTGTCACCGACGCTGTTCTGGCTCCTC | |
| | ACATCGGTGGAGAGGCTGGATCTTCCCATGCTCCTCCTC | |
| | CTGCTCTGACCCTCATCTTCGATCGACACCCTATTGCCG | |
| | CTCTGCTCTGTTACCCCGCCGCTCGATACCTGATGGGCT | |
| | CTATGACCCCTCAGGCCGTGCTGGCTTTTGTCGCCCTCA | |
| | TCCCTCCCACCCTGCCTGGTACTAACATTGTGCTGGGTG | |
| | CTCTCCCTGAGGACCGACATATCGATCGACTCGCTAAG | |
| | CGACAGCGACCTGGAGAGCGACTGGACCTCGCTATGCT | |
| | GGCCGCTATTCGACGAGTGTACGGCCTGCTCGCTAACA | |
| | CCGTCCGATACCTCCAGGGTGGTGGATCGTGGCGAGAG | |
| | GACTGGGGACAGCTGTCTGGTACCGCTGTGCCTCCTCA | |
| | GGGTGCTGAGCCTCAGTCCAACGCTGGACCTCGACCCC | |
| | ACATCGGTGACACCCTGTTCACTCTCTTTCGAGCTCCTG | |
| | AGCTGCTCGCTCCTAACGGCGACCTGTACAACGTCTTCG | |
| | CCTGGGCTCTGGATGTTCTCGCCAAGCGACTCCGACCTA | |
| | TGCACGTCTTTATTCTGGACTACGATCAGTCGCCCGCTG | |
| | GATGTCGAGATGCCCTGCTCCAGCTCACCTCTGGCATGG | |
| | TTCAGACTCATGTGACCACTCCTGGATCCATCCCCACCA | |
| | TTTGCGATCTGGCTCGAACTTTCGCCCGAGAGATGGGA | |
| | GAGGCCAACTAA | |
| S. cerevisiae | GTTAATTCAAATTAATTGATATAGTTTTTTAATGAGTAT | |
| FBA1 | TGAATCTGTTTAGAAATAATGGAATATTATTTTTATTTA | |
| terminator | TTTATTTATATTATTGGTCGGCTCTTTTCTTCTGAAGGTC | |
| | AATGACAAAATGATATGAAGGAAATAATGATTTCTAAA | |
| | ATTTTACAACGTAAGATATTTTTACAAAAGCCTAGCTCA | |
| | TCTTTTGTCA | |
| Arxula | TGCGTCGGAACGGGATATGCATTCCCCTAGTTTCGCCGC | 20 |
| adeninivorans | AGTGCAGAATCAGGCGGTTTCTTTGCACCACACCACAT | |
| ADH1 | ACGGAGGATGACGGGCATTATTGATGTTGAATAGTAAC | |
| promoter | CTGATCGTGACTAGTATGACGGAACCCAACAGCAACAG | |
| | CCGACCGTTTGTGAGCGTTTTTGCGGCCGGTCAGGCGA | |
| | GTTTTTCCGGCCTGCCAATGGTCCTTCCGTACCCTTTAC | |
| | CCTGTACGCTGTACCTGCCACGGATAGGCCGTGCTCCAC | |
| | CTGCTCACTATGGTGGGTGCGGGGAAAACAACAGGCAG | |
| | GCTCAATTGCTCTGCAAATGGGTTGAGGGGGTGATTGA | |
| | TGTCACTGGTACACCAACAGGGGAATGCTCGGCGTTGA | |
| | TTTTGGGCCACCTCTTTTGTTTGCCAGAGCTTGTCTCTAT | |
| | TGTCAAATTTAACGGTCTGCAACTGTTGCCCAAAATGG | |
| | GACAATGATCCGATGCCTGCATAGACACCCTGCTTGAG | |
| | GGTGCGATCGCCCTAATACGAGGCAAACCAAGTTTTCC | |
| | AATTGACCTTCAATTGACGAGCGGTTGTTGCGACAGGG | |
| | GACTGGAGTGCTACCTGTTTAGAGTTCAAATCCGTCACC | |
| | CAGCATTGAAAGTTTTTCCCCGCATTGGATGATTGCAAT | |
| | GCCGCTAACCCGCTCATCCGCCAAAGTTCATAGTCCCAC | |
| | CCTGCCTCGACTTATCGGACCACATGGGGCTCCCTTATG | |
| | CGCGCGCATATGGCGCTTGATTGCTTTTTGGTCAACGTT | |
| | TGGGACAAATTTCCTTTGTTAAGGCGGACCCGCCAGCA | |
| | GATACGAAGGTATAAATAGGGCTCACTTTCACCATCTT | |
| | GTCCATTCAATTGCAAGACTCAAAAGTAATA | |
| E. coli hph | ATGAAGAAGCCCGAGCTGACCGCTACCTCTGTTGAGAA | 14 |
| gene, | GTTCCTGATTGAGAAGTTTGATTCCGTTTCCGACCTGAT | |
| conferring | GCAGCTGTCCGAGGGCGAGGAGTCTCGAGCCTTCTCCT | |
| hygromycin B | TTGACGTGGGCGGACGAGGTTACGTTCTGCGAGTGAAC | |
| resistance | TCGTGTGCCGACGGCTTCTACAAGGATCGATACGTCTAC | |
| codon- | CGACACTTTGCTTCTGCCGCTCTGCCCATCCCTGAGGTT | |
| optimized for | CTCGACATTGGCGAGTTCTCTGAGTCCCTCACCTACTGC | |
| expression in | ATCTCTCGACGAGCTCAGGGAGTCACCCTGCAGGACCT | |
| Y. lipolytica | CCCTGAGACTGAGCTGCCTGCTGTCCTCCAGCCTGTTGC | |
| | TGAGGCCATGGACGCTATCGCTGCTGCTGATCTGTCCCA | |
| | GACCTCGGGTTTCGGCCCCTTTGGACCTCAGGGAATTGG | |
| | ACAGTACACCACTTGGCGAGACTTCATCTGTGCTATTGC | |
| | CGATCCTCACGTCTACCATTGGCAGACCGTTATGGACG | |
| | ATACTGTGTCGGCTTCTGTCGCTCAGGCTCTGGACGAGC | |
| | TGATGCTCTGGGCCGAGGATTGCCCCGAGGTTCGACAC | |
| | CTGGTGCATGCTGACTTCGGTTCCAACAACGTTCTCACC | |
| | GACAACGGCCGAATCACTGCCGTGATTGACTGGTCCGA | |
| | GGCTATGTTTGGCGACTCGCAGTACGAGGTGGCCAACA | |
| | TCTTCTTTTGGCGACCCTGGCTGGCTTGTATGGAGCAGC | |
| | AGACCCGATACTTCGAGCGACGACATCCTGAGCTCGCT | |
| | GGATCCCCTCGACTGCGAGCTTACATGCTCCGAATTGGT | |
| | CTGGACCAGCTCTACCAGTCGCTGGTGGATGGCAACTTT | |
| | GACGATGCTGCCTGGGCTCAGGGACGATGTGACGCCAT | |
| | CGTGCGATCTGGCGCTGGAACCGTCGGACGAACTCAGA | |
| | TTGCCCGACGATCCGCTGCTGTCTGGACCGACGGATGC | |
| | GTGGAGGTCCTGGCTGATTCGGGTAACCGACGACCCTC | |
| | TACTCGACCTCGAGCTAAGGAGTAA | |
| S. cerevisiae | ACAGGCCCCTTTTCCTTTGTCGATATCATGTAATTAGTT | 3 |
| CYC1 | ATGTCACGCTTACATTCACGCCCTCCTCCCACATCCGCT | |
| terminator | CTAACCGAAAAGGAAGGAGTTAGACAACCTGAAGTCTA | |
| | GGTCCCTATTTATTTTTTTTAATAGTTATGTTAGTATTAA | |
| | GAACGTTATTTATATTTCAAATTTTTCTTTTTTTTCTGTA | |
| | CAAACGCGTGTACGCATGTAACATTATACTGAAAACCT | |
| | TGCTTGAGAAGGTTTTGGGACGCTCGAAGGCTTTAATTT | |
| | GC | |
| Y. lipolytica | AGAGACCGGGTTGGCGGCGCATTTGTGTCCCAAAAAAC | 6 |
| TEF1 promoter | AGCCCCAATTGCCCCAATTGACCCCAAATTGACCCAGT | |
| | AGCGGGCCCAACCCCGGCGAGAGCCCCCTTCTCCCCAC | |
| | ATATCAAACCTCCCCCGGTTCCCACACTTGCCGTTAAGG | |
| | GCGTAGGGTACTGCAGTCTGGAATCTACGCTTGTTCAG | |
| | ACTTTGTACTAGTTTCTTTGTCTGGCCATCCGGGTAACC | |
| | CATGCCGGACGCAAAATAGACTACTGAAAATTTTTTTG | |
| | CTTTGTGGTTGGGACTTTAGCCAAGGGTATAAAAGACC | |
| | ACCGTCCCCGAATTACCTTTCCTCTTCTTTTCTCTCTCTC | |
| | CTTGTCAACTCACACCCGAAATCGTTAAGCATTTCCTTC | |
| | TGAGTATAAGAATCATTCAAA | |
| pta or xpk gene | [gene_of_interest] | N/A |
| of interest | | |
| A. | GCGGTTTAGATTTTCCAATTGTAAATATATTACTGTACC | 22 |
| adeninivorans | ATTCTGTACTAAATAACGTGTTTTTTATACTACTTCCTAT | |
| CYC1 | CTATATTCTATATCGTTACTGGCATATATATATCGTTGC | |
| terminator | TGGAGGTCGAAGGTGAAATTTCACTTGCCTTTCTCTTCC | |
| | CCGAGCCGCACGCCGCCATATCGTTATCTTGATTGAGCC | |
| | CGACATCATGATCACTAACTGATTATGCTCCAGCAGGA | |
| | GCAGGTTGTAGAGCTCCAAGATTTGCGAGGAGGTGGAG | |
| | AGAATAGCACGGGACAGAGAGTACGATGG | |

TABLE 9
List of gene sequences used in the construction of Xpk/Pta pathway in
Y. lipolytica strain YB-392.

| | | SEQ ID |
|---|---|---|
| Gene name | Sequence | NO: |
| BsPTA(v1) | ATGGCCGATTTGTTCTCTACCGTTCAAGAAAAAGTTGCT | 23 |
| (CO to | GGTAAGGATGTCAAGATCGTTTTTCCAGAAGGTTTGGA | |
| S. cerevisiae | CGAAAGAATTTTGGAAGCTGTTTCTAAATTGGCCGGTA | |
| using GeneArt) | ACAAGGTTTTGAACCCAATCGTTATTGGTAACGAAAAC | |
| | GAAATTCAAGCCAAGGCCAAAGAATTGAACTTGACTTT | |
| | GGGTGGTGTTAAGATCTACGATCCACATACTTATGAAG | |
| | GTATGGAAGATTTGGTTCAAGCCTTCGTTGAAAGAAGA | |
| | AAAGGTAAGGCTACTGAAGAACAAGCTAGAAAAGCTTT | |
| | GTTAGACGAAAACTACTTCGGTACTATGTTGGTCTACAA | |
| | AGGTTTGGCTGATGGTTTGGTTTCTGGTGCTGCTCATTC | |
| | TACTGCTGATACTGTTAGACCAGCATTGCAAATCATCAA | |
| | GACAAAAGAAGGTGTCAAAAAGACCTCCGGTGTTTTCA | |
| | TTATGGCTAGAGGTGAAGAACAATACGTTTTCGCTGATT | |
| | GCGCTATTAACATTGCTCCAGATTCTCAAGATTTGGCCG | |
| | AAATTGCTATTGAATCTGCTAACACTGCTAAGATGTTCG | |
| | ACATTGAACCTAGAGTTGCTATGTTGTCATTCTCTACAA | |
| | AAGGTTCTGCTAAGTCTGACGAAACTGAAAAGGTTGCT | |
| | GATGCAGTTAAGATCGCTAAAGAAAAAGCTCCAGAATT | |
| | GACCTTGGATGGTGAATTTCAATTTGATGCTGCTTTCGT | |
| | TCCATCCGTTGCTGAAAAAAAAGCACCAGATTCTGAAA | |
| | TCAAGGGTGATGCCAATGTTTTCGTATTCCCATCTTTAG | |
| | AAGCTGGTAACATCGGTTACAAGATTGCTCAAAGATTG | |
| | GGTAACTTTGAAGCTGTTGGTCCAATATTGCAAGGTTTG | |
| | AATATGCCAGTTAACGATTTGTCTAGAGGTTGCAATGC | |
| | AGAAGATGTTTACAACTTGGCTTTGATTACTGCTGCTCA | |
| | AGCTTTGTAA | |
| TsPTA(v1) | ATGTCCATCATCCAAAACATCATCGAAAAGGCCAAGTC | 24 |
| (CO to | CGATAAGAAGAAAATCGTTTTGCCAGAAGGTGCTGAAC | |
| S. cerevisiae | CTAGAACTTTGAAAGCTGCTGAAATCGTCTTGAAAGAA | |
| using GeneArt) | GGTATTGCTGATTTGGTCTTGTTGGGTAACGAAGACGA | |
| | AATTAGAAATGCTGCCAAGGATTTGGATATCTCCAAGG | |
| | CCGAAATTATCGATCCAGTTAAGTCTGAAATGTTCGAC | |
| | AGATACGCTAACGACTTCTACGAATTGAGAAAGAACAA | |
| | GGGTATCACCTTGGAAAAGGCTAGAGAAACCATTAAGG | |
| | ACAACATCTACTTCGGTTGCATGATGGTCAAAGAAGGT | |
| | TACGCTGACGGTTTGGTTTCTGGTGCTATTCATGCTACA | |
| | GCTGATTTGTTAAGACCAGCCTTCCAAATTATCAAAACT | |
| | GCTCCAGGTGCTAAGATCGTCAGTTCATTTTTCATTATG | |
| | GAAGTCCCAAACTGCGAATACGGTGAAAATGGTGTTTT | |
| | TTTGTTCGCTGATTGTGCCGTTAATCCATCTCCAAATGC | |
| | TGAAGAATTGGCTTCCATTGCTGTTCAATCTGCTAATAC | |
| | TGCTAAGAATTTGTTGGGTTTCGAACCTAAGGTTGCCAT | |
| | GTTGTCTTTTTCAACAAAAGGTTCCGCTTCCCATGAATT | |
| | GGTTGATAAGGTTAGAAAGGCTACCGAAATCGCCAAAG | |
| | AATTGATGCCAGATGTTGCTATTGATGGTGAATTACAAT | |
| | TGGATGCTGCCTTGGTAAAAGAAGTTGCTGAATTGAAA | |
| | GCTCCAGGTTCAAAAGTTGCTGGTTGTGCTAATGTTTTG | |
| | ATCTTCCCAGACTTACAAGCTGGTAACATTGGTTACAAG | |
| | TTGGTTCAAAGATTGGCTAAGGCTAATGCCATTGGTCCA | |
| | ATTACTCAAGGTATGGGTGCTCCAGTTAATGATTTGTCT | |
| | AGAGGTTGTTCCTACAGAGATATCGTTGATGTTATTGCT | |
| | ACTACCGCTGTTCAAGCTCAATAA | |
| TsPTA(v2) | ATGTCCATCATCCAGAACATCATCGAGAAGGCCAAGTC | 25 |
| (CO to | TGACAAGAAGAAGATCGTTCTGCCCGAGGGCGCTGAGC | |
| Y. lipolytica | CCCGAACTCTGAAGGCCGCCGAGATCGTGCTGAAGGAA | |
| using GeneArt) | GGCATTGCCGACCTGGTGCTGCTGGGCAACGAGGACGA | |
| | GATCCGAAACGCCGCCAAGGACCTGGACATCTCTAAGG | |
| | CCGAGATCATCGACCCCGTGAAGTCTGAGATGTTCGAC | |
| | CGATACGCCAACGACTTCTACGAGCTGCGAAAGAACAA | |
| | GGGCATCACCCTGGAAAAGGCCCGAGAGACTATCAAGG | |
| | ACAACATCTACTTCGGCTGCATGATGGTCAAGGAAGGC | |
| | TACGCCGACGGCCTGGTGTCTGGCGCCATCCACGCCAC | |
| | CGCCGACCTGCTGCGACCCGCCTTCCAGATCATCAAGA | |
| | CTGCCCCTGGCGCCAAGATCGTGTCCTCGTTCTTCATCA | |
| | TGGAAGTGCCCAACTGCGAGTACGGCGAGAACGGCGTG | |
| | TTCCTGTTCGCCGACTGCGCTGTGAACCCCTCGCCTAAC | |
| | GCCGAGGAACTGGCCTCTATCGCCGTGCAGTCTGCCAA | |
| | CACCGCTAAGAACCTGCTGGGCTTCGAGCCCAAGGTGG | |
| | CCATGCTGTCTTTCTCGACCAAGGGCTCTGCCTCTCACG | |
| | AGCTGGTGGACAAGGTGCGAAAGGCTACCGAGATCGCC | |
| | AAGGAACTGATGCCCGACGTGGCCATCGACGGCGAACT | |
| | GCAGCTGGACGCCGCTCTGGTGAAGGAAGTGGCCGAGC | |
| | TGAAGGCTCCCGGCTCTAAGGTGGCCGGCTGCGCCAAC | |
| | GTGCTGATCTTCCCCGACCTGCAGGCCGGCAACATCGG | |
| | CTACAAGCTGGTGCAGCGACTGGCCAAGGCCAACGCCA | |
| | TCGGACCCATCACTCAAGGCATGGGCGCTCCCGTGAAC | |
| | GACCTGTCTCGAGGCTGCTCTTACCGAGACATCGTGGA | |
| | CGTGATCGCCACCACCGCTGTGCAGGCCCAGTAA | |
| CaXPK(v1) | ATGCAGTCCATCATCGGTAAACACAAGGATGAAGGTAA | 26 |
| (CO to Y. | GATTACCCCTGAATACTTGAAGAAGATTGACGCTTATTG | |
| lipolytica using | GCGAGCTGCCAACTTTATTTCTGTCGGTCAGCTTTACTT | |
| ATGme) | GTTGGACAACCCTTTGTTGCGAGAACCTTTGAAACCTGA | |
| | ACACTTGAAGCGAAAGGTTGTTGGTCACTGGGGTACTA | |
| | TTCCTGGTCAGAACTTCATCTACGCCCACTTGAACCGAG | |
| | TCATCAAAAAGTACGATTTGGACATGATCTACGTTTCTG | |
| | GTCCTGGTCACGGTGGTCAGGTTATGGTTTCTAACTCTT | |
| | ACTTGGACGGTACTTACTCCGAAGTTTACCCTAACGTTT | |
| | CCCGAGATTTGAACGGTTTGAAGAAGTTGTGCAAGCAG | |
| | TTTTCATTCCCTGGTGGTATCTCTTCACACATGGCTCCTG | |
| | AAACTCCTGGTTCTATTAACGAAGGTGGTGAATTGGGTT | |
| | ATTCCTTGGCTCACTCTTTTGGTGCTGTTTTCGATAACCC | |
| | TGATTTGATTACTGCTTGCGTTGTTGGTGATGGTGAAGC | |
| | TGAAACTGGTCCTTTGGCTACATCTTGGCAGGCTAACAA | |
| | ATTTTTGAACCCTGTTACTGATGGTGCCGTTTTGCCTATT | |
| | CTTCACTTGAACGGTTACAAGATCTCCAACCCTACTGTC | |
| | TTGTCTCGAATTCCTAAGGACGAATTGGAAAAGTTCTTC | |
| | GAAGGTAACGGTTGGAAGCCTTACTTTGTTGAAGGTGA | |
| | AGATCCTGAAACCATGCACAAGTTGATGGCTGAAACTT | |
| | TGGATATCGTCACCGAAGAAATCTTGAACATTCAGAAG | |
| | AACGCCCGAGAAAACAACGATTGCTCTCGACCTAAATG | |
| | GCCTATGATCGTTTTGCGAACTCCTAAAGGTTGGACTGG | |
| | TCCTAAATTCGTTGATGGTGTTCCTAACGAAGGTTCTTT | |
| | TCGAGCACACCAGGTTCCTTTGGCAGTTGATCGATACCA | |
| | CACCGAAAACTTGGACCAGTTGGAAGAATGGTTGAAGT | |
| | CTTACAAGCCTGAAGAACTTTTCGACGAAAACTACCGA | |
| | TTGATCCCTGAACTTGAAGAATTGACCCCTAAGGGTAA | |
| | CAAGCGAATGGCTGCTAACTTGCACGCTAACGGTGGTT | |
| | TGTTGCTTCGAGAATTGCGAACCCCTGATTTCCGAGATT | |
| | ACGCTGTTGATGTTCCTACACCTGGTTCAACTGTTAAGC | |
| | AGGATATGATCGAATTGGGTAAATACGTCCGAGATGTC | |
| | GTCAAGTTGAACGAAGATACACGAAACTTCCGAATCTT | |
| | CGGTCCTGACGAAACTATGTCTAACCGATTGTGGGCTGT | |
| | CTTTGAAGGTACTAAGCGACAGTGGTTGTCCGAAATCA | |
| | AAGAACCTAACGACGAATTCTTGTCCAACGATGGTCGA | |
| | ATCGTTGACTCTATGTTGTCTGAACACTTGTGTGAAGGT | |
| | TGGCTTGAAGGTTACTTGTTGACTGGTCGACACGGTTTT | |
| | TTTGCCTCTTACGAAGCTTTCTTGCGAATCGTCGATTCC | |
| | ATGATTACCCAGCACGGTAAATGGTTGAAAGTCACCTC | |
| | TCAGTTGCCTTGGCGAAAGGATATTGCTTCCTTGAACTT | |
| | GATTGCCACCTCTAACGTTTGGCAGCAGGATCACAACG | |
| | GTTATACCCACCAGGACCCTGGTTTGTTGGGTCACATAG | |
| | TTGATAAGAAGCCTGAAATCGTTCGAGCTTATTTGCCTG | |
| | CTGATGCTAACACTTTGTTGGCCGTTTTTGATAAGTGCT | |
| | TGCACACCAAGCACAAGATCAACTTGTTGGTTACCTCTA | |
| | AACACCCTCGACAGCAGTGGCTTACTATGGATCAGGCC | |
| | GTTAAGCACGTTGAACAGGGTATTTCTATTTGGGATTGG | |
| | GCTTCTAACGATAAGGGTCAGGAACCTGATGTTGTTATT | |
| | GCTTCCTGTGGTGATACTCCTACTTTGGAAGCTTTGGCT | |
| | GCTGTTACCATTCTTCACGAACACTTGCCTGAATTGAAG | |
| | GTCCGATTCGTTAACGTTGTTGACATGATGAAGTTGTTG | |
| | CCTGAAAACGAACACCCTCACGGTTTGTCTGATAAGGA | |
| | TTACAACGCTTTGTTCACTACCGATAAGCCTGTTATTTT | |
| | TGCCTTTCACGGTTTCGCCCACTTGATCAACCAGTTGAC | |
| | CTACCACCGAGAAAACCGAAACTTGCACGTTCACGGTT | |
| | ACATGGAAGAAGGTACAATTACTACTCCTTTCGACATG | |
| | CGAGTCCAGAACAAGTTGGACCGATTCAACTTGGTTAA | |
| | GGACGTCGTTGAAAACTTGCCTCAGCTTGGTAACCGAG | |
| | GTGCCCACTTGGTTCAGTTGATGAACGATAAGTTGGTCG | |
| | AACACAACCAGTATATCCGAGAAGTTGGTGAAGATTTG | |
| | CCTGAAATTACCAACTGGCAGTGGCACGTTTGA | |
| CaXPK(v2) | ATGCAGTCCATCATCGGAAAGCACAAGGATGAGGGAA | 27 |
| (manually CO | AGATTACCCCCGAGTACCTCAAGAAGATTGACGCTTAC | |
| to Y. lipolytica | TGGCGAGCTGCCAACTTCATTTCTGTCGGACAGCTCTAC | |
| by replacing | CTCCTCGACAACCCCCTCCTCCGAGAGCCCCTCAAGCCC | |
| all possible | GAGCACCTCAAGCGAAAGGTGGTGGGACACTGGGGAA | |
| codons at | CCATTCCCGGACAGAACTTCATCTACGCCCACCTCAACC | |
| frequency ≤2% | GAGTCATCAAGAAGTACGATCTCGACATGATCTACGTG | |
| using ATGme) | TCTGGACCCGGACACGGAGGACAGGTGATGGTGTCTAA | |
| | CTCTTACCTCGACGGAACCTACTCCGAGGTGTACCCCAA | |
| | CGTGTCCCGAGATCTCAACGGACTCAAGAAGCTCTGCA | |
| | AGCAGTTCTCTTTCCCCGGAGGAATCTCTTCTCACATGG | |
| | CTCCCGAGACCCCCGGATCTATTAACGAGGGAGGAGAG | |
| | CTCGGATACTCCCTCGCTCACTCTTTCGGAGCTGTGTTC | |
| | GATAACCCCGATCTCATTACCGCTTGCGTGGTGGGAGA | |
| | TGGAGAGGCTGAGACCGGACCCCTCGCTACCTCTTGGC | |
| | AGGCTAACAAGTTCCTCAACCCCGTGACCGATGGAGCC | |
| | GTGCTCCCCATTCTCCACCTCAACGGATACAAGATCTCC | |
| | AACCCCACCGTCCTCTCTCGAATTCCCAAGGACGAGCTC | |
| | GAGAAGTTCTTCGAGGGAAACGGATGGAAGCCCTACTT | |
| | CGTGGAGGGAGAGGATCCCGAGACCATGCACAAGCTCA | |
| | TGGCTGAGACCCTCGATATCGTCACCGAGGAGATCCTC | |
| | AACATTCAGAAGAACGCCCGAGAGAACAACGATTGCTC | |
| | TCGACCCAAGTGGCCCATGATCGTGCTCCGAACCCCCA | |
| | AGGGATGGACCGGACCCAAGTTCGTGGATGGAGTGCCC | |
| | AACGAGGGATCTTTCCGAGCTCACCAGGTGCCCCTCGC | |
| | TGTGGATCGATACCACACCGAGAACCTCGACCAGCTCG | |
| | AGGAGTGGCTCAAGTCTTACAAGCCCGAGGAGCTCTTC | |
| | GACGAGAACTACCGACTCATCCCCGAGCTCGAGGAGCT | |
| | CACCCCCAAGGGAAACAAGCGAATGGCTGCTAACCTCC | |
| | ACGCTAACGGAGGACTCCTCCTCCGAGAGCTCCGAACC | |
| | CCCGATTTCCGAGATTACGCTGTGGATGTGCCCACCCCC | |
| | GGATCTACCGTGAAGCAGGATATGATCGAGCTCGGAAA | |
| | GTACGTCCGAGATGTCGTCAAGCTCAACGAGGATACCC | |
| | GAAACTTCCGAATCTTCGGACCCGACGAGACCATGTCT | |
| | AACCGACTCTGGGCTGTCTTCGAGGGAACCAAGCGACA | |
| | GTGGCTCTCCGAGATCAAGGAGCCCAACGACGAGTTCC | |
| | TCTCCAACGATGGACGAATCGTGGACTCTATGCTCTCTG | |
| | AGCACCTCTGTGAGGGATGGCTCGAGGGATACCTCCTC | |
| | ACCGGACGACACGGATTCTTCGCCTCTTACGAGGCTTTC | |
| | CTCCGAATCGTCGATTCCATGATTACCCAGCACGGAAA | |
| | GTGGCTCAAGGTCACCTCTCAGCTCCCCTGGCGAAAGG | |
| | ATATTGCTTCCCTCAACCTCATTGCCACCTCTAACGTGT | |
| | GGCAGCAGGATCACAACGGATACACCCACCAGGACCCC | |
| | GGACTCCTCGGACACATTGTGGATAAGAAGCCCGAGAT | |
| | CGTGCGAGCTTACCTCCCCGCTGATGCTAACACCCTCCT | |
| | CGCCGTGTTCGATAAGTGCCTCCACACCAAGCACAAGA | |
| | TCAACCTCCTCGTGACCTCTAAGCACCCCCGACAGCAGT | |
| | GGCTCACCATGGATCAGGCCGTGAAGCACGTGGAGCAG | |
| | GGAATTTCTATTTGGGATTGGGCTTCTAACGATAAGGG | |
| | ACAGGAGCCCGATGTGGTGATTGCTTCCTGTGGAGATA | |
| | CCCCCACCCTCGAGGCTCTCGCTGCTGTGACCATTCTCC | |
| | ACGAGCACCTCCCCGAGCTCAAGGTCCGATTCGTGAAC | |
| | GTGGTGGACATGATGAAGCTCCTCCCCGAGAACGAGCA | |
| | CCCCCACGGACTCTCTGATAAGGATTACAACGCTCTCTT | |
| | CACCACCGATAAGCCCGTGATTTTCGCCTTCCACGGATT | |
| | CGCCCACCTCATCAACCAGCTCACCTACCACCGAGAGA | |
| | ACCGAAACCTCCACGTGCACGGATACATGGAGGAGGGA | |
| | ACCATTACCACCCCCTTCGACATGCGAGTCCAGAACAA | |
| | GCTCGACCGATTCAACCTCGTGAAGGACGTCGTGGAGA | |
| | ACCTCCCCCAGCTCGGAAACCGAGGAGCCCACCTCGTG | |
| | CAGCTCATGAACGATAAGCTCGTCGAGCACAACCAGTA | |
| | CATCCGAGAGGTGGGAGAGGATCTCCCCGAGATTACCA | |
| | ACTGGCAGTGGCACGTGTGA | |

CO-Codon optimization; XPK-Phosphoketolase; PTA-Phosphotransacetylase

### Methods

### XPK and PTA Gene Expression

To identify functional XPK and PTA genes, expression cassettes were transformed into the desired Y. lipolytica strains as a part of a linear integrated expression construct or replicating plasmid composed of the genetic parts listed in Tables 6 and 7. For replicating plasmids, 100 ng of undigested plasmid was used in the transformation mix. To assemble the Xpk/Pta/Δpfk1 pathway, NS1047 and subsequent intermediate strains were transformed with linear constructs containing XPK or PTA and positive and negative marker expression cassettes (Table 8). Transformants were selected on antibiotic plates and screened for the highest performance using appropriate enzymatic, lipid and growth assays. Tables 5 and 10 describe the screening steps used to construct NS1475 and NS1656-57, respectively. To eliminate the marker cassette in these strains, the chosen isolates were grown on YPD agar plates without selection for one day to allow for survival of cells that naturally excised the cassette by recombination between the identical copies of the Y. lipolytica TEF1 promoter driving expression of thymidine kinase and the gene of interest in the integration cassette. Subsequent plating on YPD agar containing 30 μM 5-fluoro-2′-deoxyuridine (FUDR) counter-selects for the thymidine kinase gene. FUDR-resistant isolates were screened by confirmation of reversion to nourseothricin sensitivity to identify marker-less strains.

### Cell-Free Extract Preparation and Enzymatic Assays

Strains were grown in 5 mL YPD or YPG overnight at 30° C. The cells were pelleted by centrifugation and after a wash with autoclaved water, were pelleted again. The pellets were resuspended in lysis buffer Y-PER™ plus (Thermo Scientific) per the manufacturer's instructions. Protease inhibitor cocktail (Sigma Aldrich) was added (5 μL for every 1 mL of the lysis buffer used) and 0.5-mm glass beads were added at an equal volume to the cell pellet. The cells were homogenized in a FastPrep-24™ 5G (MP biomedicals) (3 cycles of 5.5 m/s for 30 s, with 5 min resting on ice in between runs). The homogenized cell lysates were centrifuged at 10,000 rpm for 10 min at 4° C. and the supernatants were stored on ice for immediate use in enzymatic assays. Total protein concentrations were determined by the Pierce™ Coomassie (Bradford) Protein Assay Kit (Thermo Scientific).

Phosphotransacetylase activity was quantified using Ellman's thiol reagent, 5,5′-dithiobis-(2-nitrobenzoic acid) (DTNB) which reacts with Coenzyme A to form a mercaptide ion measurable at 412 nm [51], with an extinction coefficient of 13.5 mM⁻¹ cm⁻¹. The 1-mL reaction mixture contained 100 mM Tris-HCl (pH 7.2), 5 mM MgCl₂, 5 mM KH₂PO₄, 0.1 mM DTNB, and 0.1 mM acetyl-CoA [52]. 10-40 μL of cell-free extract was mixed with the assay ingredients, with acetyl-CoA added at the end to start the reaction. Specific activity measurements were calculated.

Phosphoketolase activity was measured using a ferric hydroxamate assay on crude cell-free extracts [53]. The 200 μL reaction mixture contained 0.5 mM thiamine pyrophosphate (TPP), 1 mM DTT, 5 mM MgCl₂, 50 mM morpholine ethane sulfonic acid (MES) buffer (pH 5.5 for all kinetic studies), 333 mM sodium phosphate substrate and 333 mM of either fructose 6-phosphate or ribose 5-phosphate as substrate. Ribose 5-phosphate which is converted to X5P by endogenous enzymes in cell-free extract, was used to measure phosphoketolase activity indirectly [54,55]. 20-80 μL of cell-free extract was used to initiate the reaction, and the mixture was incubated at 37° C. for 15-30 min. 100 μL of 2 M hydroxylamine hydrochloride (pH 7.0) was added and incubated at room temperature for 10 min to stop the reaction. 600 μL of a 1:1 mixture of 2.5% FeCl₃ in 2 N HCl and 10% trichloroacetic acid was added. The final reaction step results in the formation of the ferric-hydroxamate complex, which was measured spectrophotometrically at 540 nm [56]. For specific activity measurements, reactions were stopped at 5-minute intervals and ΔAbs/min was calculated.

### Codon Optimizations

BsPTA(v1) and TsPTA(v1) genes were codon optimized to S. cerevisiae using the GeneArt Gene Synthesis service (ThermoFisher Scientific). TsPTA(v2) and CaXPK(v1) were codon optimized to Y. lipolytica using GeneArt Gene Synthesis service and the open source web application ATGme [57], respectively. CaXPK(v2) was codon optimized using the ATGme web application by manual replacement of all possible codons in the gene present at a frequency≤2% with their higher frequency counterparts. All the gene sequences used in the strain engineering are listed in Table 9.

### Glucose Batch Fermentation in 1-L Bioreactors

Frozen working-stocks of strains were patched onto a YPD plate and grown overnight at 30° C. A 10 μL loopful of cells was removed from each plate and used to inoculate separate 250 mL baffled Erlenmeyer flasks with 50 mL of lipid production media. Inoculum flasks were cultured overnight at 30° C. with constant agitation of 200 rpm in a New Brunswick 126 incubator shaker, whereupon the OD₆₀₀ was measured. A volume of each flask culture required to initiate its corresponding 1 L bioreactors at a T₀ cell density of 0.4 OD₆₀₀ was transferred to separate sterile conical tubes. Each conical tube was then brought to 50 mL with sterile diH₂O and centrifuged at 4000 rpm for 3 minutes in an Eppendorf 5810 R centrifuge. The supernatant was decanted and the cells were then resuspended in 50 mL sterile diH₂O. 25 mL of this washed inoculum was used to inoculate each of two 1 L working volume bioreactors (Dasgip, 1.2 L vessels) with medium consisting of: glucose (150 g/L), (NH₄)₂SO₄ (0.5 g/L), KH₂PO₄ (4 g/L), yeast extract (3 g/L), Amberferm 4500 (50 mg/L), MgSO₄·7H₂O (2 g/L), D-biotin (1 mg/L), thiamine hydrochloride (12 mg/L), ZnSO₄·7H₂O (20 mg/L), MnSO₄·H₂O (180 mg/L), CoCl₂·6H₂O (0.03 mg/L), CuSO₄·5H₂O (0.2 mg/L), Na₂MoO₄·2H₂O (160 mg/L), CaCl₂·6H₂O (800 mg/L), FeCl₃·6H₂O (75 mg/L), H₃BO₃ (40 mg/L). Process parameters included a pH control at 3.5 automatically adjusted with 10 N sodium hydroxide, a temperature of 30° C., aeration at 0.3 vvm air, and agitation controlled at 1000 rpm. A sample of 10 mL was taken from each culture once per day. The samples were stored at 4° C. after each harvest until analyzed. For all time-points, broth analysis was conducted via HPLC. Total dry cell weight (DCW) and total lipid content were measured gravimetrically by a two-phase solvent extraction. Cell-specific lipid productivities were calculated once the strains reached lipogenesis and their growth had slowed (day 2-day 5).

### Gravimetric Measurement of Dry Cell Weight (DCW) and Total Lipid Content Using a Two-Phase Solvent Extraction

Broth volume from each harvested culture sample was added to a separate pre-weighed 2 mL screw-cap microfuge tube (USA Scientific, 1420-8799) to achieve a dried cell mass between 15-20 mg. Samples were washed twice with deionized H₂O and centrifuged at 21130×g for 2 minutes. Pelleted cells were then resuspended in 200 μL of deionized H₂O, frozen at −80° C. for 30 minutes, and freeze-dried overnight. Each tube was weighed to obtain the DCW. To each freeze-dried sample and three blank microfuge tubes, 400 mg of glass beads (Sigma, G8772) and 400 μL of a 1.5:1 CPME:MeOH (Cyclopentyl methyl ether:Methanol) solution was added. Under maximum agitation, samples were then bead-beaten (BioSpec Mini-BeadBeater 8) for 2 minutes and allowed to cool to room temperature. After having cooled, 640 μL of CPME followed by 640 μL of 10% (w/v) CaCl₂·6H₂O were added to each sample and vortexed. Samples were then centrifuged for 2 minutes at 21130×g, creating two distinct layers. 660 μL (75% of calculated volume) of the top layer, containing CPME and lipid, was transferred to a pre-weighed glass vial. Dispensed samples were evaporated under compressed air until no visual solvent remained and then lyophilized overnight for total solvent removal. The remaining lipid was weighed and corrected by subtracting the average residual mass measured in the blank samples.

### HPLC Analysis

The extracellular concentrations of glucose, citrate and polyols (erythritol, arabitol and mannitol) were determined by high-performance liquid chromatography analysis. To that end, a 1 mL broth sample was filtered through a 0.2 mm syringe filter and analyzed using an Aminex HPX-87H column (300 mm×7.8 mm) (Bio-Rad) on an Agilent 1260 Infinity II HPLC equipped with a refraction index detector (Agilent Technologies). The column was eluted with 5 mM H₂SO₄ at a flow rate of 0.6 mL min⁻¹ at 45° C. for 25 min. The eluents were determined by comparing peak retention times to those of known standard substances, and the amounts were quantified by comparing the peak area of the analyte to the peak area of the standard substance at known concentrations.

### Example 4—Confirming Improved Lipid Production with Xpk/Pta/Δpfk1 Genotype

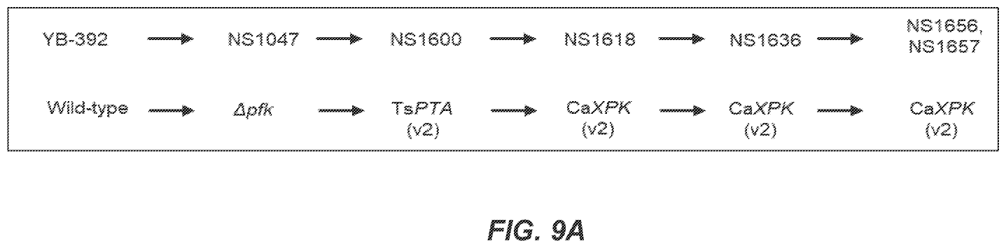

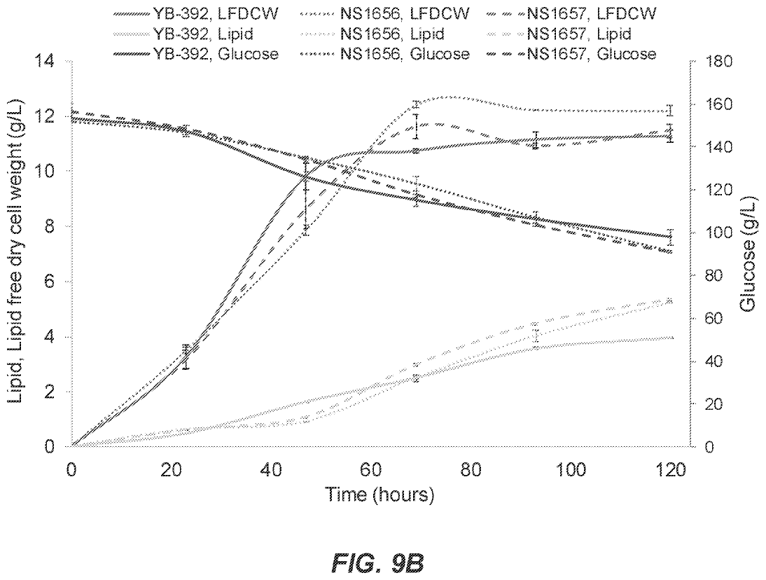

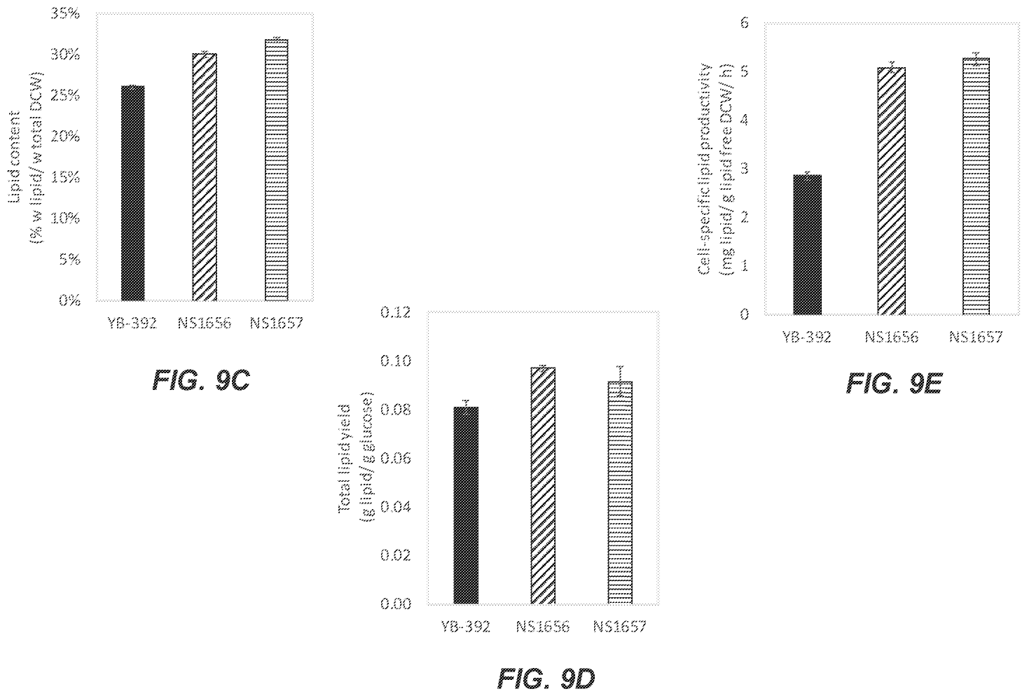

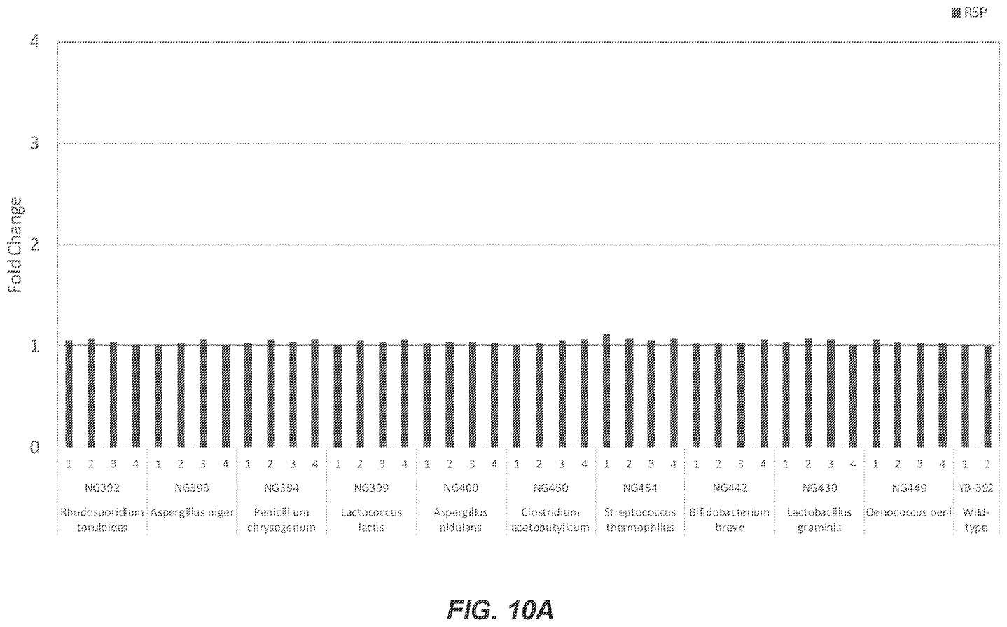

To confirm that the Xpk/Pta pathway was directly responsible for restoring growth and improving lipid production from glucose in Δpfk1, the pathway was reconstructed in NS1047 using only TsPTA(v2) and CaXPK(v2) (FIG. 9A) in order to eliminate the possibility that the nine rounds of transformation involved in constructing NS1475 resulted in unintentional contributions to the strain's phenotype. As before, Pta and Xpk activity, growth on glucose, and lipid production were monitored at each engineering step (Table 10). One copy of TsPTA(v2) and three copies of CaXPK(v2) were required to restore growth and lipid accumulation to YB-392 levels reducing the number of engineering steps to five instead of nine. Despite the usage of a more active XPK gene sequence, the pathway was again Xpk-limited and required more copies of XPK than PTA to achieve the optimal phenotype.

TABLE 10
Steps involved in the construction of strains NS1656 and NS1657

| | | # of | |
|---|---|---|---|
| | | isolates | |
| Strains | Genotype | screened | Screening assay |
| NS1600 | Δpfk1, TsPTA(v2) | 10 | DTNB assay |
| NS1618 | Δpfk1, TsPTA(v2), | 12 | Ferric |
| | CaXPK(v2) | | hydroxamate assay |
| NS1636 | Δpfk1, TsPTA(v2), | 76 | Growth and lipid |
| | 2xCaXPK(v2) | | assay on lipid |
| | | | production media |
| NS1656-57 | Δpfk1, TsPTA(v2), | 52 | Growth and lipid |
| | 3xCaXPK(v2) | | assay on lipid |
| | | | production media |

The two best-performing strains at the end of this engineering strategy (NS1656 and NS1657, FIG. 9A) were further characterized in 1-L batch fermentations alongside YB-392. Under the same fermentation conditions, all three strains attained similar lipid-free biomass (FIG. 9B). As was the case with NS1475, the Xpk/Pta/Δpfk1 strains outperformed YB-392 in lipid yield and cell-specific lipid productivity (FIGS. 9C and 9D). A slight improvement in lipid content was also observed (FIG. 9E). Overall, all three engineered strains showed an improvement in total lipid yield ranging from 13.3%-19.6% and an improvement in cell-specific productivity ranging from 41%-78% over YB-392.

Optimized phosphoketolase and phosphotransacetylase genes were expressed in a phosphofructokinase-deficient Δpfk1 strain to demonstrate the use of Xpk/Pta pathway in improving lipid production. The engineered strains recorded up to 19% higher total lipid yield and up to 78% higher cell-specific productivity compared to the wild-type strain. Such improvements in bioprocess metrics make lipid production in Y. lipolytica more suitable for industrial applications. Since the Xpk/Pta pathway essentially improves acetyl-CoA production, this pathway can be used to improve bioprocess metrics of other acetyl-CoA derived products including fatty alcohols, sterols, alkenes/alkanes, isoprenoids etc. [48]. The theoretical improvement in yield makes the Xpk/Pta pathway a compelling technology for large scale, commodity fermentation in the biofuel and biochemical industries.

### Example 5—Characterization of Xpk Activity from Various Organisms

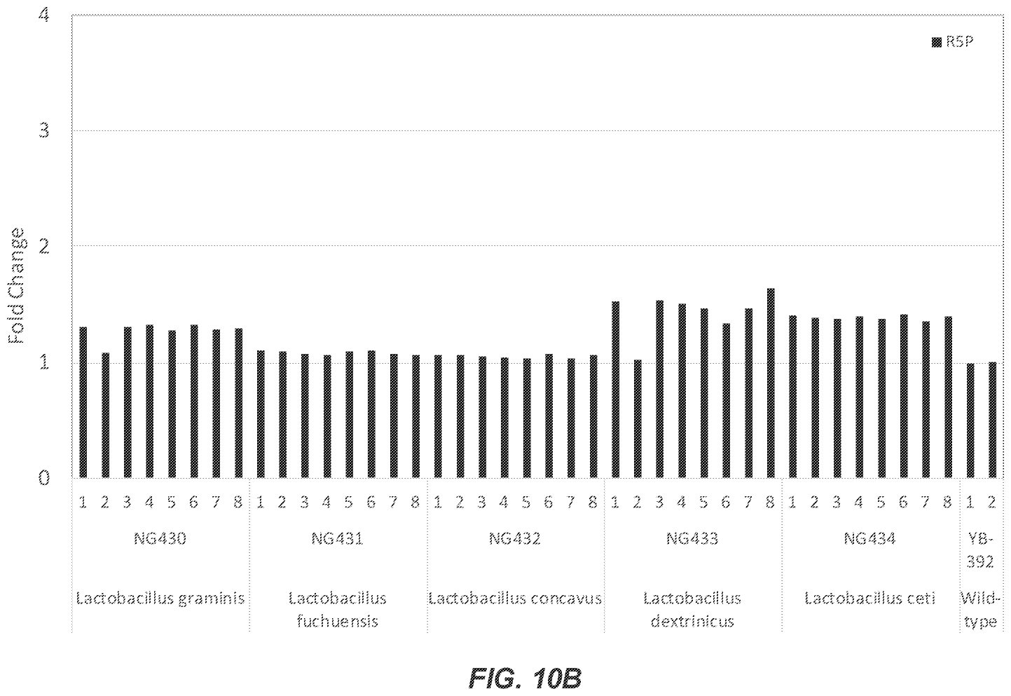

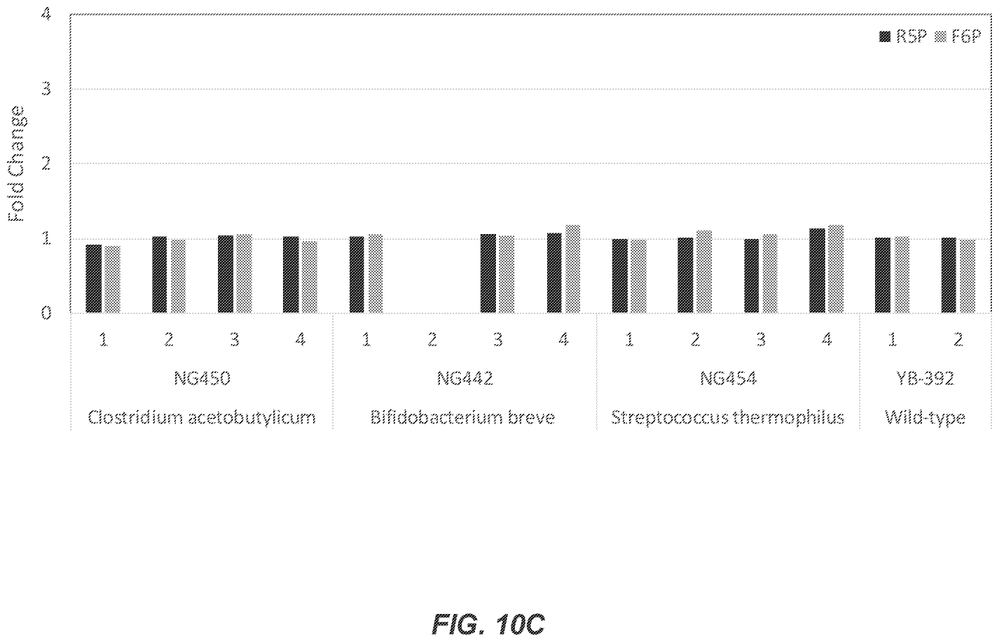

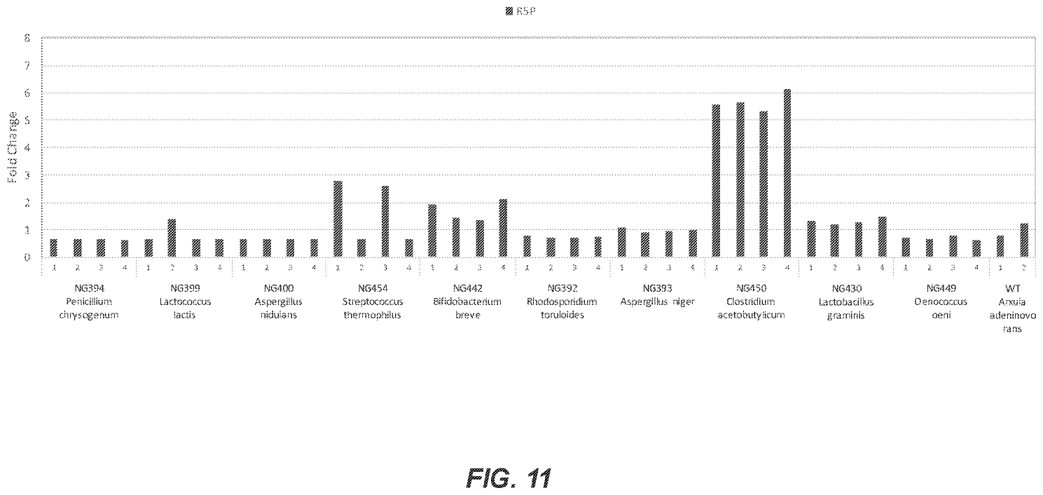

Xpk genes from various source organisms were codon optimized to either S. cerevisiae or Y. lipolytica (using GeneArt, ATGme, or manually codon optimized) and expressed in Y. lipolytica strain YB-392 (FIGS. 4, 7B, and FIGS. 10A-10C) and A. adeninivorans (FIG. 11) as described in Examples 1-4. Only Xpk from Clostridium acetobutylicum showed significant phosphoketolase activity in both Y. lipolytica (CaXPK(v1) and CaXPK(v2)) and A. adeninivorans (NG450).

All of the methods disclosed and claimed herein can be made and executed without undue experimentation in light of the present disclosure. While the compositions and methods of this invention have been described in terms of certain embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, and those cited herein, to the extent that they provide exemplary procedural or other details supplementary to those set forth herein, are specifically incorporated herein by reference.
1. Donot F, Fontana A, Baccou J C, Strub C, Schorr-Galindo S. Single cell oils (SCOs) from oleaginous yeasts and moulds: Production and genetics. Biomass and Bioenergy. 2014 September; 68:135-50.
2. Koh L P, Wilcove D S. Is oil palm agriculture really destroying tropical biodiversity? Conservation Letters. 2008; 1(2):60-4.
3. Mendes-Oliveira A C, Peres C A, Maués P C R de A, Oliveira G L, Mineiro I G B, Maria S L S de, et al. Oil palm monoculture induces drastic erosion of an Amazonian forest mammal fauna. PLOS ONE. 2017 Nov. 8; 12(11):e0187650.
4. Beopoulos A, Cescut J, Haddouche R, Uribelarrea J-L, Molina-Jouve C, Nicaud J-M. Yarrowia lipolytica as a model for bio-oil production. Progress in Lipid Research. 2009 November; 48(6):375-87.
5. Pinzi S, Pilar dorado M. 4—Feedstocks for advanced biodiesel production. In: Luque R, Melero J A, editors. Advances in Biodiesel Production [Internet]. Woodhead Publishing; 2012 [cited 2020 Jan. 2]. p. 69-90. (Woodhead Publishing Series in Energy). Available from: http://www.sciencedirect.com/science/article/pii/B9780857091178500046
6. Nicaud J-M. Yarrowia lipolytica. Yeast. 2012 October; 29(10):409-18.
7. Ratledge C, Wynn J P. The biochemistry and molecular biology of lipid accumulation in oleaginous microorganisms. Adv Appl Microbiol. 2002; 51:1-51.
8. Tai M, Stephanopoulos G. Engineering the push and pull of lipid biosynthesis in oleaginous yeast Yarrowia lipolytica for biofuel production. Metabolic Engineering. 2013 January; 15:1-9.
9. Blazeck J, Hill A, Liu L, Knight R, Miller J, Pan A, et al. Harnessing Yarrowia lipolytica lipogenesis to create a platform for lipid and biofuel production. Nature Communications [Internet]. 2014 December [cited 2018 Jun. 17]; 5(1). Available from: http://www.nature.com/articles/ncomms4131
10. Friedlander J, Tsakraklides V, Kamineni A, Greenhagen E H, Consiglio A L, MacEwen K, et al. Engineering of a high lipid producing Yarrowia lipolytica strain. Biotechnol Biofuels. 2016 Mar. 31; 9(1):77.
11. Mlícková K, Roux E, Athenstaedt K, d'Andrea S, Daum G, Chardot T, et al. Lipid accumulation, lipid body formation, and acyl coenzyme A oxidases of the yeast Yarrowia lipolytica. Applied and environmental microbiology. 2004 July; 70(7):3918-24.
12. Tsakraklides V, Kamineni A, Consiglio A L, MacEwen K, Friedlander J, Blitzblau H G, et al. High-oleate yeast oil without polyunsaturated fatty acids. Biotechnol Biofuels. 2018; 11:131.
13. Beopoulos A, Nicaud J-M, Gaillardin C. An overview of lipid metabolism in yeasts and its impact on biotechnological processes. Appl Microbiol Biotechnol. 2011 May; 90(4): 1193-206.
14. Dulermo T, Nicaud J-M. Involvement of the G3P shuttle and β-oxidation pathway in the control of TAG synthesis and lipid accumulation in Yarrowia lipolytica. Metabolic Engineering. 2011 September; 13(5):482-91.
15. Wasylenko T M, Ahn W S, Stephanopoulos G. The oxidative pentose phosphate pathway is the primary source of NADPH for lipid overproduction from glucose in Yarrowia lipolytica. Metabolic Engineering. 2015 July; 30:27-39.
16. Ratledge C. The role of malic enzyme as the provider of NADPH in oleaginous microorganisms: a reappraisal and unsolved problems. Biotechnology Letters. 2014 August; 36(8): 1557-68.
17. Mitchell A G, Martin C E. A novel cytochrome b5-like domain is linked to the carboxyl terminus of the Saccharomyces cerevisiae delta-9 fatty acid desaturase. J Biol Chem. 1995 Dec. 15; 270(50):29766-72.
18. Beopoulos A, Mrozova Z, Thevenieau F, Le Dall M-T, Hapala I, Papanikolaou S, et al. Control of lipid accumulation in the yeast Yarrowia lipolytica. Applied and Environmental Microbiology. 2008 Dec. 15; 74(24): 7779-89.
19. Qiao K, Imam Abidi S H, Liu H, Zhang H, Chakraborty S, Watson N, et al. Engineering lipid overproduction in the oleaginous yeast Yarrowia lipolytica. Metabolic Engineering. 2015 May; 29:56-65.
20. Liu L, Pan A, Spofford C, Zhou N, Alper H S. An evolutionary metabolic engineering approach for enhancing lipogenesis in Yarrowia lipolytica. Metabolic Engineering. 2015 May; 29:36-45.
21. Friedlander J, Tsakraklides V, Kamineni A, Greenhagen E H, Consiglio A L, MacEwen K, et al. Engineering of a high lipid producing Yarrowia lipolytica strain. Biotechnol Biofuels. 2016; 9:77.
22. Kamineni A, Shaw J. Engineering triacylglycerol production from sugars in oleaginous yeasts. Curr Opin Biotechnol. 2020 Jan. 25; 62:239-47.
23. Zhang H, Zhang L, Chen H, Chen Y Q, Chen W, Song Y, et al. Enhanced lipid accumulation in the yeast Yarrowia lipolytica by over-expression of ATP:citrate lyase from Mus musculus. Journal of Biotechnology. 2014 December; 192:78-84.
24. Beopoulos A, Haddouche R, Kabran P, Dulermo T, Chardot T, Nicaud J-M. Identification and characterization of DGA2, an acyltransferase of the DGAT1 acyl-CoA:diacylglycerol acyltransferase family in the oleaginous yeast Yarrowia lipolytica. New insights into the storage lipid metabolism of oleaginous yeasts. Applied Microbiology and Biotechnology. 2012 February; 93(4):1523-37.
25. Blazeck J, Liu L, Knight R, Alper H S. Heterologous production of pentane in the oleaginous yeast Yarrowia lipolytica. Journal of Biotechnology. 2013 June; 165(3-4):184-94.
26. Bhutada G, Kavšček M, Ledesma-Amaro R, Thomas S, Rechberger G N, Nicaud J-M, et al. Sugar versus fat: elimination of glycogen storage improves lipid accumulation in Yarrowia lipolytica. FEMS Yeast Research [Internet]. 2017 May 1 [cited 2018 Jun. 17]; 17(3). Available from: https://academic.oup.com/femsyr/article/doi/10.1093/femsyr/fox020/3798535
27. van Rossum H M, Kozak B U, Pronk J T, van Maris A J A. Engineering cytosolic acetyl-coenzyme A supply in Saccharomyces cerevisiae: Pathway stoichiometry, free-energy conservation and redox-cofactor balancing. Metabolic Engineering. 2016 July; 36:99-115.
28. Qiao K, Wasylenko T M, Zhou K, Xu P, Stephanopoulos G. Lipid production in Yarrowia lipolytica is maximized by engineering cytosolic redox metabolism. Nature Biotechnology. 2017 February; 35(2):173-7.
29. Niehus X, Crutz-Le Coq A-M, Sandoval G, Nicaud J-M, Ledesma-Amaro R. Engineering Yarrowia lipolytica to enhance lipid production from lignocellulosic materials. Biotechnol Biofuels [Internet]. 2018 Jan. 22 [cited 2019 Dec. 10]; 11. Available from: https://www.ncbi.nlm.nih.gov/pmc/articles/PMC5776775/30.
30. Ku J T, Chen A Y, Lan E I. Metabolic engineering design strategies for increasing acetyl-CoA flux. Metabolites. 2020 Apr. 23; 10(4).
31. Lin P P, Jaeger A J, Wu T-Y, Xu S C, Lee A S, Gao F, et al. Construction and evolution of an Escherichia coli strain relying on nonoxidative glycolysis for sugar catabolism Proc Natl Acad Sci USA. 2018 03; 115(14):3538-46.
32. Sonderegger M, Schumperli M, Sauer U. Metabolic engineering of a phosphoketolase pathway for pentose catabolismin Saccharomyces cerevisiae. Appl Environ Microbiol. 2004 May; 70(5):2892-7.
33. Kocharin K, Siewers V, Nielsen J. Improved polyhydroxybutyrate production by Saccharomyces cerevisiae through the use of the phosphoketolase pathway. Biotechnol Bioeng. 2013 August; 110(8):2216-24.
34. de Jong B W, Shi S, Siewers V, Nielsen J. Improved production of fatty acid ethyl esters in Saccharomyces cerevisiae through up-regulation of the ethanol degradation pathway and expression of the heterologous phosphoketolase pathway. Microb Cell Fact. 2014 Mar. 12; 13(1):39.
35. Meadows A L, Hawkins K M, Tsegaye Y, Antipov E, Kim Y, Raetz L, et al. Rewriting yeast central carbon metabolism for industrial isoprenoid production. Nature. 2016 Sep. 29; 537(7622):694-7.
36. Wang Y, San K-Y, Bennett GN. Improvement of NADPH bioavailability in Escherichia coli through the use of phosphofructokinase deficient strains. Appl Microbiol Biotechnol. 2013 August; 97(15):6883-93.
37. Jacoby J, Hollenberg C P, Heinisch J J. Transaldolase mutants in the yeast Kluyveromyces lactis provide evidence that glucose can be metabolized through the pentose phosphate pathway. Mol Microbiol. 1993 November; 10(4):867-76.
38. Boles E, Lehnert W, Zimmermann F K. The role of the NAD-dependent glutamate dehydrogenase in restoring growth on glucose of a Saccharomyces cerevisiae phosphoglucose isomerase mutant. Eur J Biochem. 1993 Oct. 1; 217(1):469-77.
39. Overkamp K M, Bakker B M, Steensma H Y, van Dijken J P, Pronk J T. Two mechanisms for oxidation of cytosolic NADPH by Kluyveromyces lactis mitochondria. Yeast. 2002 July; 19(10):813-24.
40. Quarterman J, Slininger P J, Kurtzman C P, Thompson S R, Dien B S. A survey of yeast from the Yarrowia clade for lipid production in dilute acid pretreated lignocellulosic biomass hydrolysate. Appl Microbiol Biotechnol. 2017 April; 101(8):3319-34.
41. Flores C-L, Martinez-Costa, O H, Sanchez, V, Gancedo, C, Aragon, J J. The dimorphic yeast Yarrowia lipolytica possesses an atypical phosphofructokinase: characterization of the enzyme and its encoding gene. Microbiology. 2005 May 1; 151(5): 1465-74.
42. Canonaco F, Hess T A, Heri S, Wang T, Szyperski T, Sauer U. Metabolic flux response to phosphoglucose isomerase knock-out in Escherichia coli and impact of overexpression of the soluble transhydrogenase UdhA. FEMS Microbiol Lett. 2001 Nov. 13; 204(2):247-52.
43. Kerscher S J, Okun J G, Brandt U. A single external enzyme confers alternative NADH:ubiquinone oxidoreductase activity in Yarrowia lipolytica. J Cell Sci. 1999 July; 112 (Pt 14):2347-54.
44. Mansour S, Beckerich J M, Bonnarme P. Lactate and amino acid catabolismin the cheese-ripening yeast Yarrowia lipolytica. Appl Environ Microbiol. 2008 November; 74(21):6505-12.
45. Ochoa-Estopier A, Bouscaut J, Morin N, Trouilh L, Le Berre V, Loux V, et al. Yarrowia lipolytica biolipid production during D-stat setup. [Internet]. GEO. 2020 Available on the World Wide Web at ncbi.nlm.nih.gov/geo/query/acc.cgi?acc-GSE35447
46. Ochoa-Estopier A, Bouscaut J, Morin N, Trouilh L, Le Berre V, Loux V, et al. Yarrowia lipolytica biolipid production during D-stat setup. [Part 1] [Internet]. GEO. 2020 [cited 2020 Feb. 25]. Available on the World Wide Web at ncbi.nlm.nih.gov/geo/query/acc.cgi?acc-GSE35445
47. Ochoa-Estopier A, Bouscaut J, Morin N, Trouilh L, Le Berre V, Loux V, et al. Yarrowia lipolytica biolipid production during D-stat setup. [Part 2] [Internet]. GEO. 2020 [cited 2020 Feb. 25]. Available on the World Wide Web at ncbi.nlm.nih.gov/geo/query/acc.cgi?acc-GSE35446
48. Nielsen J. Synthetic Biology for Engineering Acetyl Coenzyme A Metabolism in Yeast. mBio [Internet]. 2014 Dec. 31 [cited 2020 Dec. 17]; 5(6). Available on the World Wide Web at mbio.asm.org/content/5/6/e02153-14
49. Tsakraklides V, Brevnova E, Stephanopoulos G, Shaw AJ. Improved gene targeting through cell cycle synchronization. Galli A, editor. PLOS ONE. 2015 Jul. 20; 10(7):e0133434.
50. Verduyn C, Postma E, Scheffers W A, Van Dijken J P. Effect of benzoic acid on metabolic fluxes in yeasts: a continuous-culture study on the regulation of respiration and alcoholic fermentation. Yeast. 1992 July; 8(7):501-17.
51. Srere P A, Brazil H, Gonen L. The citrate condensing enzyme of pigeon breast muscle and moth flight muscle. Acta chem scand. 1963; 17(Suppl 1): 129-134.
52. Bock A K, Glasemacher J, Schmidt R, Schönheit P. Purification and characterization of two extremely thermostable enzymes, phosphate acetyltransferase and acetate kinase, from the hyperthermophilic eubacterium Thermotoga maritima. J Bacteriol. 1999 March; 181(6): 1861-7.
53. Lipmann F A, Tuttle L C. A specific micromethod for the determination of acyl phosphates. In 1945.
54. Bogorad I W, Lin T-S, Liao J C. Synthetic non-oxidative glycolysis enables complete carbon conservation. Nature. 2013 Oct. 31; 502(7473):693-7.
55. Bergman A, Siewers V, Nielsen J, Chen Y. Functional expression and evaluation of heterologous phosphoketolases in Saccharomyces cerevisiae. AMB Express. 2016 December; 6(1):115.
56. Glenn K, Ingram-Smith C, Smith KS. Biochemical and kinetic characterization of xylulose 5-phosphate/fructose 6-phosphate phosphoketolase 2 (Xfp2) from Cryptococcus neoformans. Eukaryot Cell. 2014 May; 13(5):657-63.
57. Daniel E, Onwukwe G U, Wierenga R K, Quaggin S E, Vainio S J, Krause M. ATGme: Open-source web application for rare codon identification and custom DNA sequence optimization. BMC Bioinformatics [Internet]. 2015 Sep. 21 [cited 2020 Jan. 3]; 16(1).

What is claimed is:

1. A recombinant yeast cell, comprising:
(a) a first exogenous nucleic acid sequence encoding a phosphoketolase protein comprising a sequence having at least 90% sequence identity to SEQ ID NO:28; and
(b) a second exogenous nucleic acid sequence encoding a phosphotransacetylase protein.

2. The recombinant yeast cell of claim 1, wherein the phosphoketolase protein and the phosphotransacetylase protein are expressed by the recombinant yeast cell as separate proteins.

3. The recombinant yeast cell of claim 1, wherein the phosphoketolase protein and the phosphotransacetylase protein are expressed by the recombinant yeast cell as a fusion protein.

4. The recombinant yeast cell of claim 3, wherein the fusion protein comprises the phosphoketolase protein attached to the phosphotransacetylase protein via a linker.

5. The recombinant yeast cell of any of claims 1-4, wherein the phosphoketolase protein comprises a sequence having at least 95% identity to SEQ ID NO:28.

6. The recombinant yeast cell of claim 5, wherein the phosphoketolase protein comprises a sequence having at least 98% identity to SEQ ID NO:28.

7. The recombinant yeast cell of claim 6, wherein the phosphoketolase protein comprises a sequence having at least 99% identity to SEQ ID NO:28.

8. The recombinant yeast cell of claim 7, wherein the phosphoketolase protein comprises SEQ ID NO:28.

9. The recombinant yeast cell of any of claims 1-8, wherein the first exogenous nucleic acid sequence comprises a sequence having at least 75% identity to SEQ ID NO:26.

10. The recombinant yeast cell of claim 9, wherein the first exogenous nucleic acid sequence comprises a sequence having at least 80% identity to SEQ ID NO:26.

11. The recombinant yeast cell of claim 10, wherein the first exogenous nucleic acid sequence comprises a sequence having at least 90% identity to SEQ ID NO:26.

12. The recombinant yeast cell of claim 11, wherein the first exogenous nucleic acid sequence comprises a sequence having at least 95% identity to SEQ ID NO:26.

13. The recombinant yeast cell of claim 12, wherein the first exogenous nucleic acid sequence comprises SEQ ID NO:26.

14. The recombinant yeast cell of claim 13, wherein the first exogenous nucleic acid sequence comprises two copies of SEQ ID NO:26.

15. The recombinant yeast cell of claim 13, wherein the first exogenous nucleic acid sequence comprises three copies of SEQ ID NO:26.

16. The recombinant yeast cell of any of claims 1-8, wherein the first exogenous nucleic acid sequence comprises a sequence having at least 75% identity to SEQ ID NO:27.

17. The recombinant yeast cell of claim 16, the first exogenous nucleic acid sequence comprises a sequence having at least 80% identity to SEQ ID NO:27.

18. The recombinant yeast cell of claim 17, wherein the first exogenous nucleic acid sequence comprises a sequence having at least 90% identity to SEQ ID NO:27.

19. The recombinant yeast cell of claim 18, wherein the first exogenous nucleic acid sequence comprises a sequence having at least 95% identity to SEQ ID NO:27.

20. The recombinant yeast cell of claim 19, wherein the first exogenous nucleic acid sequence comprises SEQ ID NO:27.

21. The recombinant yeast cell of claim 20, wherein the first exogenous nucleic acid sequence comprises two copies of SEQ ID NO:27.

22. The recombinant yeast cell of claim 20, wherein the first exogenous nucleic acid sequence comprises three copies of SEQ ID NO:27.

23. The recombinant yeast cell of any of claims 1-22, wherein the phosphotransacetylase protein comprises a sequence having at least 90% identity to SEQ ID NO:29.

24. The recombinant yeast cell of claim 23, wherein the phosphotransacetylase protein comprises a sequence having at least 95% identity to SEQ ID NO:29

25. The recombinant yeast cell of claim 24, wherein the phosphotransacetylase protein comprises a sequence having at least 98% identity to SEQ ID NO:29

26. The recombinant yeast cell of claim 25, wherein the phosphotransacetylase protein comprises SEQ ID NO:29

27. The recombinant yeast cell of any of claims 23-26, wherein the second exogenous nucleic acid sequence comprises a sequence having at least 75% identity to SEQ ID NO:23.

28. The recombinant yeast cell of claim 27, wherein the second exogenous nucleic acid sequence comprises a sequence having at least 80% identity to SEQ ID NO:23.

29. The recombinant yeast cell of claim 28, wherein the second exogenous nucleic acid sequence comprises a sequence having at least 90% identity to SEQ ID NO:23.

30. The recombinant yeast cell of claim 29, wherein the second exogenous nucleic acid sequence comprises a sequence having at least 95% identity to SEQ ID NO:23.

31. The recombinant yeast cell of claim 30, wherein the second exogenous nucleic acid sequence comprises SEQ ID NO:23.

32. The recombinant yeast cell of claim 31, wherein the second exogenous nucleic acid sequence comprises two copies of SEQ ID NO:23.

33. The recombinant yeast cell of any of claims 1-22, wherein the phosphotransacetylase protein comprises a sequence having at least 90% to SEQ ID NO:30.

34. The recombinant yeast cell of claim 33, wherein the phosphotransacetylase protein comprises a sequence having at least 95% identity to SEQ ID NO:30.

35. The recombinant yeast cell of claim 34, wherein the phosphotransacetylase protein comprises a sequence having at least 98% identity to SEQ ID NO:30.

36. The recombinant yeast cell of claim 36, wherein the phosphotransacetylase protein comprises SEQ ID NO:30.

37. The recombinant yeast cell of any of claims 33-36, wherein the second exogenous nucleic acid sequence comprises a sequence having at least 75% identity to SEQ ID NO:24.

38. The recombinant yeast cell of claim 37, wherein the second exogenous nucleic acid sequence comprises a sequence having at least 80% identity to SEQ ID NO:24.

39. The recombinant yeast cell of claim 38, wherein the second exogenous nucleic acid sequence comprises a sequence having at least 90% identity to SEQ ID NO:24.

40. The recombinant yeast cell of claim 39, wherein the second exogenous nucleic acid sequence comprises a sequence having at least 95% identity to SEQ ID NO:24.

41. The recombinant yeast cell of claim 40, wherein the second exogenous nucleic acid sequence comprises SEQ ID NO:24.

42. The recombinant yeast cell of claim 41, wherein the second exogenous nucleic acid sequence comprises two copies of SEQ ID NO:24.

43. The recombinant yeast cell of any of claims 33-36, wherein the second exogenous nucleic acid sequence comprises a sequence having at least 75% identity to SEQ ID NO:25.

44. The recombinant yeast cell of claim 43, wherein the second exogenous nucleic acid sequence comprises a sequence having at least 80% identity to SEQ ID NO:25.

45. The recombinant yeast cell of claim 44, wherein the second exogenous nucleic acid sequence comprises a sequence having at least 90% identity to SEQ ID NO:25.

46. The recombinant yeast cell of claim 45, wherein the second exogenous nucleic acid sequence comprises a sequence having at least 95% identity to SEQ ID NO:25.

47. The recombinant yeast cell of claim 46, wherein the second exogenous nucleic acid sequence comprises SEQ ID NO:25.

48. The recombinant yeast cell of claim 47, wherein the second exogenous nucleic acid sequence comprises two copies of SEQ ID NO:25.

49. The recombinant yeast cell of any of claims, wherein the recombinant yeast cell has reduced expression of an endogenous functional phosphofructokinase protein compared to a wild type yeast cell.

50. The recombinant yeast cell of any of claims 1-49, wherein the recombinant yeast cell does not express an endogenous functional phosphofructokinase protein.

51. The recombinant yeast cell of any of claims 1-50, wherein the recombinant yeast cell is an Arxula cell, a Saccharomyces cell, or a Yarrowia cell.

52. The recombinant yeast cell of claim 51, wherein the yeast cell is a Arxula cell.

53. The recombinant yeast cell of claim 52, wherein the recombinant yeast cell is Arxula adeninivorans

54. The recombinant yeast cell of claim 52 or 53, wherein the first exogenous nucleic acid sequence does not comprise one or more of TTA, ATA, and AGG.

55. The recombinant yeast cell of claim 54, wherein the first exogenous nucleic acid sequence does not comprise any of TTA, ATA, and AGG.

56. The recombinant yeast cell of claim 51, wherein the yeast cell is a Saccharomyces cell.

57. The recombinant yeast cell of claim 56, wherein the recombinant yeast cell is Saccharomyces cerevisiae.

58. The recombinant yeast cell of claim 56 or 57, wherein the first exogenous nucleic acid sequence does not comprise one or more of CGC, CGA, and TCG.

59. The recombinant yeast cell of claim 58, wherein the first exogenous nucleic acid sequence does not comprise any of CGC, CGA, and TCG.

60. The recombinant yeast cell of claim 51, wherein the yeast cell is a Yarrowia cell,

61. The recombinant yeast cell of claim 60, wherein the recombinant yeast cell is Yarrowia lipolytica.

62. The recombinant yeast cell of claim 60 or 61, wherein the first exogenous nucleic acid sequence does not comprise one or more of ATA, CTA, and AGT.

63. The recombinant yeast cell of claim 61 wherein the first exogenous nucleic acid sequence does not comprise any of ATA, CTA, and AGT.

64. The recombinant yeast cell of any of claims 1-63, wherein the recombinant yeast cell is Arxula adeninivorans, Saccharomyces cerevisiae, or Yarrowia lipolytica.

65. The recombinant yeast cell of any of claims 1-64, wherein the recombinant yeast cell is Yarrowia lipolytica.

66. The recombinant yeast cell of any of claims 1-65, wherein the first exogenous nucleic acid sequence and the second exogenous nucleic acid sequence are on a single expression cassette.

67. The recombinant yeast cell of any of claims 1-65, wherein the first exogenous nucleic acid sequence and the second exogenous nucleic acid sequence are controlled by a single promoter.

68. The recombinant yeast cell of any of claims 1-65, wherein the first exogenous nucleic acid sequence and the second exogenous nucleic acid sequence are on different expression cassettes.

69. The recombinant yeast cell of any of claims 1-65, wherein the first exogenous nucleic acid sequence and the second exogenous nucleic acid sequence are controlled by different promoters.

70. A method for generating the recombinant yeast cell of any of claims 1-69, the method comprising transforming a yeast cell with the first exogenous nucleic acid sequence and the second exogenous nucleic acid sequence.

71. A method for expressing a phosphoketolase protein and a phosphotransacetylase protein, the method comprising culturing the recombinant yeast cell of any of claims 1-69 under conditions sufficient to express the first exogenous nucleic acid sequence and the second exogenous nucleic acid sequence.

72. The method of claim 71, wherein the method comprises culturing the recombinant yeast cell for at least 10 passages.

73. A method for collecting an acetyl-CoA derived product from a yeast cell, the method comprising:
(a) culturing a recombinant yeast cell comprising:
(i) a first exogenous nucleic acid sequence encoding a phosphoketolase protein comprising a sequence having at least 90% sequence identity to SEQ ID NO:28; and
(ii) a second exogenous nucleic acid sequence encoding a phosphotransacetylase protein
under conditions sufficient to express the first exogenous nucleic acid sequence and the second exogenous nucleic acid sequence; and
(b) collecting the acetyl-CoA derived product from the yeast cell.

74. The method of claim 73, wherein the phosphoketolase protein comprises a sequence having at least 95% identity to SEQ ID NO:28.

75. The method of claim 74, wherein the phosphoketolase protein comprises a sequence having at least 98% identity to SEQ ID NO:28.

76. The method of claim 75, wherein the phosphoketolase protein comprises a sequence having at least 99% identity to SEQ ID NO:28.

77. The method of claim 76, wherein the phosphoketolase protein comprises SEQ ID NO:28.

78. The method of any of claims 73-77, wherein the first exogenous nucleic acid sequence comprises a sequence having at least 75% identity to SEQ ID NO:26.

79. The method of claim 78, wherein the first exogenous nucleic acid sequence comprises a sequence having at least 80% identity to SEQ ID NO:26.

80. The method of claim 79, wherein the first exogenous nucleic acid sequence comprises a sequence having at least 90% identity to SEQ ID NO:26.

81. The method of claim 80, wherein the first exogenous nucleic acid sequence comprises a sequence having at least 95% identity to SEQ ID NO:26.

82. The method of claim 81, wherein the first exogenous nucleic acid sequence comprises SEQ ID NO:26.

83. The method of claim 82, wherein the first exogenous nucleic acid sequence comprises two copies of SEQ ID NO:26.

84. The method of claim 82, wherein the first exogenous nucleic acid sequence comprises three copies of SEQ ID NO:26.

85. The method of any of claims 73-77, wherein the first exogenous nucleic acid sequence comprises a sequence having at least 75% identity to SEQ ID NO:27.

86. The method of claim 85, wherein the first exogenous nucleic acid sequence comprises a sequence having at least 80% identity to SEQ ID NO:27.

87. The method of claim 86, wherein the first exogenous nucleic acid sequence comprises a sequence having at least 90% identity to SEQ ID NO:27.

88. The method of claim 87, wherein the first exogenous nucleic acid sequence comprises a sequence having at least 95% identity to SEQ ID NO:27.

89. The method of claim 75, wherein the first exogenous nucleic acid sequence comprises SEQ ID NO:27.

90. The method of claim 89, wherein the first exogenous nucleic acid sequence comprises two copies of SEQ ID NO:27.

91. The method of claim 89, wherein the first exogenous nucleic acid sequence comprises three copies of SEQ ID NO:27.

92. The method of claim 73-91, wherein the phosphotransacetylase protein comprises a sequence having at least 90% identity to SEQ ID NO:29.

93. The method of claim 92, wherein the phosphotransacetylase protein comprises a sequence having at least 95% identity to SEQ ID NO:29

94. The method of claim 93, wherein the phosphotransacetylase protein comprises a sequence having at least 98% identity to SEQ ID NO:29

95. The method of claim 94, wherein the phosphotransacetylase protein comprises SEQ ID NO:29

96. The method of any of claims 92-95, wherein the second exogenous nucleic acid sequence comprises SEQ ID NO:23.

97. The method of claim 96, wherein the second exogenous nucleic acid sequence comprises two copies of SEQ ID NO:23.

98. The method of any of claims 73-91, wherein the phosphotransacetylase protein comprises a sequence having at least 90% to SEQ ID NO:30.

99. The method of claim 98, wherein the phosphotransacetylase protein comprises a sequence having at least 95% identity to SEQ ID NO:30.

100. The method of claim 99, wherein the phosphotransacetylase protein comprises a sequence having at least 98% identity to SEQ ID NO:30.

101. The method of claim 100, wherein the phosphotransacetylase protein comprises SEQ ID NO:30.

102. The method of any of claims 98-101, wherein the second exogenous nucleic acid sequence comprises a sequence having at least 75% identity to SEQ ID NO:24.

103. The method of claim 102, wherein the second exogenous nucleic acid sequence comprises a sequence having at least 80% identity to SEQ ID NO:24.

104. The method of claim 103, wherein the second exogenous nucleic acid sequence comprises a sequence having at least 90% identity to SEQ ID NO:24.

105. The method of claim 104, wherein the second exogenous nucleic acid sequence comprises a sequence having at least 95% identity to SEQ ID NO:24.

106. The method of claim 105, wherein the second exogenous nucleic acid sequence comprises SEQ ID NO:24.

107. The method of claim 106, wherein the second exogenous nucleic acid sequence comprises two copies of SEQ ID NO:24.

108. The method of any of claims 98-101, wherein the second exogenous nucleic acid sequence comprises a sequence having at least 75% identity to SEQ ID NO:25.

109. The method of claim 108, wherein the second exogenous nucleic acid sequence comprises a sequence having at least 80% identity to SEQ ID NO:25.

110. The method of claim 109, wherein the second exogenous nucleic acid sequence comprises a sequence having at least 90% identity to SEQ ID NO:25.

111. The method of claim 110, wherein the second exogenous nucleic acid sequence comprises a sequence having at least 95% identity to SEQ ID NO:25.

112. The method of claim 111, wherein the second exogenous nucleic acid sequence comprises SEQ ID NO:25.

113. The method of claim 112, wherein the second exogenous nucleic acid sequence comprises two copies of SEQ ID NO:25.

114. The method of any of claims 73-113, wherein the recombinant yeast cell has reduced expression of an endogenous functional phosphofructokinase protein compared to a wild type yeast cell.

115. The method of any of claims 73-114, wherein the recombinant yeast cell does not express a functional endogenous phosphofructokinase protein.

116. The method of any of claims 73-115, further comprising, prior to (a), transforming a yeast cell with the first exogenous nucleic acid sequence and the second exogenous nucleic acid sequence.

117. The method of any of claims 73-116, further comprising, prior to (c), eliminating expression of a functional endogenous phosphofructokinase protein in the yeast cell.

118. The method of any of claims 73-117, wherein the recombinant yeast cell is an Arxula cell, a Saccharomyces cell, or a Yarrowia cell.

119. The method of claim 118, wherein the yeast cell is a Arxula cell.

120. The method of claim 119, wherein the recombinant yeast cell is Arxula adeninivorans

121. The method of claim 119 or 120, wherein the first exogenous nucleic acid sequence does not comprise one or more of TTA, ATA, and AGG.

122. The method of claim 121, wherein the first exogenous nucleic acid sequence does not comprise any of TTA, ATA, and AGG.

123. The method of claim 118, wherein the yeast cell is a Saccharomyces cell.

124. The method of claim 123, wherein the recombinant yeast cell is Saccharomyces cerevisiae.

125. The method of claim 123 or 124, wherein the first exogenous nucleic acid sequence does not comprise one or more of CGC, CGA, and TCG.

126. The method of claim 125, wherein the first exogenous nucleic acid sequence does not comprise any of CGC, CGA, and TCG.

127. The method of claim 118, wherein the yeast cell is a Yarrowia cell,

128. The method of claim 127, wherein the recombinant yeast cell is Yarrowia lipolytica.

129. The method of claim 127 or 128, wherein the first exogenous nucleic acid sequence does not comprise one or more of ATA, CTA, and AGT.

130. The method of claim 129 wherein the first exogenous nucleic acid sequence does not comprise any of ATA, CTA, and AGT.

131. The method of any of claims 73-130, wherein, prior to (b), the yeast cell is cultured for at least 10 passages.

132. The method of any of claims 73-131, wherein the acetyl-CoA derived product is an oil.

133. The method of any of claims 73-131, wherein the acetyl-CoA derived product is a lipid.

134. The method of any of claims 73-131, wherein the acetyl-CoA derived product is a fatty acid.

135. The method of any of claims 73-131, wherein the acetyl-CoA derived product is a fatty alcohol.

136. The method of any of claims 73-131, wherein the acetyl-CoA derived product is a triacylglyceride.

137. The method of any of claims 73-131, wherein the acetyl-CoA derived product is triolein.

138. The method of any of claims 73-131, wherein the acetyl-CoA derived product is a terpene.

139. The method of any of claims 73-138, wherein the first exogenous nucleic acid sequence and the second exogenous nucleic acid sequence are on a single expression cassette.

140. The method of any of claims 73-138, wherein the first exogenous nucleic acid sequence and the second exogenous nucleic acid sequence are controlled by a single promoter.

141. The method of any of claims 73-138, wherein the first exogenous nucleic acid sequence and the second exogenous nucleic acid sequence are on different expression cassettes.

142. The method of any of claims 73-138, wherein the first exogenous nucleic acid sequence and the second exogenous nucleic acid sequence are controlled by different promoters.

143. A recombinant Yarrowia lipolytica cell, comprising:
(a) a first exogenous nucleic acid sequence encoding a phosphoketolase protein from Clostridium acetobutylicum, wherein the first exogenous nucleic acid sequence comprises a sequence having at least 95% identity to SEQ ID NO:27; and
(b) a second exogenous nucleic acid sequence encoding (i) a phosphotransacetylase protein from Bacillus subtilis or (ii) a phosphotransacetylase protein from Thermoanaerobacterium saccharolyticum.

144. The recombinant Yarrowia lipolytica cell of claim 143, wherein the first exogenous nucleic acid sequence comprises a sequence having at least 97% identity to SEQ ID NO:27.

145. The recombinant Yarrowia lipolytica cell of claim 144, wherein the first exogenous nucleic acid sequence comprises a sequence having at least 98% identity to SEQ ID NO:27.

146. The recombinant Yarrowia lipolytica cell of claim 145, wherein the first exogenous nucleic acid sequence comprises a sequence having at least 99% identity to SEQ ID NO:27.

147. The recombinant Yarrowia lipolytica cell of claim 146, wherein the first exogenous nucleic acid sequence comprises SEQ ID NO:27.

148. The recombinant Yarrowia lipolytica cell of any of claims 143-147, wherein the second exogenous nucleic acid sequence comprises a sequence having at least 95% identity to SEQ ID NO:23.

149. The recombinant Yarrowia lipolytica cell of any of claim 148, wherein the second exogenous nucleic acid sequence comprises a sequence having at least 98% identity to SEQ ID NO:23.

150. The recombinant Yarrowia lipolytica cell of any of claim 149, wherein the second exogenous nucleic acid sequence comprises a sequence having at least 99% identity to SEQ ID NO:23.

151. The recombinant Yarrowia lipolytica cell of any of claim 150, wherein the second exogenous nucleic acid sequence comprises SEQ ID NO:23.

152. The recombinant Yarrowia lipolytica cell of any of claims 143-147, wherein the second exogenous nucleic acid sequence comprises a sequence having at least 95% identity to SEQ ID NO:24.

153. The recombinant Yarrowia lipolytica cell of any of claim 152, wherein the second exogenous nucleic acid sequence comprises a sequence having at least 95% identity to SEQ ID NO:24.

154. The recombinant Yarrowia lipolytica cell of any of claim 153, wherein the second exogenous nucleic acid sequence comprises a sequence having at least 98% identity to SEQ ID NO:24.

155. The recombinant Yarrowia lipolytica cell of any of claim 154, wherein the second exogenous nucleic acid sequence comprises a sequence having at least 99% identity to SEQ ID NO:24.

156. The recombinant Yarrowia lipolytica cell of any of claim 155, wherein the second exogenous nucleic acid sequence comprises SEQ ID NO:24.

157. The recombinant Yarrowia lipolytica cell of any of claims 143-147, wherein the second exogenous nucleic acid sequence comprises a sequence having at least 95% identity to SEQ ID NO:25.

158. The recombinant Yarrowia lipolytica cell of any of claim 157, wherein the second exogenous nucleic acid sequence comprises a sequence having at least 98% identity to SEQ ID NO:25.

159. The recombinant Yarrowia lipolytica cell of any of claim 158, wherein the second exogenous nucleic acid sequence comprises a sequence having at least 99% identity to SEQ ID NO:25.

160. The recombinant Yarrowia lipolytica cell of any of claim 159, wherein the second exogenous nucleic acid sequence comprises SEQ ID NO:25.

161. The recombinant Yarrowia lipolytica cell of any of claims 143-160, wherein the recombinant yeast cell does not express a functional endogenous phosphofructokinase protein.


SEQUENCE LISTING (27 sequences)

SEQ ID NO: 1 - DNA, 47 bp, Yarrowia lipolytica
cgcaaaacagacggacactgaaccccccgcgcttcaaaacaccgaca

SEQ ID NO: 2 - DNA, 570 bp, Saccharomyces cerevisiae
atgaccactctggatgacaccgcttaccgataccgaacttccgttcctggcgatgccgaggctattgaggctctggatggatctttcaccactgacaccgttttccgagtgaccgctactggcgacggcttcaccctgcgagaggtgcctgtcgaccctcctctcaccaaggttttccctgacgatgagtcggacgatgagtctgacgctggagaggacggcgaccctgactctcgaactttcgtggcttacggcgacgatggagacctggccggctttgtggtcgtttcttactccggatggaaccgacgactgaccgtggaggacatcgaggtcgctcctgagcaccgaggtcatggtgtcggacgagctctgatgggtctcgctactgagttcgctcgagagcgaggtgctggccacctgtggctcgaggtcaccaacgttaacgcccctgctattcatgcctaccgacgaatgggttttaccctgtgtggcctcgatactgccctgtacgacggaaccgcttccgatggagagcaggccctctacatgtcgatgccctgcccttaa

SEQ ID NO: 3 - DNA, 275 bp, Saccharomyces cerevisiae
acaggccccttttcctttgtcgatatcatgtaattagttatgtcacgcttacattcacgccctcctcccacatccgctctaaccgaaaaggaaggagttagacaacctgaagtctaggtccctatttattttttttaatagttatgttagtattaagaacgttatttatatttcaaatttttcttttttttctgtacaaacgcgtgtacgcatgtaacattatactgaaaaccttgcttgagaaggttttgggacgctcgaaggctttaatttgc

SEQ ID NO: 4 - DNA, 406 bp, Yarrowia lipolytica
agagaccgggttggcggcgcatttgtgtcccaaaaaacagccccaattgccccaattgaccccaaattgacccagtagcgggcccaaccccggcgagagcccccttctccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaatcgttaagcatttccttctgagtataagaatcattcaaa

SEQ ID NO: 5 - DNA, 1131 bp, Artificial Sequence
  Synthetic Polynucleotide
atggcttcttacccttgccaccagcacgcttccgcttttgaccaggccgcccgatcccgaggacactccaaccgacgaaccgctctgcgaccccgacgacagcaggaggctaccgaggttcgactggagcagaagatgcctactctgctccgagtgtacatcgacggaccccacggtatgggcaagaccactaccactcagctgctcgtcgccctgggttcgcgagatgacattgtttacgtgcctgagcccatgacctactggcaggttctcggagcttctgagactatcgccaacatctacaccactcagcatcgactggaccagggagagatctccgctggagatgccgctgtggtcatgacctcggcccagattactatgggcatgccttacgctgtcaccgacgctgttctggctcctcacatcggtggagaggctggatcttcccatgctcctcctcctgctctgaccctcatcttcgatcgacaccctattgccgctctgctctgttaccccgccgctcgatacctgatgggctctatgacccctcaggccgtgctggcttttgtcgccctcatccctcccaccctgcctggtactaacattgtgctgggtgctctccctgaggaccgacatatcgatcgactcgctaagcgacagcgacctggagagcgactggacctcgctatgctggccgctattcgacgagtgtacggcctgctcgctaacaccgtccgatacctccagggtggtggatcgtggcgagaggactggggacagctgtctggtaccgctgtgcctcctcagggtgctgagcctcagtccaacgctggacctcgaccccacatcggtgacaccctgttcactctctttcgagctcctgagctgctcgctcctaacggcgacctgtacaacgtcttcgcctgggctctggatgttctcgccaagcgactccgacctatgcacgtctttattctggactacgatcagtcgcccgctggatgtcgagatgccctgctccagctcacctctggcatggttcagactcatgtgaccactcctggatccatccccaccatttgcgatctggctcgaactttcgcccgagagatgggagaggccaactaa

SEQ ID NO: 6 - DNA, 400 bp, Yarrowia lipolytica
gctgcttgtacctagtgcaaccccagtttgttaaaaattagtagtcaaaaacttctgagttagaaatttgtgagtgtagtgagattgtagagtatcatgtgtgtccgtaagtgaagtgttattgactcttagttagtttatctagtactcgtttagttgacactgatctagtattttacgaggcgtatgactttagccaagtgttgtacttagtcttctctccaaacatgagagggctctgtcactcagtcggcctatgggtgagatggcttggtgagatctttcgatagtctcgtcaagatggtaggatgatgggggaatacattactgctctcgtcaaggaaaccacaatcagatcacaccatcctccatggtatccgatgactctcttctccacagt

SEQ ID NO: 7 - DNA, 450 bp, Yarrowia lipolytica
cttttgatccgatggttactttttatgttctattttacattagcgtggaaatagaccatgccatctttggcaccccggaaaaacttgatccaatagagttgttgggtggagctagtgactggcggcaattggagagcttctagaagacgaaccaggagcccgataggaactccgttggcgtgagtcggccccccagtagcaattcgaatcacgtgacgtggagttttccctcgcccgcgttcctggattgtcccggtgtgacgaggccgactggatttgatcacccaaccccacacgacgcataatgtaaatgtatcatcatacagtacatgcccgagtctaatgattggctggttcacggggaccgggagcgtccagtgggccgtatggcggtggttcaaacacctcagatcagctcactatcggctgagacaatccttaactgttggt

SEQ ID NO: 8 - DNA, 621 bp, Yarrowia lipolytica
ggggtttggtgttggaaattaggatatctatttgattaatgtagcttggttttggacaagaatgctgattgatacatccggtatcacttgtatacaacgtagggggcggtgtatgggtgaaaatttcgaaagactgagaatcgaactgggaggtttttaaactcctgaaactgtagttggtgaacgagccgaggagtattttagacccgaaacatttggtgcaaccagatacgccaggtaaacaatctacactacactacatgggacgagggttgaaaaggtggtgacagggatattgcagatgggattctcgtgatgatgagccgtcagcgtctcggcttgtctttgtgttcaacggcttgtctacgtctgctcgtatccacctgtcatgttggttgttccatgcttataggcctgtctgttgggggcagtaaggttttgagaagtgtgtagtatggggatgtattatcttggggttttggttgagatattagtaatctgagagtttgagggtatttctggcggtttgtgtatataagtccggctttttccaagtcgatatctccttttgttgttgttttggggccgcttttatttcttccagggctgtaggcgaaattc

SEQ ID NO: 9 - DNA, 68 bp, Artificial Sequence
  Synthetic Polynucleotide
cgcaaaacagacggacactgaaccccccgcgcttcaaaacaccgacaatgaccactctggatgacacc

SEQ ID NO: 10 - DNA, 23 bp, Artificial Sequence
  Synthetic Polynucleotide
tctccatcggaagcggttccgtc

SEQ ID NO: 11 - DNA, 23 bp, Artificial Sequence
  Synthetic Polynucleotide
aacttccgttcctggcgatgccg

SEQ ID NO: 12 - DNA, 21 bp, Artificial Sequence
  Synthetic Polynucleotide
gaatttcgcctacagccctgg

SEQ ID NO: 13 - DNA, 822 bp, Saccharomyces cerevisiae
gatccaactggcaccgctggcttgaacaacaataccagccttccaacttctgtaaataacggcggtacgccagtgccaccagtaccgttacctttcggtatacctcctttccccatgtttccaatgcccttcatgcctccaacggctactatcacaaatcctcatcaagctgacgcaagccctaagaaatgaataacaatactgacagtactaaataattgcctacttggcttcacatacgttgcatacgtcgatatagataataatgataatgacagcaggattatcgtaatacgtaatagttgaaaatctcaaaaatgtgtgggtcattacgtaaataatgataggaatgggattcttctatttttcctttttccattctagcagccgtcgggaaaacgtggcatcctctctttcgggctcaattggagtcacgctgccgtgagcatcctctctttccatatctaacaactgagcacgtaaccaatggaaaagcatgagcttagcgttgctccaaaaaagtattggatggttaataccatttgtctgttctcttctgactttgactcctcaaaaaaaaaaaatctacaatcaacagatcgcttcaattacgccctcacaaaaacttttttccttcttcttcgcccacgttaaattttatccctcatgttgtctaacggatttctgcacttgatttattataaaaagacaaagacataatacttctctatcaatttcagttattgttcttccttgcgttattcttctgttcttctttttcttttgtcatatataaccataaccaagtaatacatattcaaa

SEQ ID NO: 14 - DNA, 1026 bp, Artificial Sequence
  Synthetic Polynucleotide
atgaagaagcccgagctgaccgctacctctgttgagaagttcctgattgagaagtttgattccgtttccgacctgatgcagctgtccgagggcgaggagtctcgagccttctcctttgacgtgggcggacgaggttacgttctgcgagtgaactcgtgtgccgacggcttctacaaggatcgatacgtctaccgacactttgcttctgccgctctgcccatccctgaggttctcgacattggcgagttctctgagtccctcacctactgcatctctcgacgagctcagggagtcaccctgcaggacctccctgagactgagctgcctgctgtcctccagcctgttgctgaggccatggacgctatcgctgctgctgatctgtcccagacctcgggtttcggcccctttggacctcagggaattggacagtacaccacttggcgagacttcatctgtgctattgccgatcctcacgtctaccattggcagaccgttatggacgatactgtgtcggcttctgtcgctcaggctctggacgagctgatgctctgggccgaggattgccccgaggttcgacacctggtgcatgctgacttcggttccaacaacgttctcaccgacaacggccgaatcactgccgtgattgactggtccgaggctatgtttggcgactcgcagtacgaggtggccaacatcttcttttggcgaccctggctggcttgtatggagcagcagacccgatacttcgagcgacgacatcctgagctcgctggatcccctcgactgcgagcttacatgctccgaattggtctggaccagctctaccagtcgctggtggatggcaactttgacgatgctgcctgggctcagggacgatgtgacgccatcgtgcgatctggcgctggaaccgtcggacgaactcagattgcccgacgatccgctgctgtctggaccgacggatgcgtggaggtcctggctgattcgggtaaccgacgaccctctactcgacctcgagctaaggagtaa

SEQ ID NO: 15 - DNA, 205 bp, Saccharomyces cerevisiae
gttaattcaaattaattgatatagttttttaatgagtattgaatctgtttagaaataatggaatattatttttatttatttatttatattattggtcggctcttttcttctgaaggtcaatgacaaaatgatatgaaggaaataatgatttctaaaattttacaacgtaagatatttttacaaaagcctagctcatcttttgtca

SEQ ID NO: 16 - DNA, 999 bp, Yarrowia lipolytica
gagtttggcgcccgttttttcgagccccacacgtttcggtgagtatgagcggcggcagattcgagcgtttccggtttccgcggctggacgagagcccatgatgggggctcccaccaccagcaatcagggccctgattacacacccacctgtaatgtcatgctgttcatcgtggttaatgctgctgtgtgctgtgtgtgtgtgttgtttggcgctcattgttgcgttatgcagcgtacaccacaatattggaagcttattagcctttctattttttcgtttgcaaggcttaacaacattgctgtggagagggatggggatatggaggccgctggagggagtcggagaggcgttttggagcggcttggcctggcgcccagctcgcgaaacgcacctaggaccctttggcacgccgaaatgtgccacttttcagtctagtaacgccttacctacgtcattccatgcatgcatgtttgcgccttttttcccttgcccttgatcgccacacagtacagtgcactgtacagtggaggttttgggggggtcttagatgggagctaaaagcggcctagcggtacactagtgggattgtatggagtggcatggagcctaggtggagcctgacaggacgcacgaccggctagcccgtgacagacgatgggtggctcctgttgtccaccgcgtacaaatgtttgggccaaagtcttgtcagccttgcttgcgaacctaattcccaattttgtcacttcgcacccccattgatcgagccctaacccctgcccatcaggcaatccaattaagctcgcattgtctgccttgtttagtttggctcctgcccgtttcggcgtccacttgcacaaacacaaacaagcattatatataaggctcgtctctccctcccaaccacactcacttttttgcccgtcttcccttgctaacacaaaagtcaagaacacaaacaaccaccccaacccccttacacacaagacatatctacagca

SEQ ID NO: 17 - DNA, 300 bp, Yarrowia lipolytica
gcgtctacaactggacccttagcctgtatatatcaattgattatttaaagatttggtcggtaggcggttcgtattgtacaatgggatctgttactgaggtggatctacccaacttgcgagattcaattgcgagattcaatcgcgagattcaattgcgagaatcagttgcgagttgttctaacactcagcttctacgagcgcttgtattaggacgagtgatactccgtggggcgacggcttctcttgcgtcttctgttgtattctttcttacactatcgtccatctccaaccacctcgtac

SEQ ID NO: 18 - DNA, 862 bp, Yarrowia lipolytica
agctagctcgtcgtgttcaggaactgttcgatggttcggagagagtcgccgcccagaacatacgcgcaccgatgtcagcagacagccttattacaagtacagtatgtacatactactgtatattcaagcaagtatatccgtagggtgcgggtgatttggatctaaggttcgtactcaacactcacgagcagcttgcctatgttacatccttttatcagacataacataattggagtttacttacacacggggtgtacctgtatgagcaccacctacaattgtagcactggtacttgtacaaagaatttattcgtacgaatcacaggaacggccgccctcaccgaaccagcgaatacctcagcggtcccctgcagtgactcaacaaagcgatatgaacatcttgcgatggtatcctgctgatagtttttactgtacaaacacctgtgtagctccttctagcatttttaagttattcacacctcaaggggagggataaattaaataaattccaaaagcgaagatcgagaaactaaattaaaattccaaaaacgaagttggaacacaaccccccgaaaaaaaacaacaagcaaaaaacccaacaaaataaacaaaaacaaaataaatatataacaaaataaatatataactaccagtatctgactaaaagttcaaatactcgtacttacaacaaatagaaatgagccggccaaaattctgcagaaaaaattttcaaacaagtactttcaaacaagtactggtataattaaattaaaaaacacatcaaagtatcataacgttagttattttattttatttaataaaagaaaacaacaaaatgggctcaaaactttcaacttatacg

SEQ ID NO: 19 - DNA, 241 bp, Yarrowia lipolytica
atacataccaaataacaatttagtatttatctaagtgcttttcgtagataatggaatacaaatggatatccagagtatacacatggatagtatacactgacacgacaattctgtatctctttatgttaactactgtgaggcgttaaatagagcttgatatataaaatgttacatttcacagtctgaacttttgcagattacctaatttggtaagatattaattatgaactgaaagttgatg

SEQ ID NO: 20 - DNA, 877 bp, Arxula adeninivorans
tgcgtcggaacgggatatgcattcccctagtttcgccgcagtgcagaatcaggcggtttctttgcaccacaccacatacggaggatgacgggcattattgatgttgaatagtaacctgatcgtgactagtatgacggaacccaacagcaacagccgaccgtttgtgagcgtttttgcggccggtcaggcgagtttttccggcctgccaatggtccttccgtaccctttaccctgtacgctgtacctgccacggataggccgtgctccacctgctcactatggtgggtgcggggaaaacaacaggcaggctcaattgctctgcaaatgggttgagggggtgattgatgtcactggtacaccaacaggggaatgctcggcgttgattttgggccacctcttttgtttgccagagcttgtctctattgtcaaatttaacggtctgcaactgttgcccaaaatgggacaatgatccgatgcctgcatagacaccctgcttgagggtgcgatcgccctaatacgaggcaaaccaagttttccaattgaccttcaattgacgagcggttgttgcgacaggggactggagtgctacctgtttagagttcaaatccgtcacccagcattgaaagtttttccccgcattggatgattgcaatgccgctaacccgctcatccgccaaagttcatagtcccaccctgcctcgacttatcggaccacatggggctcccttatgcgcgcgcatatggcgcttgattgctttttggtcaacgtttgggacaaatttcctttgttaaggcggacccgccagcagatacgaaggtataaatagggctcactttcaccatcttgtccattcaattgcaagactcaaaagtaata

SEQ ID NO: 21 - DNA, 570 bp, Artificial Sequence; identical to SEQ ID NO: 2
  Synthetic Polynucleotide

SEQ ID NO: 22 - DNA, 301 bp, Artificial Sequence
  Synthetic Polynucleotide
gcggtttagattttccaattgtaaatatattactgtaccattctgtactaaataacgtgttttttatactacttcctatctatattctatatcgttactggcatatatatatcgttgctggaggtcgaaggtgaaatttcacttgcctttctcttccccgagccgcacgccgccatatcgttatcttgattgagcccgacatcatgatcactaactgattatgctccagcaggagcaggttgtagagctccaagatttgcgaggaggtggagagaatagcacgggacagagagtacgatgg

SEQ ID NO: 23 - DNA, 972 bp, Artificial Sequence
  Synthetic Polynucleotide
atggccgatttgttctctaccgttcaagaaaaagttgctggtaaggatgtcaagatcgtttttccagaaggtttggacgaaagaattttggaagctgtttctaaattggccggtaacaaggttttgaacccaatcgttattggtaacgaaaacgaaattcaagccaaggccaaagaattgaacttgactttgggtggtgttaagatctacgatccacatacttatgaaggtatggaagatttggttcaagccttcgttgaaagaagaaaaggtaaggctactgaagaacaagctagaaaagctttgttagacgaaaactacttcggtactatgttggtctacaaaggtttggctgatggtttggtttctggtgctgctcattctactgctgatactgttagaccagcattgcaaatcatcaagacaaaagaaggtgtcaaaaagacctccggtgttttcattatggctagaggtgaagaacaatacgttttcgctgattgcgctattaacattgctccagattctcaagatttggccgaaattgctattgaatctgctaacactgctaagatgttcgacattgaacctagagttgctatgttgtcattctctacaaaaggttctgctaagtctgacgaaactgaaaaggttgctgatgcagttaagatcgctaaagaaaaagctccagaattgaccttggatggtgaatttcaatttgatgctgctttcgttccatccgttgctgaaaaaaaagcaccagattctgaaatcaagggtgatgccaatgttttcgtattcccatctttagaagctggtaacatcggttacaagattgctcaaagattgggtaactttgaagctgttggtccaatattgcaaggtttgaatatgccagttaacgatttgtctagaggttgcaatgcagaagatgtttacaacttggctttgattactgctgctcaagctttgtaa

SEQ ID NO: 24 - DNA, 987 bp, Artificial Sequence
  Synthetic Polynucleotide
atgtccatcatccaaaacatcatcgaaaaggccaagtccgataagaagaaaatcgttttgccagaaggtgctgaacctagaactttgaaagctgctgaaatcgtcttgaaagaaggtattgctgatttggtcttgttgggtaacgaagacgaaattagaaatgctgccaaggatttggatatctccaaggccgaaattatcgatccagttaagtctgaaatgttcgacagatacgctaacgacttctacgaattgagaaagaacaagggtatcaccttggaaaaggctagagaaaccattaaggacaacatctacttcggttgcatgatggtcaaagaaggttacgctgacggtttggtttctggtgctattcatgctacagctgatttgttaagaccagccttccaaattatcaaaactgctccaggtgctaagatcgtcagttcatttttcattatggaagtcccaaactgcgaatacggtgaaaatggtgtttttttgttcgctgattgtgccgttaatccatctccaaatgctgaagaattggcttccattgctgttcaatctgctaatactgctaagaatttgttgggtttcgaacctaaggttgccatgttgtctttttcaacaaaaggttccgcttcccatgaattggttgataaggttagaaaggctaccgaaatcgccaaagaattgatgccagatgttgctattgatggtgaattacaattggatgctgccttggtaaaagaagttgctgaattgaaagctccaggttcaaaagttgctggttgtgctaatgttttgatcttcccagacttacaagctggtaacattggttacaagttggttcaaagattggctaaggctaatgccattggtccaattactcaaggtatgggtgctccagttaatgatttgtctagaggttgttcctacagagatatcgttgatgttattgctactaccgctgttcaagctcaataa

SEQ ID NO: 25 - DNA, 987 bp, Artificial Sequence
  Synthetic Polynucleotide
atgtccatcatccagaacatcatcgagaaggccaagtctgacaagaagaagatcgttctgcccgagggcgctgagccccgaactctgaaggccgccgagatcgtgctgaaggaaggcattgccgacctggtgctgctgggcaacgaggacgagatccgaaacgccgccaaggacctggacatctctaaggccgagatcatcgaccccgtgaagtctgagatgttcgaccgatacgccaacgacttctacgagctgcgaaagaacaagggcatcaccctggaaaaggcccgagagactatcaaggacaacatctacttcggctgcatgatggtcaaggaaggctacgccgacggcctggtgtctggcgccatccacgccaccgccgacctgctgcgacccgccttccagatcatcaagactgcccctggcgccaagatcgtgtcctcgttcttcatcatggaagtgcccaactgcgagtacggcgagaacggcgtgttcctgttcgccgactgcgctgtgaacccctcgcctaacgccgaggaactggcctctatcgccgtgcagtctgccaacaccgctaagaacctgctgggcttcgagcccaaggtggccatgctgtctttctcgaccaagggctctgcctctcacgagctggtggacaaggtgcgaaaggctaccgagatcgccaaggaactgatgcccgacgtggccatcgacggcgaactgcagctggacgccgctctggtgaaggaagtggccgagctgaaggctcccggctctaaggtggccggctgcgccaacgtgctgatcttccccgacctgcaggccggcaacatcggctacaagctggtgcagcgactggccaaggccaacgccatcggacccatcactcaaggcatgggcgctcccgtgaacgacctgtctcgaggctgctcttaccgagacatcgtggacgtgatcgccaccaccgctgtgcaggcccagtaa

SEQ ID NO: 26 - DNA, 2391 bp, Artificial Sequence
  Synthetic Polynucleotide
atgcagtccatcatcggtaaacacaaggatgaaggtaagattacccctgaatacttgaagaagattgacgcttattggcgagctgccaactttatttctgtcggtcagctttacttgttggacaaccctttgttgcgagaacctttgaaacctgaacacttgaagcgaaaggttgttggtcactggggtactattcctggtcagaacttcatctacgcccacttgaaccgagtcatcaaaaagtacgatttggacatgatctacgtttctggtcctggtcacggtggtcaggttatggtttctaactcttacttggacggtacttactccgaagtttaccctaacgtttcccgagatttgaacggtttgaagaagttgtgcaagcagttttcattccctggtggtatctcttcacacatggctcctgaaactcctggttctattaacgaaggtggtgaattgggttattccttggctcactcttttggtgctgttttcgataaccctgatttgattactgcttgcgttgttggtgatggtgaagctgaaactggtcctttggctacatcttggcaggctaacaaatttttgaaccctgttactgatggtgccgttttgcctattcttcacttgaacggttacaagatctccaaccctactgtcttgtctcgaattcctaaggacgaattggaaaagttcttcgaaggtaacggttggaagccttactttgttgaaggtgaagatcctgaaaccatgcacaagttgatggctgaaactttggatatcgtcaccgaagaaatcttgaacattcagaagaacgcccgagaaaacaacgattgctctcgacctaaatggcctatgatcgttttgcgaactcctaaaggttggactggtcctaaattcgttgatggtgttcctaacgaaggttcttttcgagcacaccaggttcctttggcagttgatcgataccacaccgaaaacttggaccagttggaagaatggttgaagtcttacaagcctgaagaacttttcgacgaaaactaccgattgatccctgaacttgaagaattgacccctaagggtaacaagcgaatggctgctaacttgcacgctaacggtggtttgttgcttcgagaattgcgaacccctgatttccgagattacgctgttgatgttcctacacctggttcaactgttaagcaggatatgatcgaattgggtaaatacgtccgagatgtcgtcaagttgaacgaagatacacgaaacttccgaatcttcggtcctgacgaaactatgtctaaccgattgtgggctgtctttgaaggtactaagcgacagtggttgtccgaaatcaaagaacctaacgacgaattcttgtccaacgatggtcgaatcgttgactctatgttgtctgaacacttgtgtgaaggttggcttgaaggttacttgttgactggtcgacacggtttttttgcctcttacgaagctttcttgcgaatcgtcgattccatgattacccagcacggtaaatggttgaaagtcacctctcagttgccttggcgaaaggatattgcttccttgaacttgattgccacctctaacgtttggcagcaggatcacaacggttatacccaccaggaccctggtttgttgggtcacatagttgataagaagcctgaaatcgttcgagcttatttgcctgctgatgctaacactttgttggccgtttttgataagtgcttgcacaccaagcacaagatcaacttgttggttacctctaaacaccctcgacagcagtggcttactatggatcaggccgttaagcacgttgaacagggtatttctatttgggattgggcttctaacgataagggtcaggaacctgatgttgttattgcttcctgtggtgatactcctactttggaagctttggctgctgttaccattcttcacgaacacttgcctgaattgaaggtccgattcgttaacgttgttgacatgatgaagttgttgcctgaaaacgaacaccctcacggtttgtctgataaggattacaacgctttgttcactaccgataagcctgttatttttgcctttcacggtttcgcccacttgatcaaccagttgacctaccaccgagaaaaccgaaacttgcacgttcacggttacatggaagaaggtacaattactactcctttcgacatgcgagtccagaacaagttggaccgattcaacttggttaaggacgtcgttgaaaacttgcctcagcttggtaaccgaggtgcccacttggttcagttgatgaacgataagttggtcgaacacaaccagtatatccgagaagttggtgaagatttgcctgaaattaccaactggcagtggcacgtttga

SEQ ID NO: 27 - DNA, 2391 bp, Artificial Sequence
  Synthetic Polynucleotide
atgcagtccatcatcggaaagcacaaggatgagggaaagattacccccgagtacctcaagaagattgacgcttactggcgagctgccaacttcatttctgtcggacagctctacctcctcgacaaccccctcctccgagagcccctcaagcccgagcacctcaagcgaaaggtggtgggacactggggaaccattcccggacagaacttcatctacgcccacctcaaccgagtcatcaagaagtacgatctcgacatgatctacgtgtctggacccggacacggaggacaggtgatggtgtctaactcttacctcgacggaacctactccgaggtgtaccccaacgtgtcccgagatctcaacggactcaagaagctctgcaagcagttctctttccccggaggaatctcttctcacatggctcccgagacccccggatctattaacgagggaggagagctcggatactccctcgctcactctttcggagctgtgttcgataaccccgatctcattaccgcttgcgtggtgggagatggagaggctgagaccggacccctcgctacctcttggcaggctaacaagttcctcaaccccgtgaccgatggagccgtgctccccattctccacctcaacggatacaagatctccaaccccaccgtcctctctcgaattcccaaggacgagctcgagaagttcttcgagggaaacggatggaagccctacttcgtggagggagaggatcccgagaccatgcacaagctcatggctgagaccctcgatatcgtcaccgaggagatcctcaacattcagaagaacgcccgagagaacaacgattgctctcgacccaagtggcccatgatcgtgctccgaacccccaagggatggaccggacccaagttcgtggatggagtgcccaacgagggatctttccgagctcaccaggtgcccctcgctgtggatcgataccacaccgagaacctcgaccagctcgaggagtggctcaagtcttacaagcccgaggagctcttcgacgagaactaccgactcatccccgagctcgaggagctcacccccaagggaaacaagcgaatggctgctaacctccacgctaacggaggactcctcctccgagagctccgaacccccgatttccgagattacgctgtggatgtgcccacccccggatctaccgtgaagcaggatatgatcgagctcggaaagtacgtccgagatgtcgtcaagctcaacgaggatacccgaaacttccgaatcttcggacccgacgagaccatgtctaaccgactctgggctgtcttcgagggaaccaagcgacagtggctctccgagatcaaggagcccaacgacgagttcctctccaacgatggacgaatcgtggactctatgctctctgagcacctctgtgagggatggctcgagggatacctcctcaccggacgacacggattcttcgcctcttacgaggctttcctccgaatcgtcgattccatgattacccagcacggaaagtggctcaaggtcacctctcagctcccctggcgaaaggatattgcttccctcaacctcattgccacctctaacgtgtggcagcaggatcacaacggatacacccaccaggaccccggactcctcggacacattgtggataagaagcccgagatcgtgcgagcttacctccccgctgatgctaacaccctcctcgccgtgttcgataagtgcctccacaccaagcacaagatcaacctcctcgtgacctctaagcacccccgacagcagtggctcaccatggatcaggccgtgaagcacgtggagcagggaatttctatttgggattgggcttctaacgataagggacaggagcccgatgtggtgattgcttcctgtggagatacccccaccctcgaggctctcgctgctgtgaccattctccacgagcacctccccgagctcaaggtccgattcgtgaacgtggtggacatgatgaagctcctccccgagaacgagcacccccacggactctctgataaggattacaacgctctcttcaccaccgataagcccgtgattttcgccttccacggattcgcccacctcatcaaccagctcacctaccaccgagagaaccgaaacctccacgtgcacggatacatggaggagggaaccattaccacccccttcgacatgcgagtccagaacaagctcgaccgattcaacctcgtgaaggacgtcgtggagaacctcccccagctcggaaaccgaggagcccacctcgtgcagctcatgaacgataagctcgtcgagcacaaccagtacatccgagaggtgggagaggatctccccgagattaccaactggcagtggcacgtgtga